(19) United States
(12) Patent Application Publication
(10) Pub. No.: US 2024/0335570 A1
(43) Pub. Date: Oct. 10, 2024

(54) FOLATE RECEPTOR-TARGETED CONJUGATES WITH BRUSH BORDER MEMBRANE ENZYME-CLEAVABLE LINKERS AND METHODS OF USE IN IMAGING AND TREATING CANCER

(71) Applicant: PURDUE RESEARCH FOUNDATION, West Lafayette, IN (US)

(72) Inventors: Philip LOW, West Lafayette, IN (US); Madduri SRINIVASARAO, West Lafayette, IN (US); Spencer LINDEMAN, West Lafayette, IN (US); Spencer GARDEEN, Waltham, MA (US)

(21) Appl. No.: 18/560,675

(22) PCT Filed: May 13, 2022
(86) PCT No.: PCT/US2022/029292

§ 371 Date: Nov. 13, 2023

Related U.S. Application Data
(60) Provisional application No. 63/188,910, filed on May 14, 2021.
(60) Provisional application No. 63/318,463, filed on Mar. 10, 2022.

(51) Int. Cl. A61K 51/04; A61B 6/00; A61K 49/00; A61K 51/08; A61P 35/00

(52) U.S. Cl. CPC A61K 51/0497; A61K 49/0032; A61K 51/088; A61P 35/00; A61B 6/481

(57) ABSTRACT

A conjugate of formula (I) FRTL-BBMecL-AA or (II) FRTL-Alb-BBMecL-AA, wherein FRTL is a folate receptor-targeting ligand, BBMecL is a brush border membrane (BBM) enzyme-cleavable linker, Alb is an albumin-binder moiety, and AA is an active agent; composition comprising same; and method of imaging and/or treating a tumor.

### PRIORITY

This patent application is related to and claims the priority benefit of U.S. Provisional Patent Application No. 63/188,910 filed May 14, 2021, and U.S. Provisional Patent Application No. 63/318,463 filed Mar. 10, 2022. The contents of both of the foregoing applications are hereby incorporated by reference in their entireties into this disclosure.

### TECHNICAL FIELD

The present disclosure relates to conjugates comprising a ligand that targets a receptor, an enzyme-cleavable linker, and an agent for imaging or therapeutically treating a tumor or tumor-associated macrophages.

### BACKGROUND

Folate receptor (FR) is a proven target for a variety of tumors and activated macrophages in humans. The FR binds to folic acid conjugates with high affinity and internalizes rapidly, which are ideal attributes for receptor-mediated drug delivery.

Folate ligand-targeted conjugates are used for imaging and treatment (e.g., imaging and treatment of cancer). A disadvantage of their use is high and long-term kidney retention. Kidney retention renders folate-based radiotherapeutics unusable due to the risk of damaging kidneys, which are radiosensitive. Fluorescent or radioactive imaging of renal carcinomas is also ineffective with folate-based conjugates.

This problem is currently being addressed in various ways. Antifolates are administered to block uptake to the kidney, the folate-ligand structure is modified, or plasma expanders or diuretics are administered to increase urination. Several strategies have already been employed to improve the critical tumor-to-kidney ratio for folate-targeted theranostics in preclinical studies. These include pretreatment antifolates (e.g., pemetrexed) and the incorporation of albumin-binders, but further optimizations are necessary to allow application of FR-targeted radiotherapy in human patients, in particular for the imaging and treatment of renal carcinomas.

In view of the above, the present disclosure seeks to provide a folate ligand-targeted conjugate that can be used without further structural modification or administration of other agents to address kidney retention. The conjugates can be used to image and treat tumors and tumor-associated macrophages, while providing improved pharmacokinetics. This and other objects and advantages, as well as additional inventive features, will be apparent from the detailed description provided herein.

### SUMMARY

Provided is a conjugate of formula I or formula II:

FRTL-BBMecL-AA (formula I)

or

FRTL-Alb-BBMecL-AA (formula II),

wherein:
FRTL is a folate receptor-targeting ligand,
BBMecL is a kidney brush border membrane (BBM) enzyme-cleavable linker,
Alb is an albumin-binder moiety, and
AA is an active agent.

The FRTL can have a molecular weight below 10,000. The Alb can associate noncovalently with serum albumin.

The FRTL can have the structure:

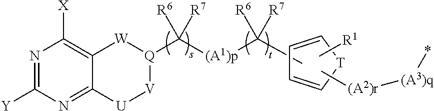

in which:
T is selected from the group consisting of S, O, NR^{4b}, and —HC═CH—,
U, V, and W represent divalent moieties, each independently selected from the group consisting of —(R^{6a})C═, —N═, —(R^{6a})C(R^{7a})—, and —N(R^{4a})—, wherein R^{6a} and R^{7a} are each independently selected from the group consisting of hydrogen, halo, and C₁-C₁₂ alkoxy; or R^{6a} and R^{7a} are taken together to form a carbonyl group,
X and Y are each independently selected from the group consisting of halo, R², OR², SR³, and NR⁴R⁵,
Q is C or CH,
A¹ and A² are each independently selected from the group consisting of oxygen, sulfur, —C(Z)—, —C(Z)O—, —OC(Z)—, —N(R^{4b})—, —C(Z)N(R^{4b})—, —N(R^{4b})C(Z)—, —OC(Z)N(R^{4b})—, —N(R^{4b})C(Z)O—, —N(R^{4b})C(Z)N(R^{5b})—, —S(O)—, —S(O)₂—, —N(R^{4b})S(O)₂—, —C(R^{6b})(R^{7b})—, —N(C≡CH)—, —N(CH₂C≡CH)—, C₁-C₁₂ alkylene, and C₁-C₁₂ alkyeneoxy, where Z is oxygen or sulfur,
R², R³, R⁴, R^{4a}, R^{4b}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl,
A³ is an amino acid,
R¹ is selected from the group consisting of hydrogen, halo, C₁-C₁₂ heteroalkyl, and C₁-C₁₂ alkoxy,
R⁶ and R⁷ are each independently selected from the group consisting of hydrogen, halo, C₁-C₁₂ alkyl, and C₁-C₁₂ alkoxy; or R⁶ and R⁷ are taken together to form a carbonyl group,
q is an integer from 1 to 3,
p, r, s, and t are each independently 0 or 1, and * indicates the point of attachment to the BBMecL or the Alb.
   Q can be CH. X can be OH and Y can be NH₂. W and U can be —N(R^{4a})—, Q can be CH, V can be CH₂, A¹ can be —N(R^{4b})—, s can be 1, p can be 1, and t can be 0. R^{4a} and R^{4b} can be independently alkyl or heteroalkyl. R^{4a} and R^{4b} can be methyl.

The FRTL can have the structure:

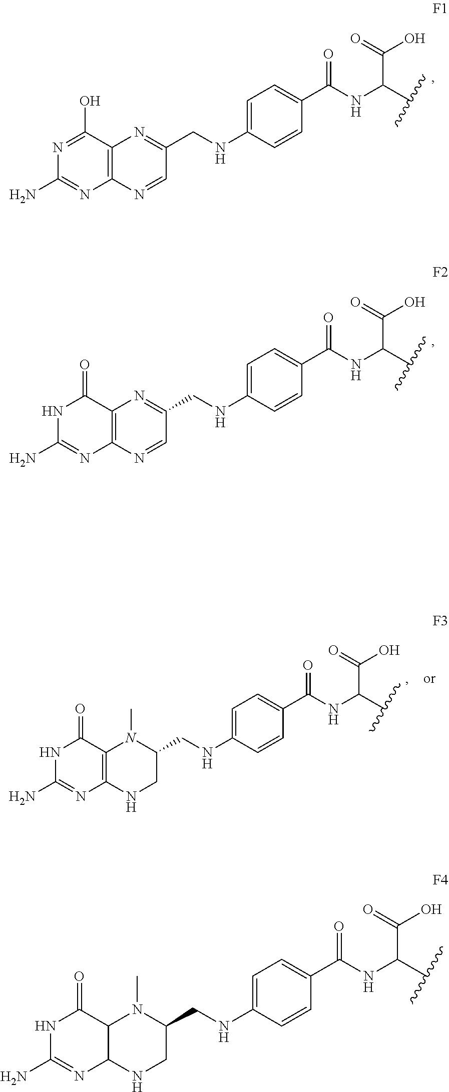

wherein

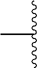

indicates a point of attachment of the FRTL to Alb or BBMecL.

Alb can have the structure:

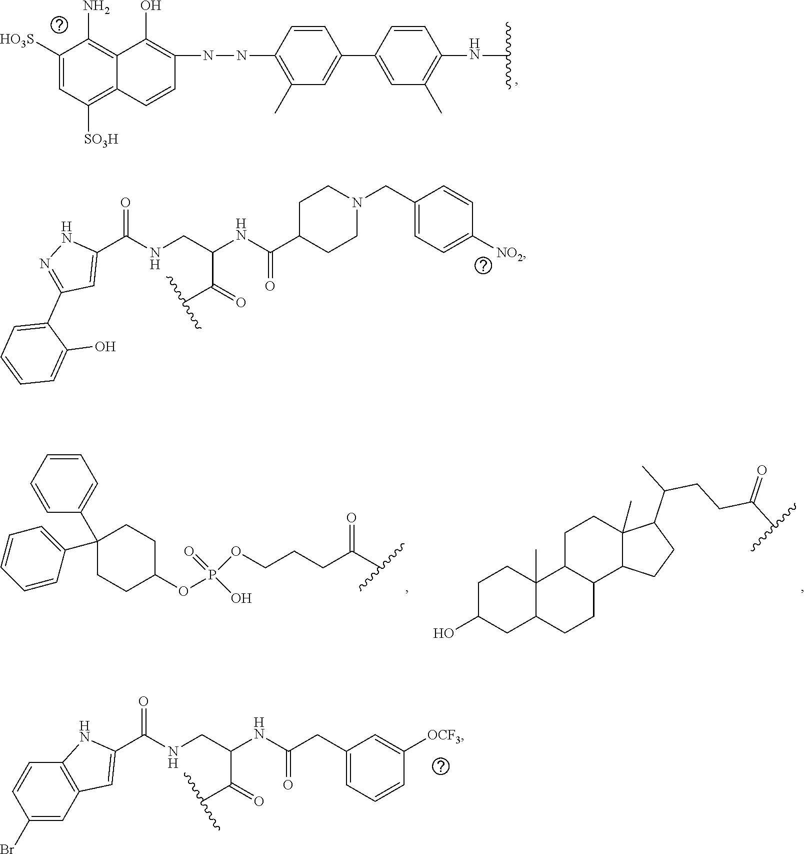 [SMILES: *Nc1ccc(-c2ccc(NNc3ccc4c(S(=O)(=O)O)cc(S(=O)(=O)O)c(N)c4c3O)c(C)c2)cc1C ; *C(=O)C(CNC(=O)c1cc(-c2ccccc2O)n[nH]1)NC(=O)C1CCN(Cc2ccc([N+](=O)[O-])cc2)CC1 ; *C(=O)CCCOP(=O)(O)OC1CCC(c2ccccc2)(c2ccccc2)CC1 ; *C(=O)CCC(C)C1CCC2C3CCC4CC(O)CCC4(C)C3CCC12C ; *C(=O)C(CNC(=O)c1cc2cc(Br)ccc2[nH]1)NC(=O)Cc1cccc(OC(F)(F)F)c1]

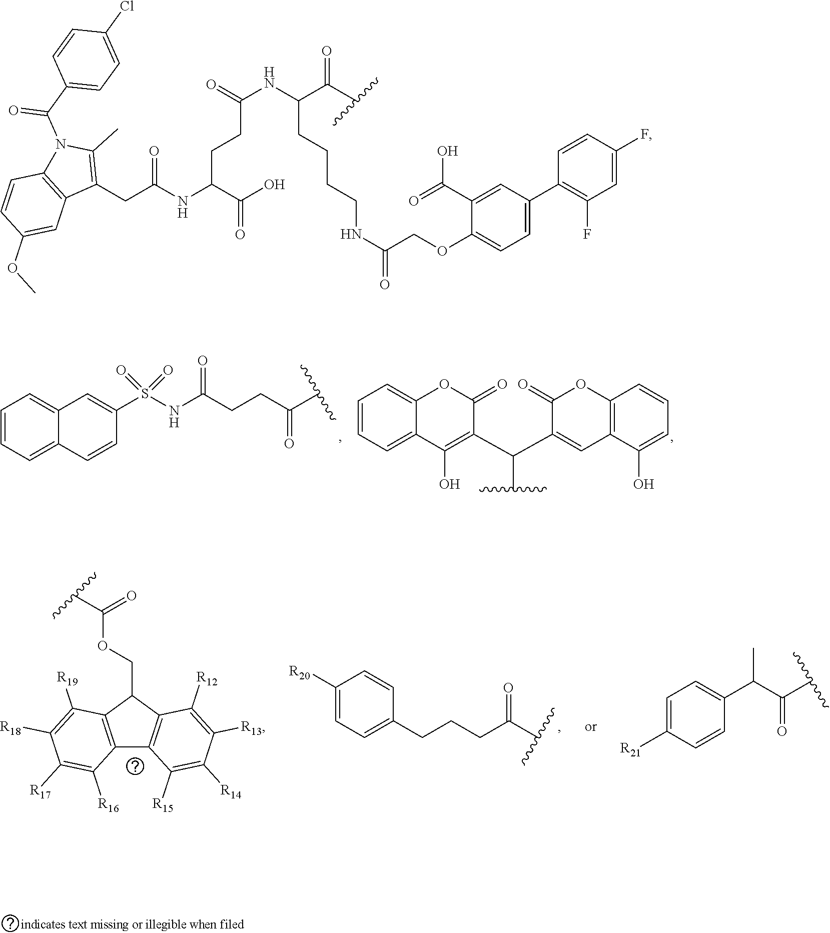

wherein R₁₂₋₁₉ are independently —H, —C₁-C₆ alkyl, —F, —Cl, —Br, —I, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, or —SO₂F; and
R₂₀ and R₂₁ are independently —H, —C₁-C₆ alkyl, —F, —Cl, —Br, —I, —O—C₁₋₆ alkyl, —CN, —CHO, —B(OH)₂, —C═C—C(O)aryl, —C═C—S(O)₂aryl, —CO₂H, —SO₃H, —SO₂NH₂, —PO₃H₂, —SO₂F, or CF₃;

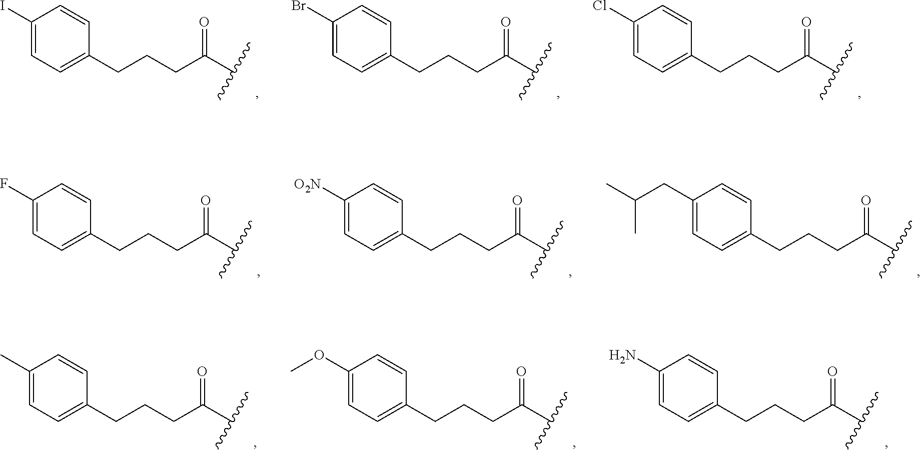

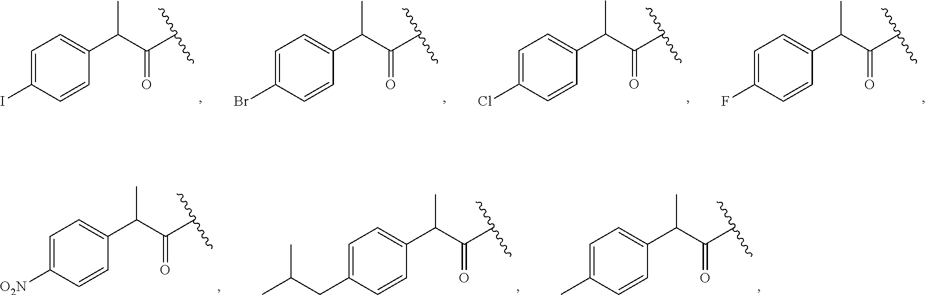

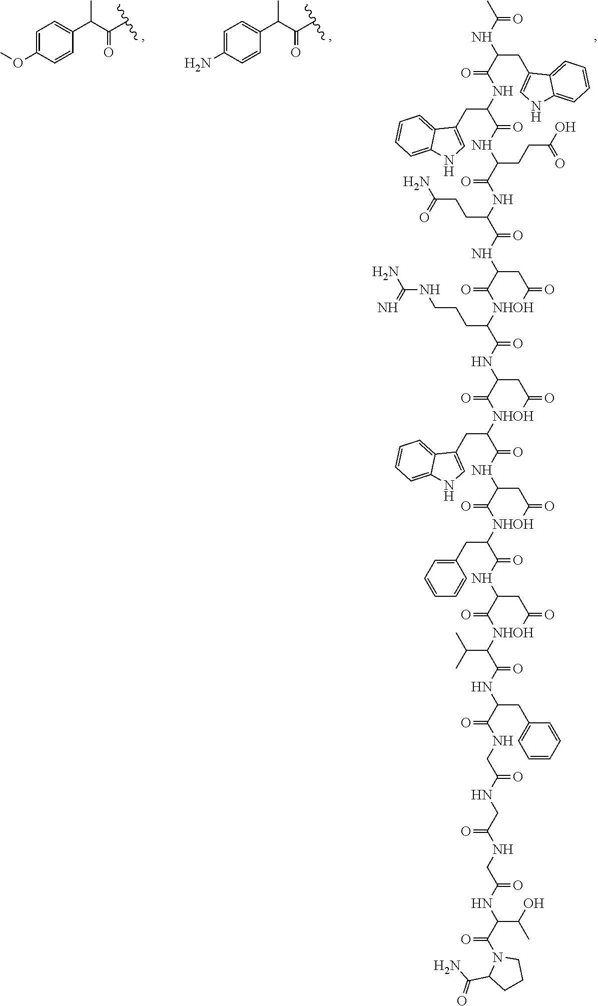 [SMILES: *C(=O)C(C)c1ccc(OC)cc1 ; *C(=O)C(C)c1ccc(N)cc1 ; CC(=O)NC(Cc1c[nH]c2ccccc12)C(=O)NC(Cc1c[nH]c2ccccc12)C(=O)NC(CCC(=O)O)C(=O)NC(CCC(N)=O)C(=O)NC(CC(=O)O)C(=O)NC(CCCNC(=N)N)C(=O)NC(CC(=O)O)C(=O)NC(Cc1c[nH]c2ccccc12)C(=O)NC(CC(=O)O)C(=O)NC(Cc1ccccc1)C(=O)NC(CC(=O)O)C(=O)NC(C(=O)NC(Cc1ccccc1)C(=O)NCC(=O)NCC(=O)NCC(=O)NC(C(=O)N1CCCC1C(N)=O)C(C)O)C(C)C]

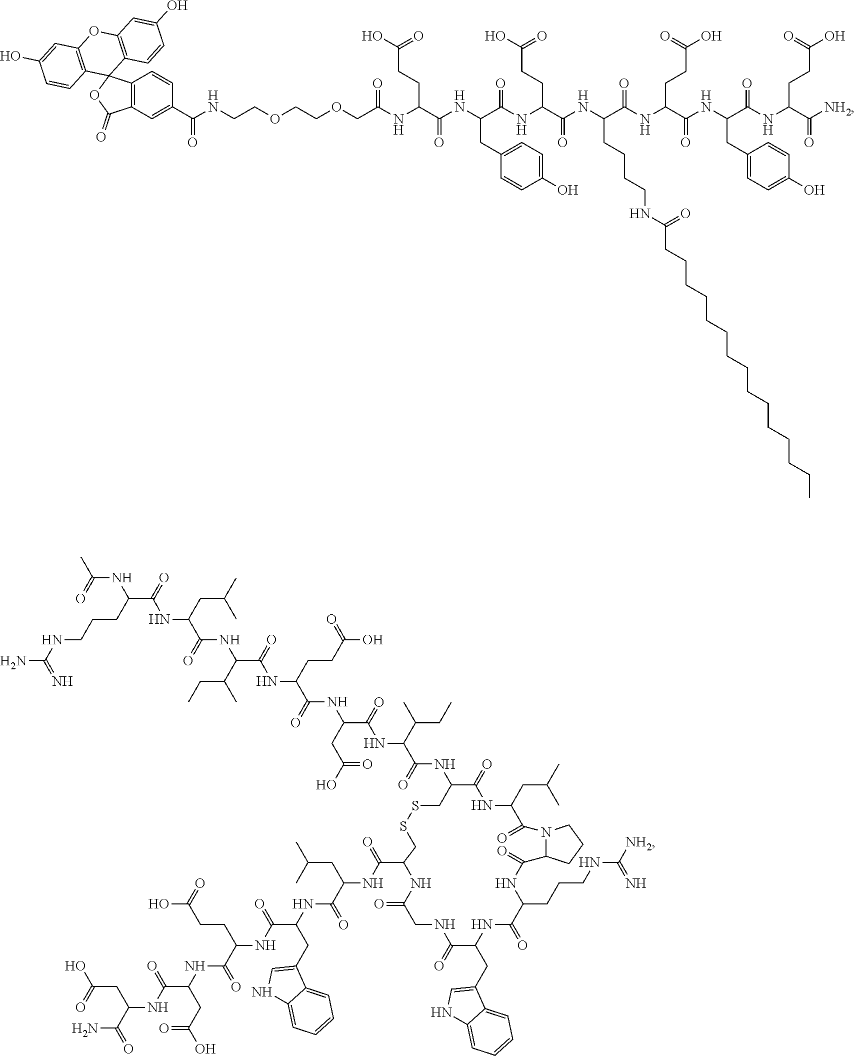

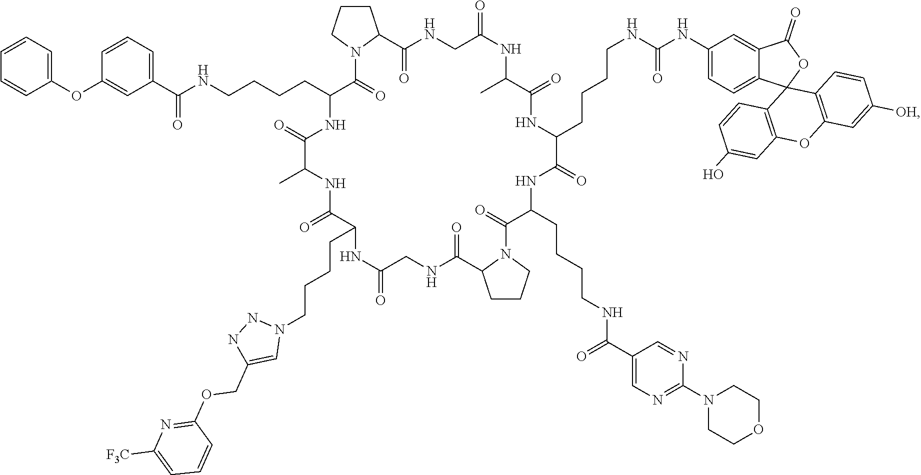 [SMILES: CC1NC(=O)CNC(=O)C2CCCN2C(=O)C(CCCCNC(=O)c2cccc(Oc3ccccc3)c2)NC(=O)C(C)NC(=O)C(CCCCN2C=C(COc3cccc(C(F)(F)F)n3)NN2)NC(=O)CNC(=O)C2CCCN2C(=O)C(CCCCNC(=O)c2cnc(N3CCOCC3)nc2)NC(=O)C(CCCCNC(=O)Nc2ccc3c(c2)C(=O)OC32c3ccc(O)cc3Oc3cc(O)ccc32)NC1=O]

ABD035, ABDCon, designed ankyrin repeat proteins (DARPins), dsFV CA645, nanobody (single-domain antibody (sdAb)), or a variable domain of new antigen receptor (VNAR) fused with anti-human serum albumin domain clone E06.

The BBMecL can comprise.
(i) Met-Val,
(ii) X-Lys or X-Arg, in which X=Gly or Arg, or a combination thereof,
(iii) Gly-Tyr,
(iv) Gly-Phe-(Lys),
(v) Gly-Pro,
(vi) Ala-X or Leu-X, wherein X is any amino acid, or a combination thereof,
(vii) Asp-X or Glu-X, wherein X is any amino acid, or a combination thereof,
(viii) γ-Glu-X, wherein X is any amino acid, and an acceptor peptide,
(ix) sucrose, maltose, trehalose, lactose, palatinose, or a combination of two or more of the foregoing,
(x) iodoinsulin B chain,
(xi) phlorizin,
(xii) p-nitrophenylphosphate, or
(xiii) a combination of two or more of the foregoing.

The AA can be an optical imaging agent, a radioactive imaging agent, or a radioactive therapeutic agent. The optical imaging agent can be a fluorescent dye. The fluorescent dye can be selected from the group consisting of S0456, fluorescein isothiocyanate (FITC), rhodamine, LS288, heptamethine cyanine dye (HMCD), SS180, acridine orange (AO), IRDye800CW, IR783, IR825, ZW800-1, or indocyanine green (ICG). Where AA is a radioactive imaging agent or radioactive therapeutic agent, AA can comprise a radioisotope selected from the group consisting of ¹⁸F, ⁴⁴Sc, ⁴⁷Sc, ⁵²Mn, ⁵⁵Co, ⁶⁴Cu, ⁶⁷Cu, ⁶⁷Ga, ⁶⁸Ga, ⁸⁶Y, ⁸⁹Zr, ⁹⁰Y, ^{99m}Tc, ¹¹¹In, ^{114m}In, ^{117m}Sn, ¹²⁴I, ¹²⁵I, ¹³¹I, ¹⁴⁹Tb, ¹⁵³Sm, ¹⁵²Tb, ¹⁵⁵Tb, ¹⁶¹Tb, ¹⁷⁷Lu, ¹⁸⁶Re, ¹⁸⁸Re, ²¹²Pb, ²¹²Bi, ²¹³Bi ²²³Ra, ²²⁴Ra, ²²⁵Ab, ²²⁵Ac, and ²²⁷Th.

The AA can comprise a radiolabeled prosthetic group comprising a radioisotope selected from the group consisting of ⁶⁸Ga, ¹⁸F, ⁹⁰Y ^{99m}Tc, ¹¹¹In, ¹⁷⁷Lu, ²²⁵Ac, ¹⁸P, ¹²⁴I, ¹²⁵I, ¹³¹I, and ²¹¹At. The radiolabeled prosthetic group can comprise a structure selected from:

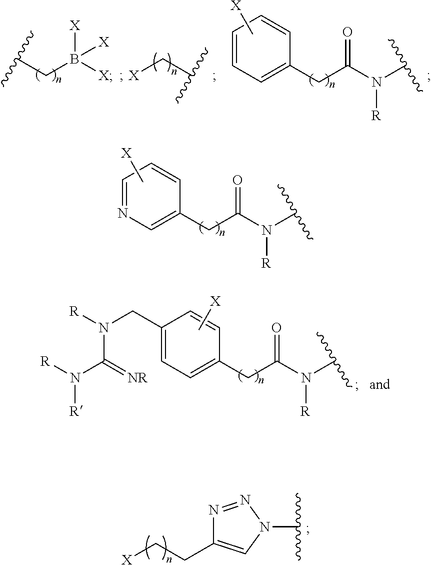

wherein R and R′ are independently hydrogen or methyl, and n is an integer selected from the group consisting of 0, 1, 2, 3, 4, 5, 6, 7, 8, 9, 10, 11, 12, 13, 14, 15, 16, 17, 18, 19 and 20. The radiolabeled prosthetic group can comprise a structure selected from:

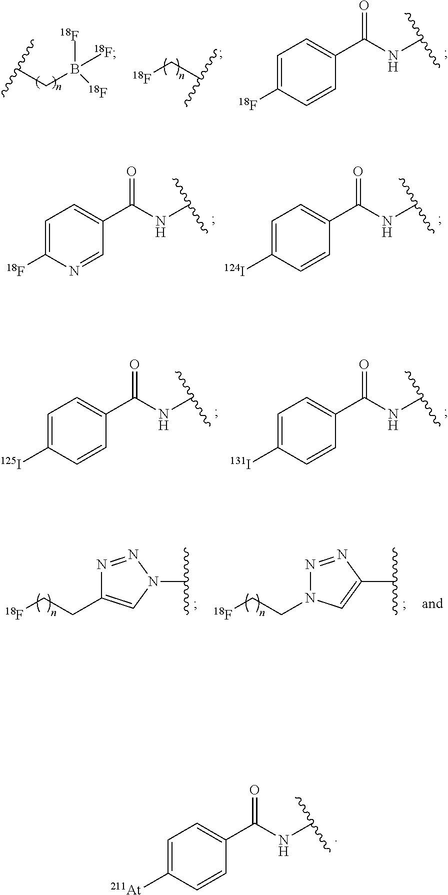

wherein n is an integer selected from the group consisting of 0, 1, 2, 3, 4, 5, 6, 7, 8, 9, 10, 11, 12, 13, 14, 15, 16, 17, 18, 19 and 20.

The AA of the conjugate can comprise a chelating agent selected from the group consisting of DOTA (1,4,7,10-tetraazacyclododecane-1,4,7,10-tetraacetic acid), TETA (1,4,8,11-tetraazacyclotetradecane-1,4,8,11-tetraacetic acid), SarAr (1-N-(4-Aminobenzyl)-3,6,10,13,16,19-hexaazabicyclo[6.6.6]-eicosane-1,8-diamine, NOTA (1,4,7-triazacyclononane-1,4,7-triacetic acid), NODAGA (2,2′-(7-(4-((2-aminoethyl)amino)-1-carboxy-4-oxobutyl)-1,4,7-triazonane-1,4-diyl)diacetic acid), HYNIC (6-Hydrazinonicotinic acid), NETA (4-[2-(bis-carboxymethylamino)-ethyl]-7-carboxymethyl-[1,4,7]triazonan-1-yl) acetic acid, TRAP (1,4,7-triazacyclononane-1,4,7-tris[methyl(2-carboxyethyl)phosphinic acid), HBED (N,N-bis(2-hydroxybenzyl)-ethylenediamine-N,N-diacetic acid), 2,3-HOPO (3-hydroxypyridin-2-one), PCTA (3,6,9,15-tetraazabicyclo[9.3.1]-pentadeca-1(15),11,13-triene-3,6,9,-triacetic acid), DFO (desferrioxamine), DTPA (diethylenetriaminepentaacetic acid), OCTAPA (N,N-bis(6-carboxy-2-pyridylmethyl)-ethylenediamine-N,N-diacetic acid), H2macropa (N,N′-bis[(6-carboxy-2-pyridipmethyl]-4,13-diaza-18-crown-6), H₂dedpa (1,2-[[carboxy)-pyridin-2-yl]-methylamino]ethane, EC20-head comprising β-l-diaminopropionic acid, aspartic acid, and cysteine, and a derivative of any of the foregoing. The AA can comprise a chelating agent selected from the group consisting of:

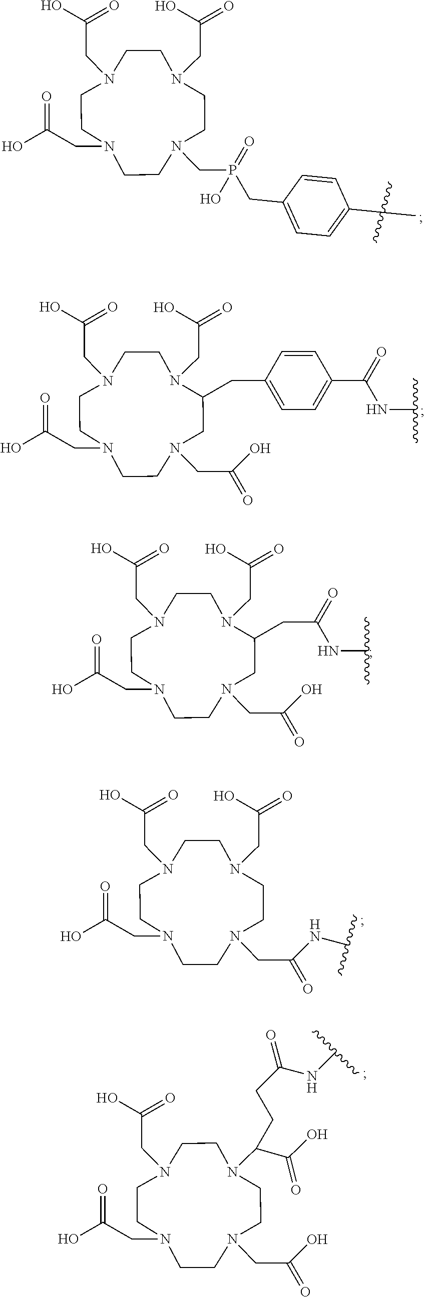 [SMILES: *c1ccc(CP(=O)(O)CN2CCN(CC(=O)O)CCN(CC(=O)O)CCN(CC(=O)O)CC2)cc1 ; *NC(=O)c1ccc(CC2CN(CC(=O)O)CCN(CC(=O)O)CCN(CC(=O)O)CCN2CC(=O)O)cc1 ; *NC(=O)CC1CN(CC(=O)O)CCN(CC(=O)O)CCN(CC(=O)O)CCN1CC(=O)O ; *NC(=O)CN1CCN(CC(=O)O)CCN(CC(=O)O)CCN(CC(=O)O)CC1 ; *NC(=O)CCC(C(=O)O)N1CCN(CC(=O)O)CCN(CC(=O)O)CCN(CC(=O)O)CC1]

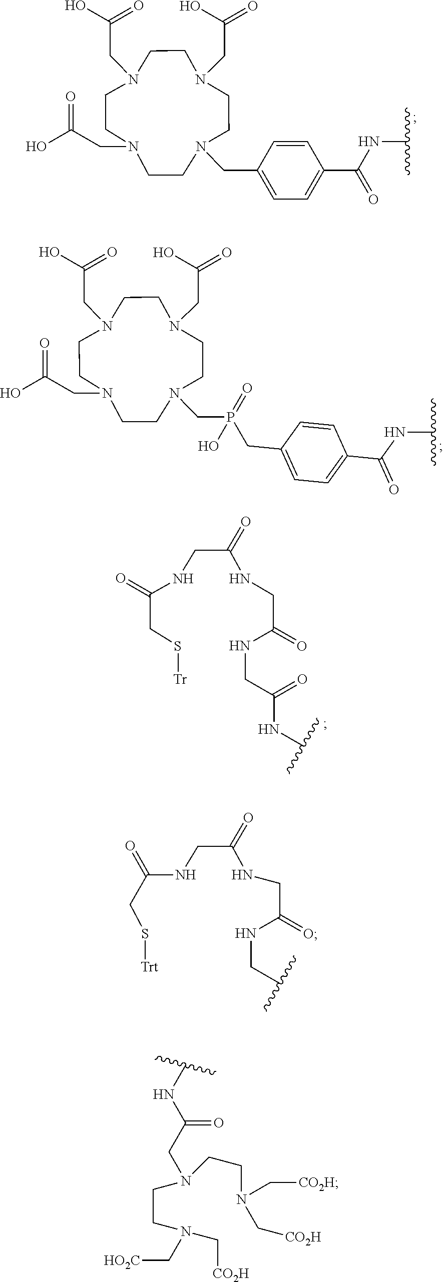

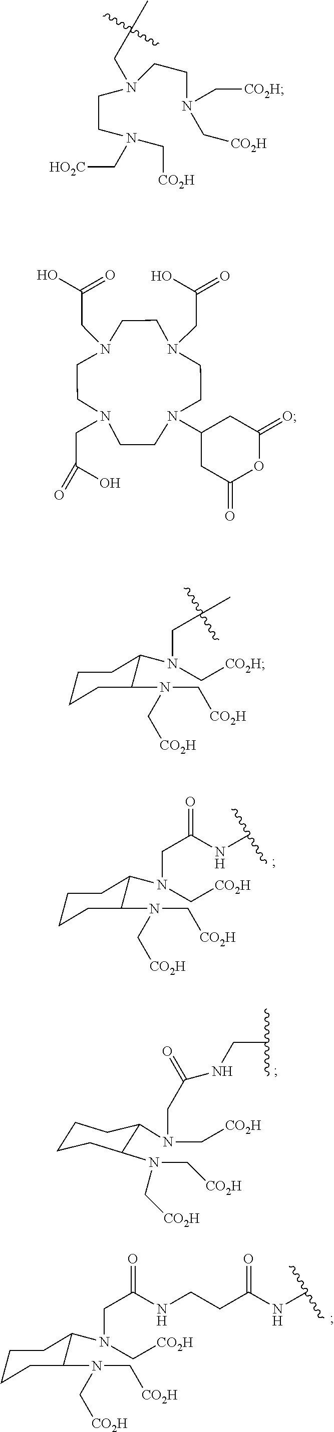 [SMILES: *CN(CCN(CC(=O)O)CC(=O)O)CCN(CC(=O)O)CC(=O)O ; O=C(O)CN1CCN(CC(=O)O)CCN(C2CC(=O)OC(=O)C2)CCN(CC(=O)O)CC1 ; *CN(CC(=O)O)C1CCCCC1N(CC(=O)O)CC(=O)O ; *NC(=O)CN(CC(=O)O)C1CCCCC1N(CC(=O)O)CC(=O)O ; *CNC(=O)CN(CC(=O)O)C1CCCCC1N(CC(=O)O)CC(=O)O ; *NC(=O)CCNC(=O)CN(CC(=O)O)C1CCCCC1N(CC(=O)O)CC(=O)O]

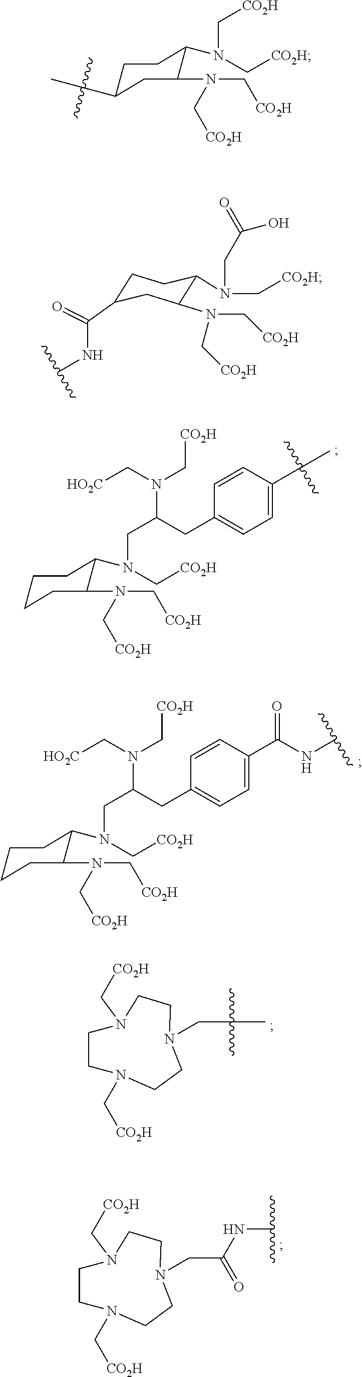 [SMILES: *C1CCC(N(CC(=O)O)CC(=O)O)C(N(CC(=O)O)CC(=O)O)C1 ; *NC(=O)C1CCC(N(CC(=O)O)CC(=O)O)C(N(CC(=O)O)CC(=O)O)C1 ; *c1ccc(CC(CN(CC(=O)O)C2CCCCC2N(CC(=O)O)CC(=O)O)N(CC(=O)O)CC(=O)O)cc1 ; *NC(=O)c1ccc(CC(CN(CC(=O)O)C2CCCCC2N(CC(=O)O)CC(=O)O)N(CC(=O)O)CC(=O)O)cc1 ; *CN1CCN(CC(=O)O)CCN(CC(=O)O)CC1 ; *NC(=O)CN1CCN(CC(=O)O)CCN(CC(=O)O)CC1]

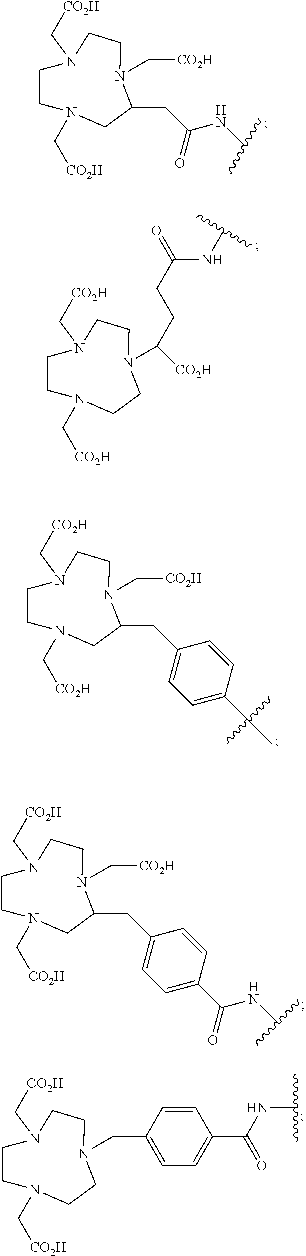 [SMILES: *NC(=O)CC1CN(CC(=O)O)CCN(CC(=O)O)CCN1CC(=O)O ; *NC(=O)CCC(C(=O)O)N1CCN(CC(=O)O)CCN(CC(=O)O)CC1 ; *c1ccc(CC2CN(CC(=O)O)CCN(CC(=O)O)CCN2CC(=O)O)cc1 ; *NC(=O)c1ccc(CC2CN(CC(=O)O)CCN(CC(=O)O)CCN2CC(=O)O)cc1 ; *NC(=O)c1ccc(CN2CCN(CC(=O)O)CCN(CC(=O)O)CC2)cc1]

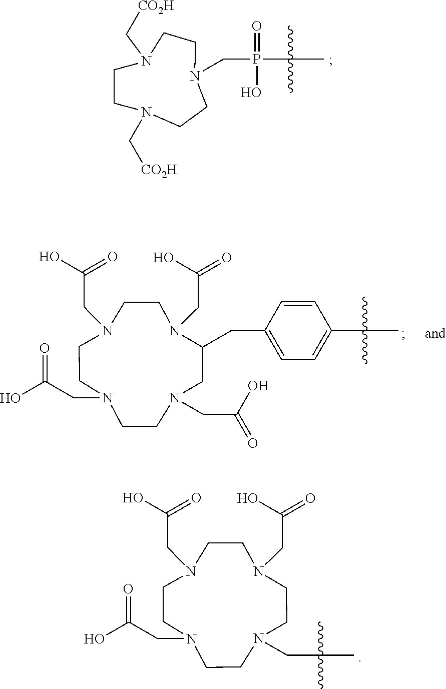 [SMILES: *P(=O)(O)CN1CCN(CC(=O)O)CCN(CC(=O)O)CC1 ; *c1ccc(CC2CN(CC(=O)O)CCN(CC(=O)O)CCN(CC(=O)O)CCN2CC(=O)O)cc1 ; *CN1CCN(CC(=O)O)CCN(CC(=O)O)CCN(CC(=O)O)CC1]

The conjugate can have the structure:

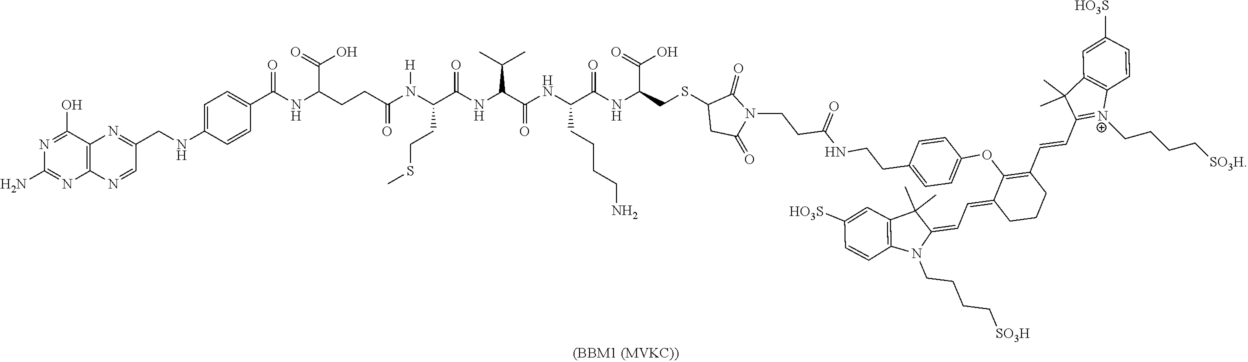

The conjugate can have the structure:

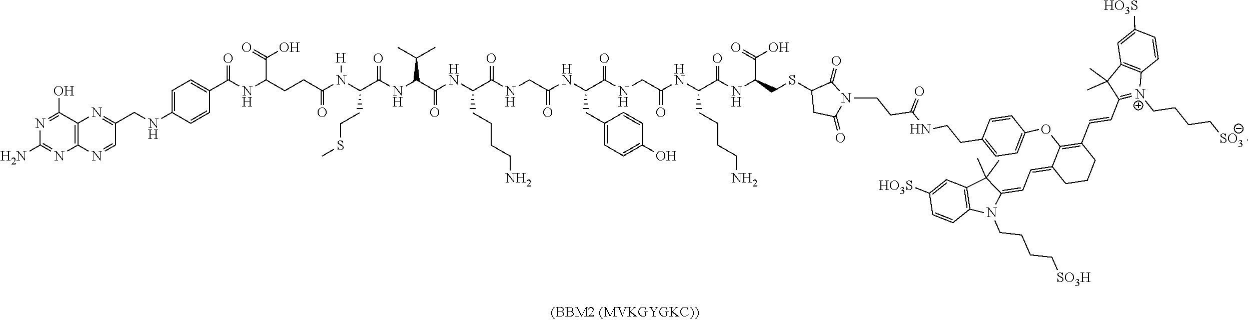

The conjugate can have the structure:

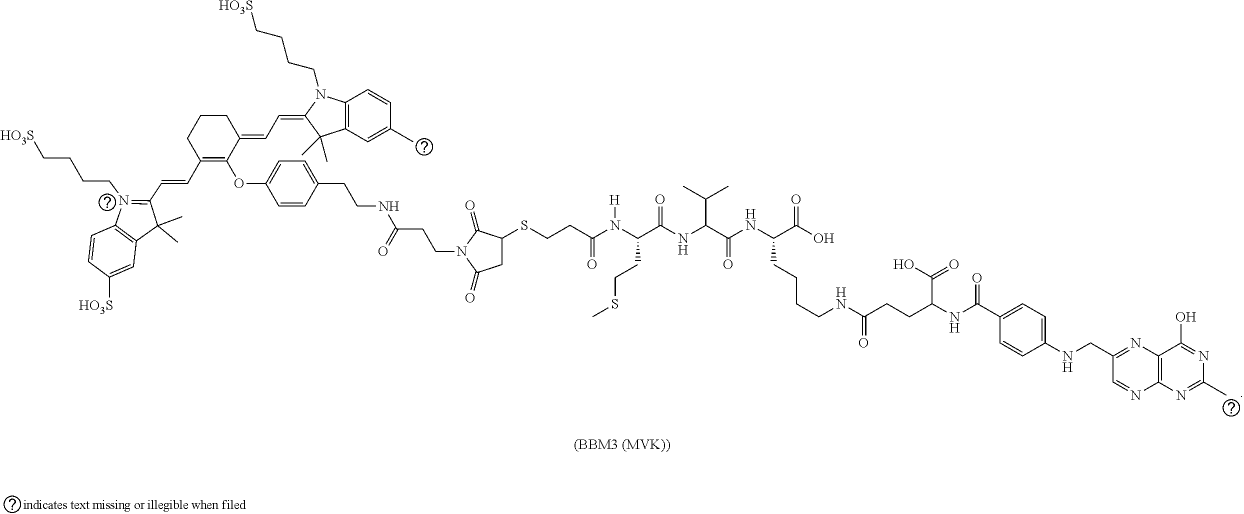

The conjugate can have the structure:

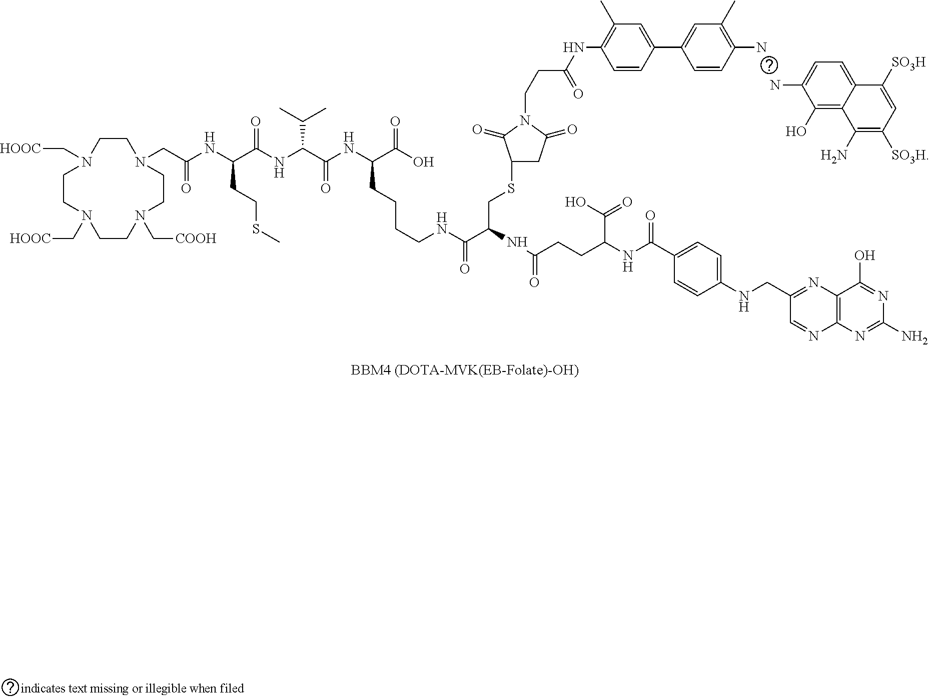 [SMILES: CSCC[C@@H](NC(=O)CN1CCN(CC(=O)O)CCN(CC(=O)O)CCN(CC(=O)O)CC1)C(=O)N[C@@H](C(=O)N[C@H](CCCCNC(=O)[C@@H](CSC1CC(=O)N(CCC(=O)Nc2ccc(-c3ccc(N)c(C)c3)cc2C)C1=O)NC(=O)CCC(NC(=O)c1ccc(NCc2cnc3nc(N)nc(O)c3n2)cc1)C(=O)O)C(=O)O)C(C)C ; Nc1ccc2c(S(=O)(=O)O)cc(S(=O)(=O)O)c(N)c2c1O]

The conjugate can have the structure:

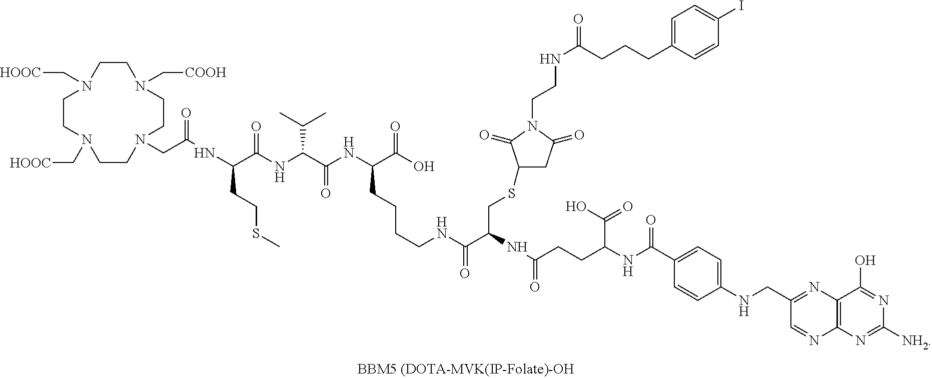 [SMILES: CSCC[C@@H](NC(=O)CN1CCN(CC(=O)O)CCN(CC(=O)O)CCN(CC(=O)O)CC1)C(=O)N[C@@H](C(=O)N[C@H](CCCCNC(=O)[C@@H](CSC1CC(=O)N(CCNC(=O)CCCc2ccc(I)cc2)C1=O)NC(=O)CCC(NC(=O)c1ccc(NCc2cnc3nc(N)nc(O)c3n2)cc1)C(=O)O)C(=O)O)C(C)C]

The conjugate can have the structure:

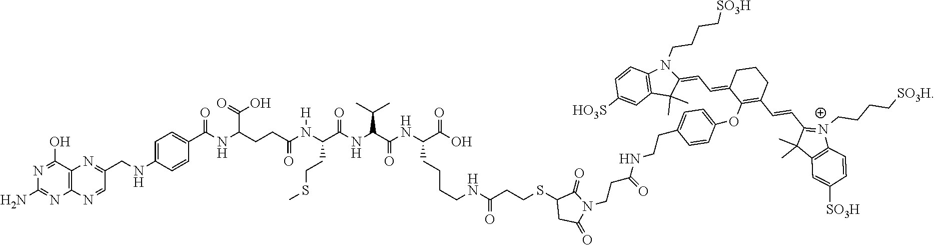

The conjugate can have the structure:

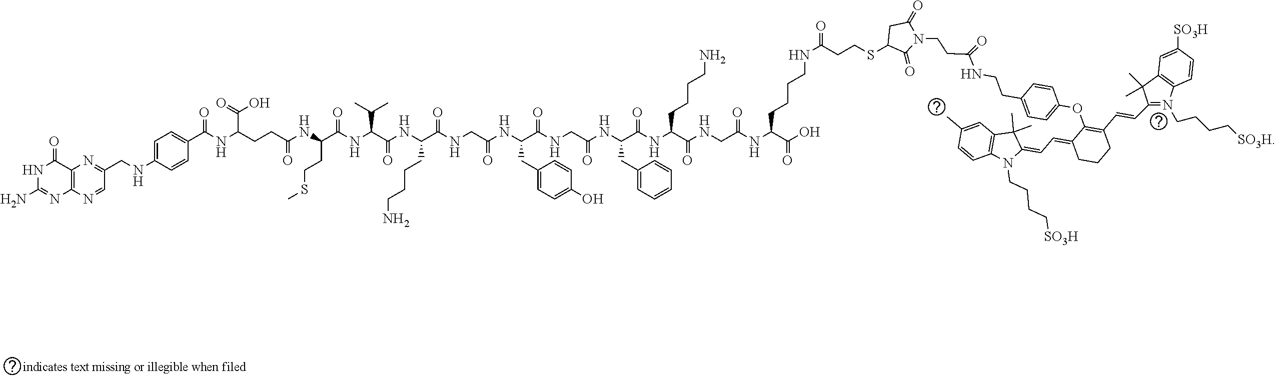

The conjugate can have the structure:

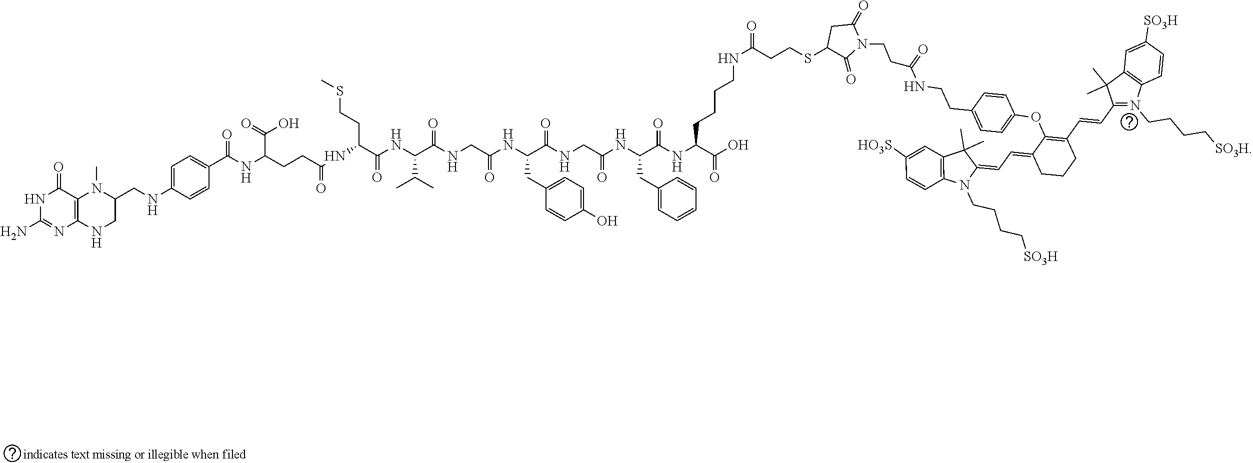

The conjugate can have the structure:

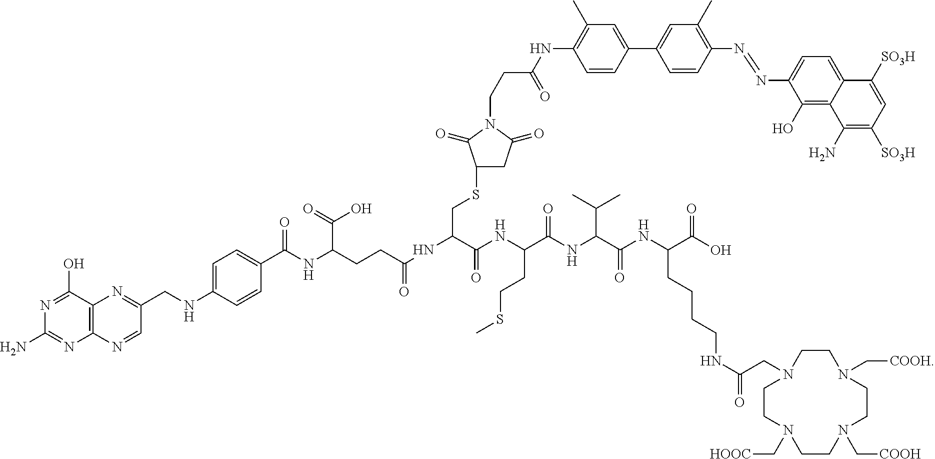 [SMILES: CSCCC(NC(=O)C(CSC1CC(=O)N(CCC(=O)Nc2ccc(-c3ccc(/N=N/c4ccc5c(S(=O)(=O)O)cc(S(=O)(=O)O)c(N)c5c4O)c(C)c3)cc2C)C1=O)NC(=O)CCC(NC(=O)c1ccc(NCc2cnc3nc(N)nc(O)c3n2)cc1)C(=O)O)C(=O)NC(C(=O)NC(CCCCNC(=O)CN1CCN(CC(=O)O)CCN(CC(=O)O)CCN(CC(=O)O)CC1)C(=O)O)C(C)C]

The conjugate can have the structure:

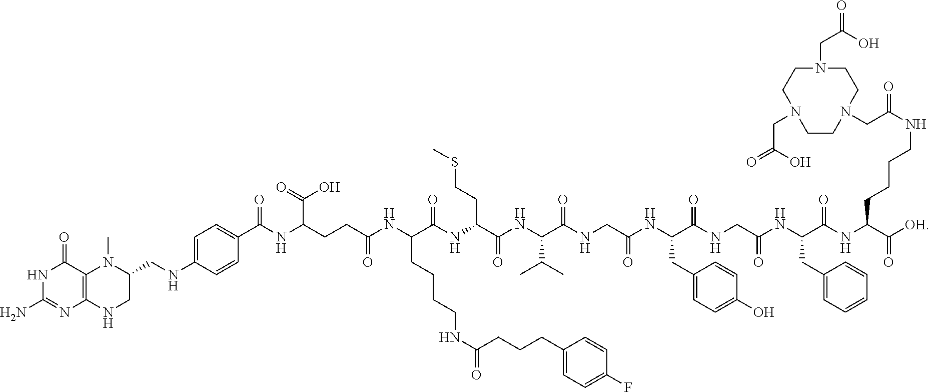 [SMILES: CSCC[C@@H](NC(=O)C(CCCCNC(=O)CCCc1ccc(F)cc1)NC(=O)CCC(NC(=O)c1ccc(NC[C@@H]2CNc3nc(N)[nH]c(=O)c3N2C)cc1)C(=O)O)C(=O)N[C@H](C(=O)NCC(=O)N[C@@H](Cc1ccc(O)cc1)C(=O)NCC(=O)N[C@@H](Cc1ccccc1)C(=O)N[C@@H](CCCCNC(=O)CN1CCN(CC(=O)O)CCN(CC(=O)O)CC1)C(=O)O)C(C)C]

The conjugate can have the structure:

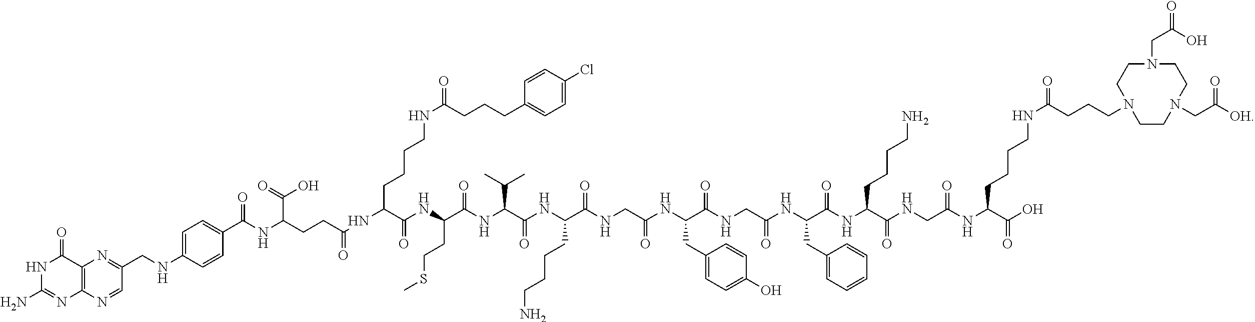 [SMILES: CSCC[C@@H](NC(=O)C(CCCCNC(=O)CCCc1ccc(Cl)cc1)NC(=O)CCC(NC(=O)c1ccc(NCc2cnc3nc(N)[nH]c(=O)c3n2)cc1)C(=O)O)C(=O)N[C@H](C(=O)N[C@@H](CCCCN)C(=O)NCC(=O)N[C@@H](Cc1ccc(O)cc1)C(=O)NCC(=O)N[C@@H](Cc1ccccc1)C(=O)N[C@@H](CCCCN)C(=O)NCC(=O)N[C@@H](CCCCNC(=O)CCCN1CCN(CC(=O)O)CCN(CC(=O)O)CC1)C(=O)O)C(C)C]

Further provided is a composition comprising a conjugate and a pharmaceutically acceptable carrier.

Still further provided is a method of imaging, treating, or imaging and treating a tumor in a subject by targeting radioactivity, alone or in further combination with an optical imaging agent, to cells of the tumor, macrophages associated with the tumor (e.g., tumor-associated macrophages (TAMs)), or both, which method comprises administering to the subject an effective amount of (i) a conjugate or (ii) a composition comprising the conjugate and a pharmaceutically acceptable carrier. The method can further comprise imaging the tumor. The imaging can be by optical imaging, positron emission tomography (PET), or single photon emission computed tomography (SPECT).

Kits for imaging, treating, or imaging and treating a tumor in a subject are also provide. In certain embodiments, a kit comprises at least one dosage unit of a conjugate or a composition in a first container, and at least one dosage of a second active agent or composition comprising the second active agent. In certain embodiments, the at least one dosage unit of the second active agent or composition comprising the second active agent is contained in the first container. Alternatively, the at least one dosage unit of the second active agent or composition comprising the second active agent is contained in a second container. The kit can also comprise a means for administration of the conjugate or the composition (e.g., a syringe, a stent, a cannula, a trocar, or the like).

### DESCRIPTION OF THE FIGURES

FIG. 1A shows flow cytometry results for validation of folate-targeting for OTL38.

FIG. 1B shows flow cytometry results for validation of folate-targeting for BBM1.

FIG. 1C shows flow cytometry results for validation of folate-targeting for BBM2.

FIG. 2A shows the folate conjugate binding curve for OTL38 (K_{d}=6.7 nM).

FIG. 2B shows the folate conjugate binding curve for BBM1 (K_{d}=12.3 nM).

FIG. 2C shows the folate conjugate binding curve for BBM2 (K_{d}=29.6 nM).

FIG. 2D shows the folate conjugate binding curve for BBM3 (Kd=16.0 nM).

FIGS. 3A and 3B show the results of fluorescence scanning of organs of mice housed on a folate-deficient diet for 3+ weeks, injected with 1 nmol of OTL38, BBM1 or BBM2 and sacrificed 24 hours later, with FIG. 3A showing the results of short exposure and FIG. 3B showing the results of high exposure (high fluorescence shown in FIG. 3A further identified by a white circle).

FIGS. 4A and 4B show the results of fluorescence scanning of organs of mice housed on a folate-deficient diet for 3+ weeks, injected with 1 nmol of OTL38, BBM1 or BBM2 and sacrificed 1 hour later (high fluorescence further identified by a white rectangle).

FIGS. 5A and 5B show the results of fluorescence scanning of organs of mice housed on a folate-deficient diet for 3+ weeks, injected with 1 nmol of OTL38, BBM1 or BBM2 and sacrificed 3 hours later (high fluorescence further identified by white rectangles).

FIGS. 6A and 6B show the results of fluorescence scanning of organs of mice housed on a folate-deficient diet for 3+ weeks, injected with 1 nmol of OTL38, BBM1 or BBM2 and sacrificed 6 hours later (high fluorescence further identified by white rectangles).

FIG. 7 shows the results of fluorescence scanning of mice housed on a folate-deficient diet for 3+ weeks, injected with 1 nmol of OTL38 or BBM3 and scanned for fluorescence 1 hour (1 h p.i.) or 3 hours (3 h p.i.) post-injection (fluorescence further indicated by white circle).

FIG. 8A shows the results of fluorescence scanning of organs of mice housed on a folate-deficient diet for 3+ weeks, injected with 1 nmol of OTL38 or BBM3 and sacrificed 3 hours later (highest fluorescence further indicated by black rectangles).

FIG. 8B shows the results of fluorescence scanning of organs of mice housed on a folate-deficient diet for 3+ weeks, injected with 1 nmol of OTL38, BBM1, or BBM2 and sacrificed 3 hours later (highest fluorescence further indicated by a white rectangle).

FIG. 9A shows the results of scanning of mice housed on a folate-deficient diet for 3+ weeks, injected with 5 nmol of OTL38 or BBM3 and scanned for fluorescence 1 hour (1 h p.i.) or 3 hours (3 h p.i.) post-injection (highest fluorescence further indicated by black circles).

FIG. 9B shows the results of scanning of mice housed on a folate-deficient diet for 3+ weeks, injected with 5 nmol of OTL38 or BBM3 and scanned for fluorescence 6 hours (6 h p.i.) or 24 hours (24 h p.i.) post-injection (highest fluorescence further indicated by black circles).

FIG. 10 shows the results of scanning of organs of mice housed on a folate-deficient diet for 3+ weeks, injected with 5 nmol of OTL38 or BBM3 and sacrificed 24 hours later (highest fluorescence further indicated by a black rectangle).

FIG. 11 shows the results of scanning of organs of athymic nude mice housed on a folate-deficient diet for 3+ weeks, injected with 5 nmol of OTL38, BBM1 or BBM2 and sacrificed 24 hours later.

FIG. 12 shows the results of scanning of athymic nude mice housed on a folate-deficient diet for 3+ weeks, injected with 5 nmol of OTL38 or BBM3 (1-2 mice/conjugate), and scanned for fluorescence in the tumor (MDA-MD-231) vs. the kidney at 1 hour (1 hour p.i.), 3 hours (3 hours p.i.), 6 hours (6 hours p.i.), and 24 hours post-injection (24 hours p.i.) (fluorescence further indicated by black circles).

FIG. 13 shows the results of scanning of athymic nude mice housed on a folate-deficient diet for 3+ weeks, tail vein-injected with 5 nmol of OTL38 or BBM3 (1-2 mice/conjugate), and scanned for fluorescence in the tumor (MDA-MB-231) vs. the kidney at 1 hour (1 hour p.i.), 3 hours (3 hours p.i.), 6 hours (6 hours p.i.), and 24 hours post-injection (24 hours p.i.) (fluorescence further indicated by black circles).

FIG. 14 shows the results of scanning of organs of athymic nude mice housed on a folate-deficient diet for 3+ weeks, tail vein-injected with 5 nmol of OTL38 or BBM3 (1-2 mice/conjugate), and scanned for fluorescence in the tumor, heart, lungs, liver, spleen and kidneys 24 hours post-injection.

FIG. 15 is a radio-chromatogram of ¹¹¹In-BBM4.

FIG. 16 is a radio-chromatogram of ¹¹¹In-BBM5.

FIG. 17A is a graph of concentration (nM) vs. CPM, which shows the binding of Indium-111 radiolabeled BBM4 conjugates in KB cells.

FIG. 17B is a graph of concentration (nM) vs. CPM, which shows the binding of Indium-111 radiolabeled BBM5 conjugates in KB cells.

FIG. 18 shows the SPECT/CT images of Indium-111 radiolabeled BBM4 conjugates in a healthy mouse. 1 nmol injected radiolabeled with ˜250 uCi of In-111. Yellow arrows indicate kidneys.

FIG. 19 shows the SPECT/CT images of Indium-111 radiolabeled BBM5 conjugates in a healthy mouse. 1 nmol injected radiolabeled with ˜250 uCi of In-111. Yellow arrows indicate kidneys.

FIG. 20 shows the SPECT/CT images of Indium-111 radiolabeled BBM4 conjugates in a healthy mouse. 5 nmol injected radiolabeled with ˜250 uCi of In-111. Yellow arrows indicate kidneys.

FIG. 21 is a radio-chromatogram of ¹⁷⁷Lu-BBM4.

FIG. 22 is a radio-chromatogram of ¹⁷⁷Lu-BBM5.

FIG. 23A is a graph of day vs. tumor volume (mm³), which shows the gross volume of KB tumors in mice treated with ¹⁷⁷Lu radiolabeled BBM conjugates.

FIG. 23B is a graph of day vs. relative tumor size, which shows the relative size of KB tumors in mice treated with ¹⁷⁷Lu radiolabeled BBM conjugates.

FIG. 23C is a graph of day vs. relative body weight (%), which shows the relative body weights of mice with KB tumors and treated with ¹⁷⁷Lu radiolabeled BBM conjugates.

FIG. 24 shows the SPECT/CT images of a mouse treated with ¹⁷⁷Lu radiolabeled BBM4 conjugate. 5 nmol injected radiolabeled with ˜500 uCi of Lu-177. White arrow indicates tumor, yellow arrow indicates kidney.

FIG. 25 shows the SPECT/CT images of a mouse treated with ¹⁷⁷Lu radiolabeled BBM5 conjugate. 5 nmol injected radiolabeled with ˜500 uCi of Lu-177. White arrow indicates tumor, yellow arrow indicates kidney.

### DETAILED DESCRIPTION

For the purposes of promoting an understanding of the principles of the present disclosure, reference will now be made to the embodiments illustrated in the drawings and specific language will be used to describe the same. It will nevertheless be understood that no limitation of scope is intended by the description of these embodiments. On the contrary, this disclosure is intended to cover alternatives, modifications, and equivalents as may be included within the spirit and scope of this disclosure.

The present disclosure is predicated, at least in part, on the discovery that the use of a kidney brush border membrane (BBM) enzyme substrate to link a folate-receptor targeting ligand with an agent for imaging or radiotherapy allows rapid clearance of the conjugate from the kidneys. Rapid clearance enables renal carcinoma imaging and minimizes renal toxicity in radiotherapy of multiple tumors. In addition to rapid clearance from the kidneys, the conjugates enable specific targeting, high tumor penetration, and rapid clearance from receptor-negative tissues. Further, since folate is expressed on cancer-associated macrophages of many solid tumors, the conjugates can be used to treat the stroma of many types of cancer.

Provided is a conjugate of formula I or formula II:

FRTL-BBMecL-AA (formula I)

or

FRTL-Alb-BBMecL-AA (formula II),

wherein:
FRTL is a folate receptor-targeting ligand,
BBMecL is a kidney brush border membrane (BBM) enzyme-cleavable linker,
Alb is an albumin-binder moiety, and
AA is an active agent.
   In certain embodiments, the FRTL has a molecular weight below 10,000. The Alb can associate noncovalently with serum albumin.

The FRTL can have the structure:

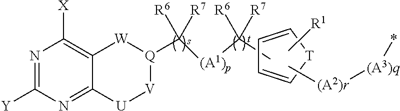

in which:
T is selected from the group consisting of S, O, NR⁴b, and —HC═CH—,
U, V, and W represent divalent moieties, each independently selected from the group consisting of —(R^{6a})C═, —N═, —(R^{6a})C(R^{7a})—, and —N(R^{4a})—, wherein R^{6a} and R^{7a} are each independently selected from the group consisting of hydrogen, halo, and C₁-C₁₂ alkoxy; or R^{6a} and R^{7a} are taken together to form a carbonyl group,
X and Y are each independently selected from the group consisting of halo, R², OR², SR³, and NR⁴R⁵,
Q is C or CH,
A¹ and A² are each independently selected from the group consisting of oxygen, sulfur, —C(Z)—, —C(Z)O—, —OC(Z)—, —N(R^{4b})—, —C(Z)N(R^{4b})—, —N(R^{4b})C(Z)—, —OC(Z)N(R^{4b})—, —N(R^{4b})C(Z)O—, —N(R^{4b})C(Z)N(R^{5b})—, —S(O)—, —S(O)₂—, —N(R^{4b})S(O)₂—, —C(R^{6b})(R^{7b})—, —N(C≡CH)—, —N(CH₂C≡CH)—, C₁-C₁₂ alkylene, and C₁-C₁₂ alkyleneoxy, wherein Z is oxygen or sulfur,
R², R³, R⁴, R^{4a}, R^{4b}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂alkynyl, (C₁-C₁₂ alkoxy)carbonyl, and (C₁-C₁₂ alkylamino)carbonyl,
A³ is an amino acid,
R¹ is selected from the group consisting of hydrogen, halo, C₁-C₁₂ heteroalkyl, and C₁-C₁₂ alkoxy,
R⁶ and R⁷ are each independently selected from the group consisting of hydrogen, halo, C₁-C₁₂ alkyl, and C₁-C₁₂ alkoxy; or R⁶ and R⁷ are taken together to form a carbonyl group,
q is an integer from 1 to 3,
p, r, s, and t are each independently 0 or 1, and
* indicates the point of attachment to the BBMecL or the Alb.

“C₁-C₁₂ alkyl” refers to a straight, branched, or cyclic hydrocarbon chain containing from 1 to 12 carbon atoms. Representative examples of C₁-C₁₂ alkyl groups in accordance with the present teachings include, but are not limited to, methyl, ethyl, propyl, iso-propyl, cyclopropyl, butyl, iso-butyl, tert-butyl, sec-butyl, cyclobutyl, pentyl, cyclopentyl, iso-pentyl, neopentyl, hexyl, cyclohexyl, iso-hexyl, neohexyl, heptyl, cycloheptyl, iso-heptyl, neoheptyl, octyl, cyclooctyl, iso-octyl, neooctyl, nonyl, cyclononyl, iso-nonyl, neononyl, decyl, cyclodecyl, iso-decyl, neodecyl, undecyl, cycloundecyl, iso-undecyl, neoundecyl, dodecyl, cyclododecyl, iso-dodecyl, and neododecyl.

“C₁-C₁₂ alkoxy” is a C₁-C₁₂ alkyl singularly bonded to an oxygen.

“C₁-C₁₂ alkanoyl” is a C₁-C₁₂ alkyl singularly bonded to a carbonyl.

“C₁-C₁₂ alkenyl” is a C₁-C₁₂ alkyl comprising a C═C.

“C₁-C₁₂ alkynyl” is a C₁-C₁₂ alkyl comprising a C≡C.

“(C₁-C₁₂ alkoxy)carbonyl” is a C₁-C₁₂ alkoxy bonded to a carbonyl.

“(C₁-C₁₂ alkylamino)carbonyl” is a C₁-C₁₂ alkylamino (i.e., a C₁-C₁₂ alkyl bonded to an amino) bonded to a carbonyl.

“C₁-C₁₂ heteroalkyl” is a C₁-C₁₂ alkyl comprising at least one heteroatom (i.e., an atom other than carbon or hydrogen).

“Halogen” and “halo” refer to fluorine, chlorine, iodine, or bromine.

In certain embodiments, Q is CH. In certain embodiments, X is OH and Y is NH₂.

In certain embodiments, W and U are —N(R^{4a})—, Q is CH, V is CH₂, A¹ is —N(R^{4b})—, s is 1, p is 1, and t is 0 (e.g., A¹ is directly connected to the heterocycle). In certain embodiments, R^{4a} and R^{4b} are each independently alkyl or heteroalkyl. In certain embodiments, R^{4a} and R^{4b} are each methyl.

The FRTL can have the structure:

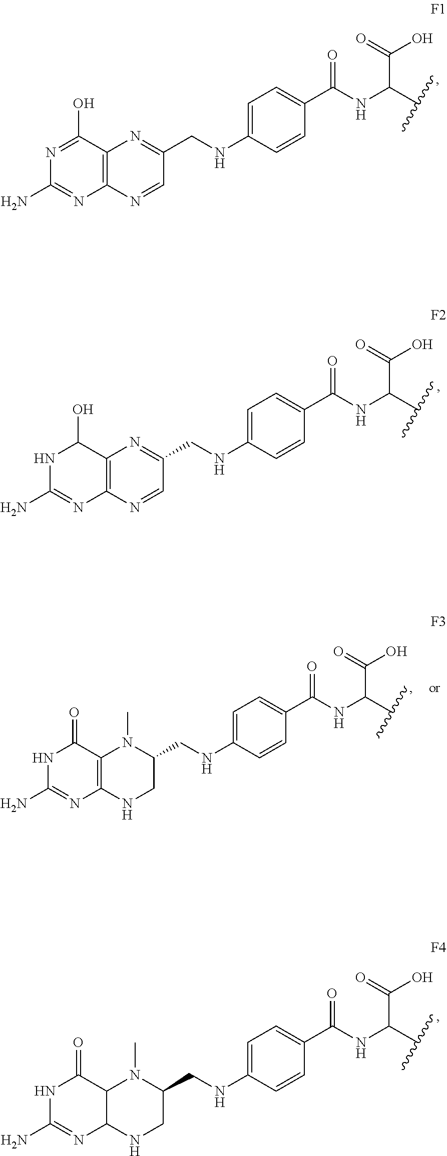

wherein

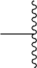

Indicates a point of attachment of FRTL to Alb or BBMecL.

Alb can have the structure:

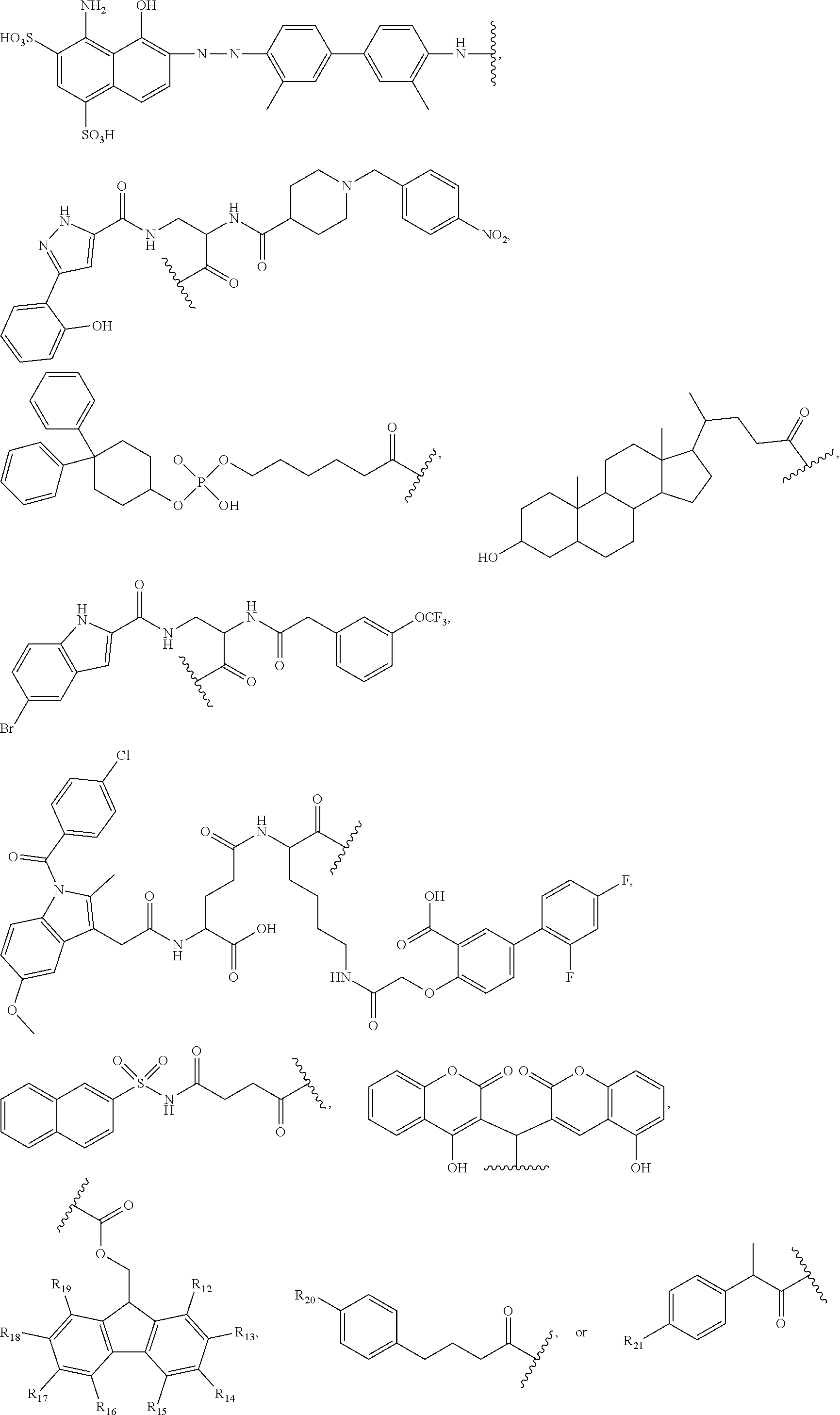

wherein

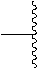

indicates a point of attachment of Alb to FRTL,
R₁₂₋₁₉ are independently —H, —C₁-C₆ alkyl, —F, —Cl, —Br, —I, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, or —SO₂F; and R₂₀ and R₂₁ are independently —H, —C₁-C₆ alkyl, —F, —Cl, —Br, —I, —O—C₁₋₆ alkyl, —CN, —CHO, —B(OH)₂, —C═C—C(O)aryl, —C═C—S(O)₂aryl, —CO₂H, —SO₃H, —SO₂NH₂, —PO₃H₂, —SO₂F, or CF₃;

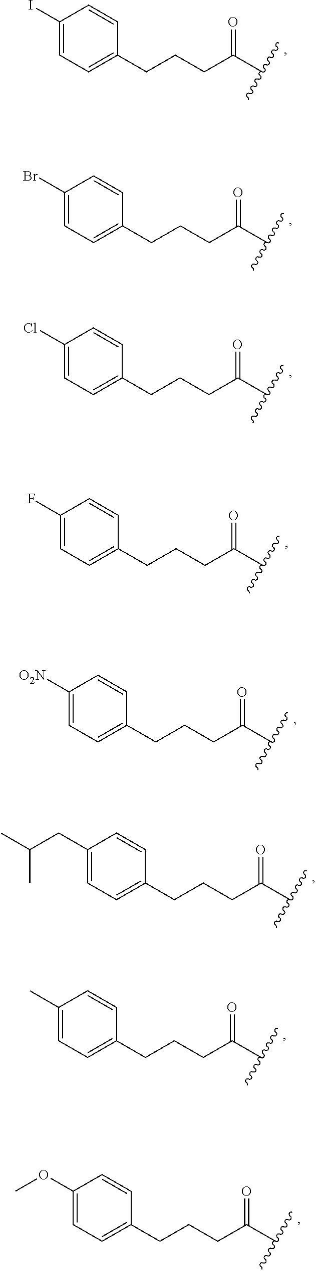

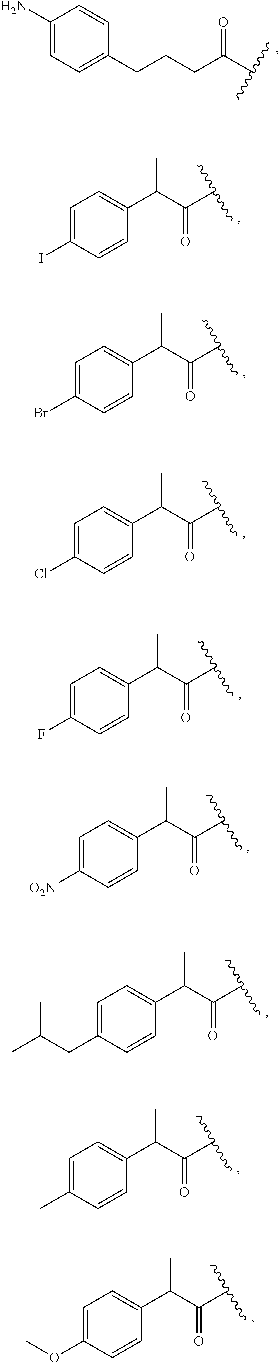

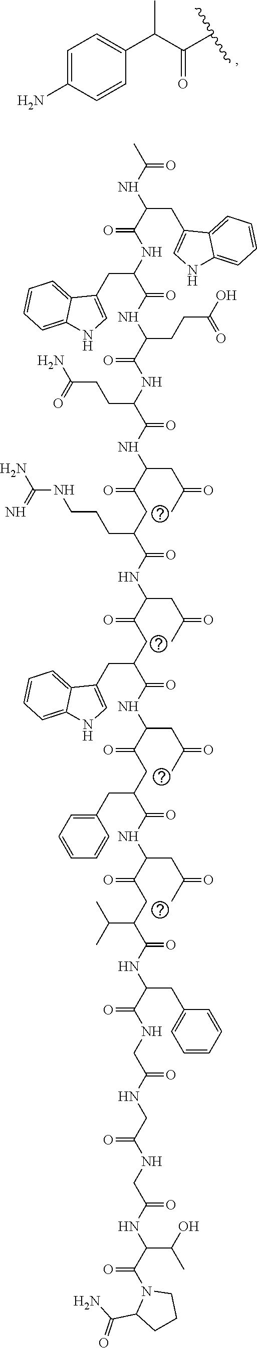 [SMILES: *C(=O)C(C)c1ccc(N)cc1 ; CC(=O)CC(NC(=O)C(CCCNC(=N)N)CC(=O)C(CC(C)=O)NC(=O)C(CCC(N)=O)NC(=O)C(CCC(=O)O)NC(=O)C(Cc1c[nH]c2ccccc12)NC(=O)C(Cc1c[nH]c2ccccc12)NC(C)=O)C(=O)CC(Cc1c[nH]c2ccccc12)C(=O)NC(CC(C)=O)C(=O)CC(Cc1ccccc1)C(=O)NC(CC(C)=O)C(=O)CC(C(=O)NC(Cc1ccccc1)C(=O)NCC(=O)NCC(=O)NCC(=O)NC(C(=O)N1CCCC1C(N)=O)C(C)O)C(C)C]

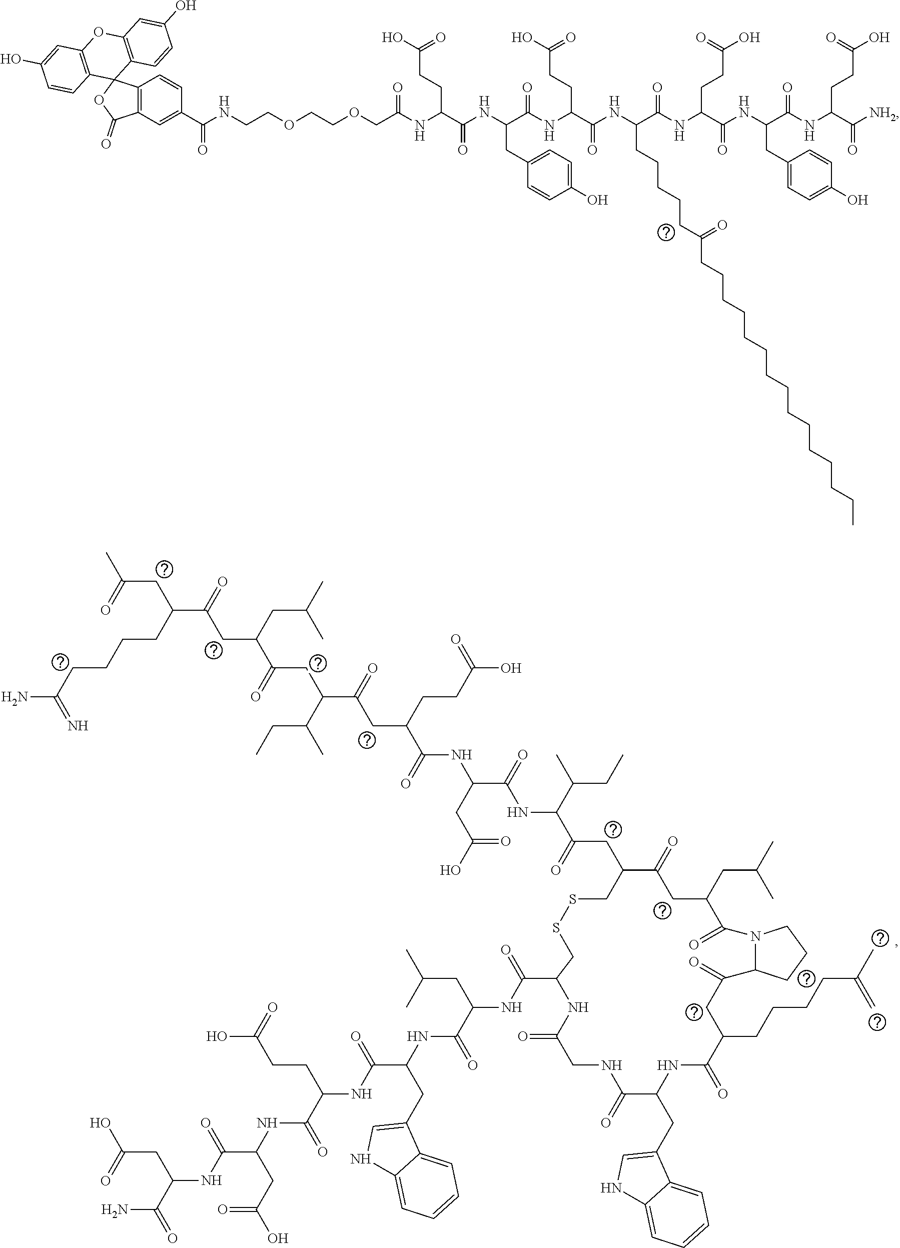

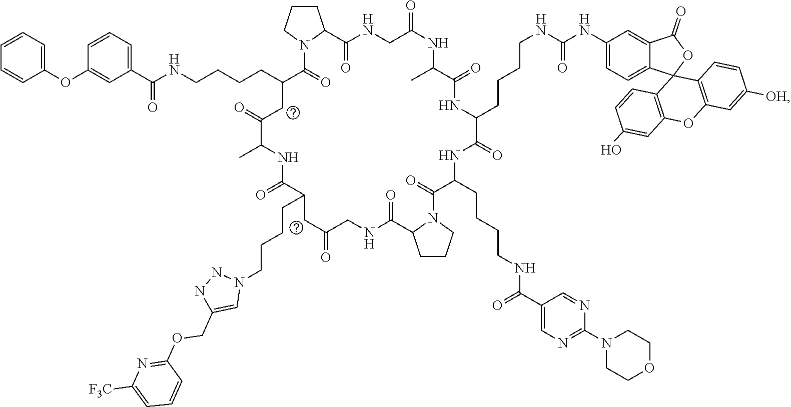 [SMILES: CC1NC(=O)C(CCCCN2C=C(COc3cccc(C(F)(F)F)n3)NN2)CC(=O)CNC(=O)C2CCCN2C(=O)C(CCCCNC(=O)c2cnc(N3CCOCC3)nc2)NC(=O)C(CCCCNC(=O)Nc2ccc3c(c2)C(=O)OC32c3ccc(O)cc3Oc3cc(O)ccc32)NC(=O)C(C)NC(=O)CNC(=O)C2CCCN2C(=O)C(CCCCNC(=O)c2cccc(Oc3ccccc3)c2)CC1=O]

ABD035 (Jonsson et al., Protein Eng. Des. Sel. 21, 515-527. doi: 10.1093/protein/gzn028 (2008)), ABDCon (Jacobs et al., Protein Eng Des Sel 28(10): 385-393 (October 2015), designed ankyrin repeat proteins (DARPins), dsFV CA645 (Zorzi et al., Med Chem Comm 10: 1068-1081 (2019)), a nanobody (single-domain antibody (sdAb)), or a variable domain of new antigen receptor (VNAR) fused with anti-human serum albumin domain clone E06 (Barelle et al., Antibodies 4(3): 240-258 (2015)).

The BBMecL of the conjugate can be any suitable BBM enzyme-cleavable substrate (e.g., a linker). Examples include those shown in Table 1; however, it will be appreciated that the BBMecL can comprise any suitable BBM enzyme-cleavable substrate now known or hereinafter developed.

TABLE 1
Brush Border Membrane Enzyme Cleavable Substrates

| SUBSTRATE | BBM ENZYME |
|---|---|
| Met-Val | Neprilysin/neutral |
| | endopeptidase (NEP) |
| X-Lys or X-Arg, wherein | Carboxypeptidase M (CPM) |
| X = Gly or Arg | |
| Gly-Tyr | NEP, CPM, Dipeptidase |
| | 1 (DPEP1) |
| Gly-Phe-(Lys) | NEP |
| Gly-Pro | Dipeptidyl-peptidase 4 (DPP4), |
| | Fibroblast Activation |
| | Protein (FAP) |
| Ala-X or Leu-X, wherein X = | Aminopeptidase N |
| any amino acid | |
| Asp-X or Glu-X, wherein X = | Aminopeptidase A |
| any amino acid | |
| γ-Glu-X, wherein X = any | γ-glutamyltransferase |
| amino acid, and an acceptor peptide | |
| sucrose | Sucrase-isomaltase |
| maltose | Maltase (glucoamylase) |
| trehalose | trehalase |
| lactose | Lactose-glycosyl-ceramidase |
| palatinose | Sucrase-isomaltase |
| Iodoinsulin B chain | Neprilysin/NEP |
| phlorizin | Lactose-glycosyl-ceramidase |
| p-nitrophenylphosphate | Alkaline phosphatase |

In certain embodiments, the BBMecL of the conjugate comprises:
(i) methionine-valine (Met-Val),
(ii) X-lysine (X-Lys) or X-arginine (X-Arg) or a combination thereof, in which X is glycine (Gly) or arginine (Arg),
(iii) glycine-tyrosine (Gly-Tyr),
(iv) glysine-phenylalinine-(lysine) (Gly-Phe-(Lys)),
(v) glycine-proline (Gly-Pro),
(vi) alanine-X (Ala-X) or leucine-X (Leu-X) or a combination thereof, wherein X is any amino acid,
(vii) aspartic acid-X (Asp-X) or glutamic acid-X (Glu-X) or a combination thereof, wherein X is any amino acid,
(viii) γ-glutamic acid-X (γ-Glu-X), wherein X is any amino acid, and an acceptor peptide,
(ix) sucrose, maltose, trehalose, lactose, palatinose, or a combination of two or more of the foregoing,
(x) iodoinsulin B chain,
(xi) phlorizin,
(xii) p-nitrophenylphosphate, or
(xiii) a combination of two or more of the foregoing.

In certain embodiments, the BBMecL of the conjugate comprises a kidney BBM cleavable substrate/linker. In certain embodiments, the BBM cleavable linker of the BBMecL is attached to at least the AA of the conjugate and the FRTL (e.g., a conjugate of formula I). In certain embodiments, the BBM cleavable linker of the BBMecL is attached to at least the AA of the conjugate and the Alb (e.g., a conjugate of formula II). When such embodiments of the conjugate are administered to a subject (e.g., systemically), the BBM linker can cleave and release the AA from the remainder of the conjugate (e.g., the FRTL and/or the Alb). Accordingly, even if a portion of the conjugate can bind to a folate receptor (e.g., FRα) on a brush border membrane—for example, of the subject's kidneys—the remainder of the conjugate is released and, thus, not taken up or retained within the organ (e.g., kidney).

The BBM linker can, in certain embodiments, include one or more unnatural amino acids (UAAs) including, without limitation, one of more of a D-amino acid, citrulline, hydroxyproline, norleucine, 3-nitrotyrosine, nitroarginine, naphtylalanine, aminobutyric acid (Abu), 2, 4-diaminobutyric acid (DAB), methionine sulfoxide, methionine sulfone, and the like.

It should be appreciated that such physiological conditions resulting in a BBM cleavable linker breaking include standard chemical hydrolysis reactions that occur, for example, at physiological pH, or as a result of compartmentalization into a cellular organelle such as an endosome having a lower pH than cytosolic pH. Illustratively, the BBM cleavable linkers described herein can undergo cleavage under other physiological or metabolic conditions.

The AA can be an optical imaging agent, a radioactive imaging agent, or a radioactive therapeutic agent.

In certain embodiments, AA is an optical imaging agent. The optical imaging agent can be any compound (or a radical thereof) that emits a detectable signal (e.g., an electromagnetic signal (e.g., a radio signal, a fluorescent signal, gamma rays) or a mass). Examples of optical imaging agents include, but are not limited to, a radio-imaging agent (e.g., a positron emission tomography (PET) imaging agent or a single photon emission computed tomography (SPECT) imaging agent), a fluorescent imaging agent (e.g., a fluorescent dye), or the like. The imaging agent can be a magnetic resonance (MR) agent. In some embodiments, AA comprises (e.g., a radical of) a radiolabeled functional group suitable for PET imaging, SPECT imaging, other radio-imaging techniques, magnetic resonance imaging, or radiotherapy. AA can comprise a radical of a radio-imaging, radiotherapeutic, or magnetic resonance isotope.

In certain embodiments, the optical imaging agent is an optical imaging agent comprising a fluorescent dye. The fluorescent dye can be selected from the group consisting of 50456, fluorescein isothiocyanate (FITC), rhodamine, LS288, heptamethine cyanine dye (HMCD), SS180, acridine orange (AO), IRDye800CW, IR783, IR825, ZW800-1, or indocyanine green (ICG).

In certain embodiments, the AA is a radioactive imaging agent or radioactive therapeutic agent comprising a radioisotope selected from the group consisting of ¹⁸F, ⁴⁴Sc, ⁴⁷Sc, ⁵²Mn ⁵⁵Co ⁶⁴Cu, ⁶⁷Cu, ⁶⁷Ga, ⁶⁸Ga, ⁸⁶Y, ⁸⁹Zr, ⁹⁰Y, ^{99m}Tc, ¹¹¹In, ^{114m}In, ^{117m}Sn, ¹²⁴I, ¹²⁵I, ¹³¹I, ¹⁴⁹Tb, ¹⁵³Sm, ¹⁵²Tb, ¹⁵⁵Tb, ¹⁶¹Tb, ¹⁷⁷Lu, ¹⁸⁶Re, ¹⁸⁸Re, ²¹²Pb, ²¹²Bi, ²¹³Bi, ²²³Ra, ²²⁴Ra, ²²⁵Ab, ²²⁵Ac, and ²²⁷Th.

In certain embodiments, the AA comprises a radiolabeled prosthetic group comprising a radioisotope selected from the group consisting of ⁶⁸Ga, ¹⁸F, ⁹⁰Y, ^{99m}Tc, ¹¹¹In, ¹⁷⁷Lu, ²²⁵Ac, ¹⁸P, ¹²⁴I, ¹²⁵I, ¹³¹I, and ²¹¹At. The radiolabeled prosthetic group can comprise a structure selected from:

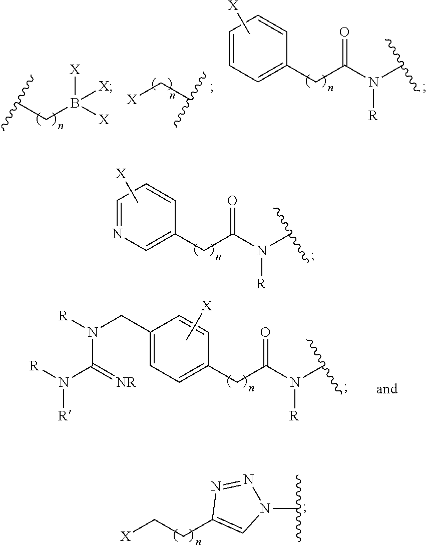

wherein R and R′ are independently hydrogen or methyl, and n is an integer selected from the group consisting of 0, 1, 2, 3, 4, 5, 6, 7, 8, 9, 10, 11, 12, 13, 14, 15, 16, 17, 18, 19 and 20. The radiolabeled prosthetic group can comprise a structure selected from:

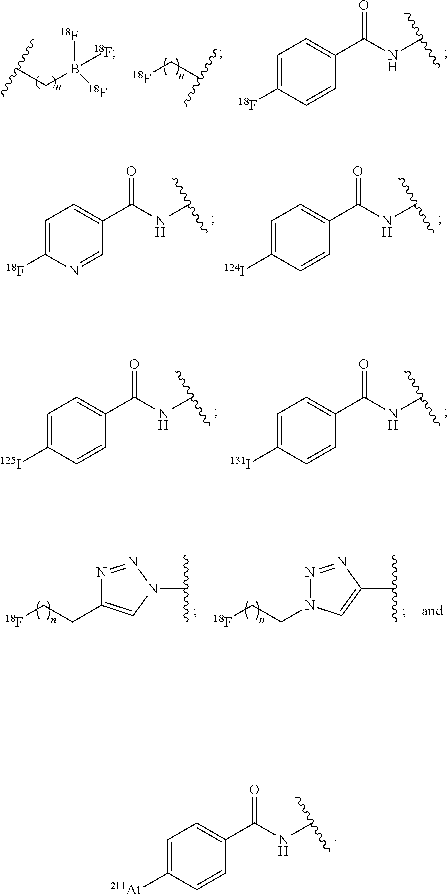

wherein n is an integer selected from the group consisting of 0, 1, 2, 3, 4, 5, 6, 7, 8, 9, 10, 11, 12, 13, 14, 15, 16, 17, 18, 19 and 20.

AA can be a chelating group (e.g., a chelating agent (or a radical thereof)). “Chelating group” refers to a polydentate chemical group which can bind to a central metal atom with multiple binding interactions by using two or more binding sites on the chelating group. The combination of chelating group and metal atom is a chelate. The binding of the chelating group to the metal atom can be by non-covalent interactions or bonding; in some embodiments the binding of a chelating group to a metal atom is by multiple coordinate bonds.

The AA can comprise a chelating group or agent selected from the group consisting of DOTA (1,4,7,10-tetraazacyclododecane-1,4,7,10-tetraacetic acid), TETA (1,4,8,11-tetraazacyclotetradecane-1,4,8,11-tetraacetic acid), SarAr (1-N-(4-Aminobenzyl)-3,6,10,13,16,19-hexaazabicyclo[6.6.6]-eicosane-1,8-diamine, NOTA (1,4,7-triazacyclononane-1,4,7-triacetic acid), NODAGA (2,2′-(7-(4-((2-aminoethyl)amino)-1-carboxy-4-oxobutyl)-1,4,7-triazonane-1,4-diyl)diacetic acid), HYNIC (6-Hydrazinonicotinic acid), NETA (4-[2-(bis-carboxymethylamino)-ethyl]-7-carboxymethyl-[1,4,7]triazonan-1-yl) acetic acid, TRAP (1,4,7-triazacyclononane-1,4,7-tris[methyl(2-carboxyethyl)phosphinic acid), HBED (N,N-bis(2-hydroxybenzyl)-ethylenediamine-N,N-diacetic acid), 2,3-HOPO (3-hydroxypyridin-2-one), PCTA (3,6,9,15-tetraazabicyclo[9.3.1]-pentadeca-1(15),11,13-triene-3,6,9,-triacetic acid), DFO (desferrioxamine), DTPA (diethylenetriaminepentaacetic acid), OCTAPA (N,N-bis(6-carboxy-2-pyridylmethyl)-ethylenediamine-N,N-diacetic acid), H₂macropa (N,N′-bis[(6-carboxy-2-pyridipmethyl]-4,13-diaza-18-crown-6), H₂dedpa (1,2-[[carboxy)-pyridin-2-yl]-methylamino]ethane, EC20-head comprising β-l-diaminopropionic acid, aspartic acid, ethylenediarminetetraacetic acid (EDTA), and cysteine, and a derivative of any of the foregoing. In certain embodiments, the AA can comprise a radical of any of the foregoing chelating agents.

In certain embodiments, AA can be or comprise a radical of a group covalently bound to an isotope (or metal) suitable for radio-imaging, radiotherapy, or magnetic resonance imaging.

Representative chelating groups include, but are not limited to (including free bases thereof, such as wherein a proton (H+) of one or more CO₂H (COOH) is removed to form COO—):

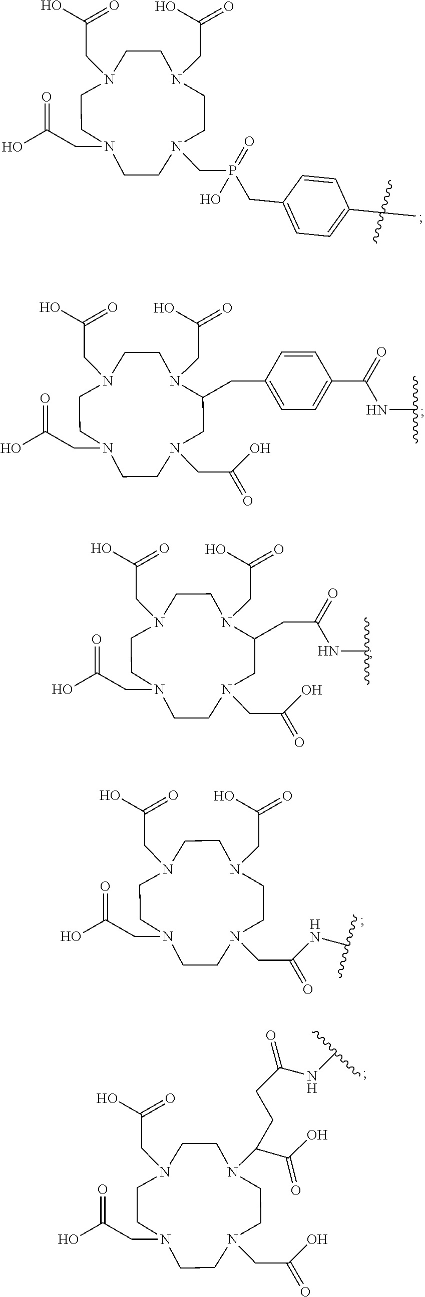 [SMILES: *c1ccc(CP(=O)(O)CN2CCN(CC(=O)O)CCN(CC(=O)O)CCN(CC(=O)O)CC2)cc1 ; *NC(=O)c1ccc(CC2CN(CC(=O)O)CCN(CC(=O)O)CCN(CC(=O)O)CCN2CC(=O)O)cc1 ; *NC(=O)CC1CN(CC(=O)O)CCN(CC(=O)O)CCN(CC(=O)O)CCN1CC(=O)O ; *NC(=O)CN1CCN(CC(=O)O)CCN(CC(=O)O)CCN(CC(=O)O)CC1 ; *NC(=O)CCC(C(=O)O)N1CCN(CC(=O)O)CCN(CC(=O)O)CCN(CC(=O)O)CC1]

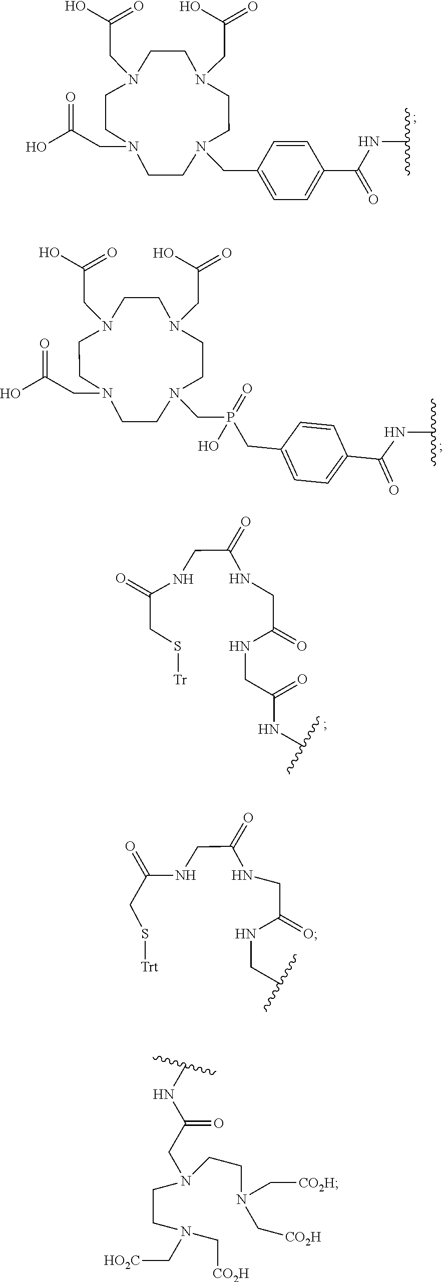

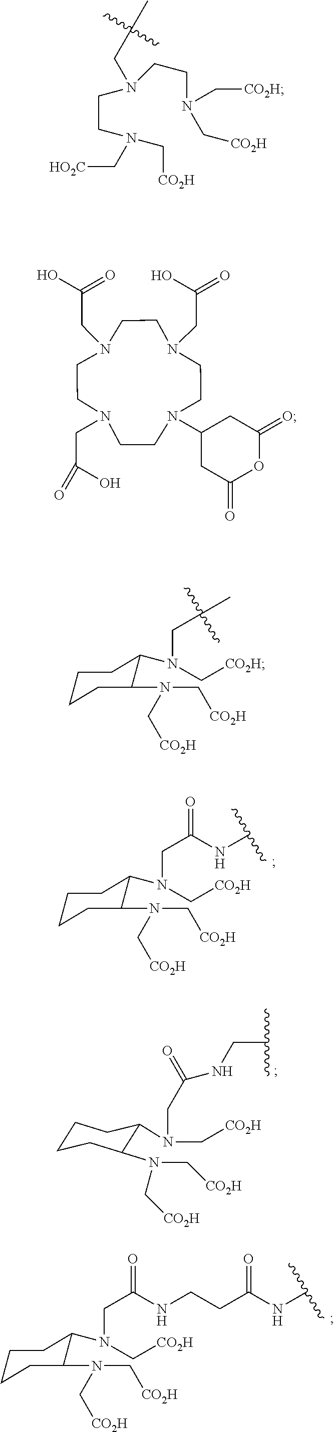 [SMILES: *CN(CCN(CC(=O)O)CC(=O)O)CCN(CC(=O)O)CC(=O)O ; O=C(O)CN1CCN(CC(=O)O)CCN(C2CC(=O)OC(=O)C2)CCN(CC(=O)O)CC1 ; *CN(CC(=O)O)C1CCCCC1N(CC(=O)O)CC(=O)O ; *NC(=O)CN(CC(=O)O)C1CCCCC1N(CC(=O)O)CC(=O)O ; *CNC(=O)CN(CC(=O)O)C1CCCCC1N(CC(=O)O)CC(=O)O ; *NC(=O)CCNC(=O)CN(CC(=O)O)C1CCCCC1N(CC(=O)O)CC(=O)O]

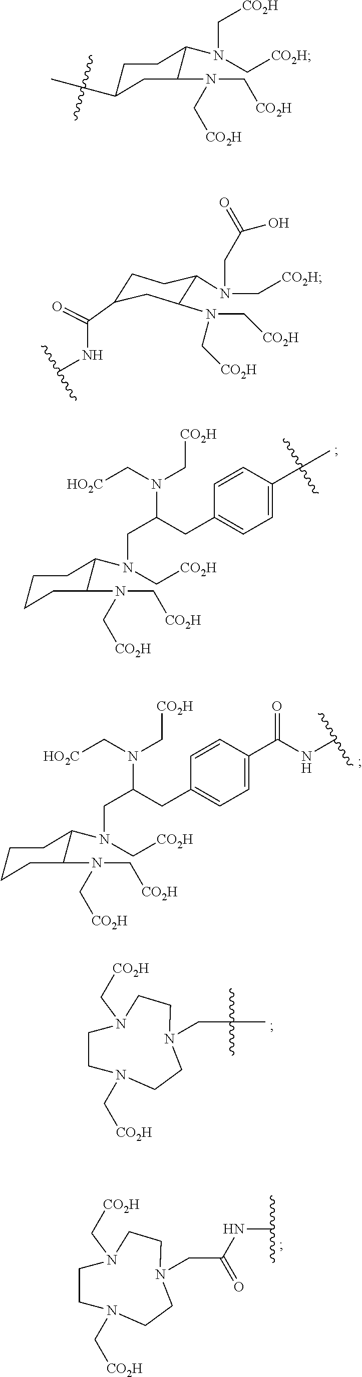 [SMILES: *C1CCC(N(CC(=O)O)CC(=O)O)C(N(CC(=O)O)CC(=O)O)C1 ; *NC(=O)C1CCC(N(CC(=O)O)CC(=O)O)C(N(CC(=O)O)CC(=O)O)C1 ; *c1ccc(CC(CN(CC(=O)O)C2CCCCC2N(CC(=O)O)CC(=O)O)N(CC(=O)O)CC(=O)O)cc1 ; *NC(=O)c1ccc(CC(CN(CC(=O)O)C2CCCCC2N(CC(=O)O)CC(=O)O)N(CC(=O)O)CC(=O)O)cc1 ; *CN1CCN(CC(=O)O)CCN(CC(=O)O)CC1 ; *NC(=O)CN1CCN(CC(=O)O)CCN(CC(=O)O)CC1]

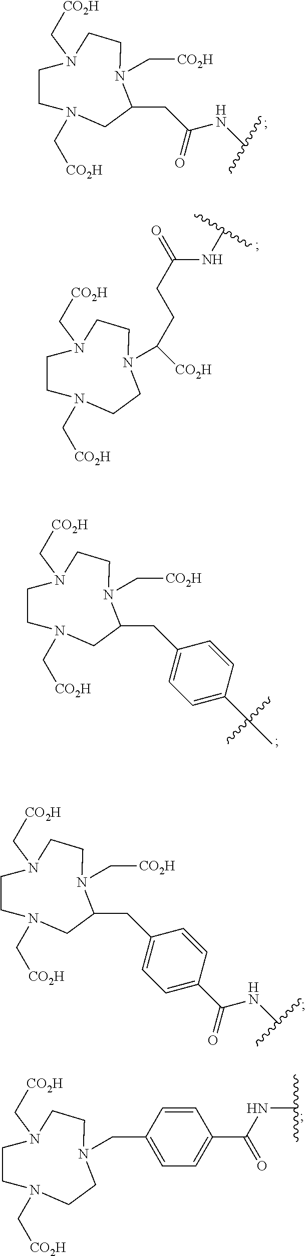 [SMILES: *NC(=O)CC1CN(CC(=O)O)CCN(CC(=O)O)CCN1CC(=O)O ; *NC(=O)CCC(C(=O)O)N1CCN(CC(=O)O)CCN(CC(=O)O)CC1 ; *c1ccc(CC2CN(CC(=O)O)CCN(CC(=O)O)CCN2CC(=O)O)cc1 ; *NC(=O)c1ccc(CC2CN(CC(=O)O)CCN(CC(=O)O)CCN2CC(=O)O)cc1 ; *NC(=O)c1ccc(CN2CCN(CC(=O)O)CCN(CC(=O)O)CC2)cc1]

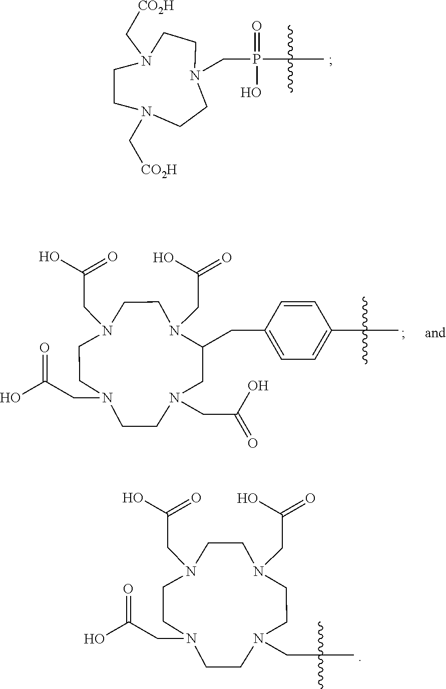 [SMILES: *P(=O)(O)CN1CCN(CC(=O)O)CCN(CC(=O)O)CC1 ; *c1ccc(CC2CN(CC(=O)O)CCN(CC(=O)O)CCN(CC(=O)O)CCN2CC(=O)O)cc1 ; *CN1CCN(CC(=O)O)CCN(CC(=O)O)CCN(CC(=O)O)CC1]

In certain embodiments, each chelating agent can be optionally bound to an isotope (or metal) suitable for radio-imaging, radiotherapy, or MR imaging.

### Conjugates Compounds

In some embodiments, the conjugate (e.g., compound) has the structure:

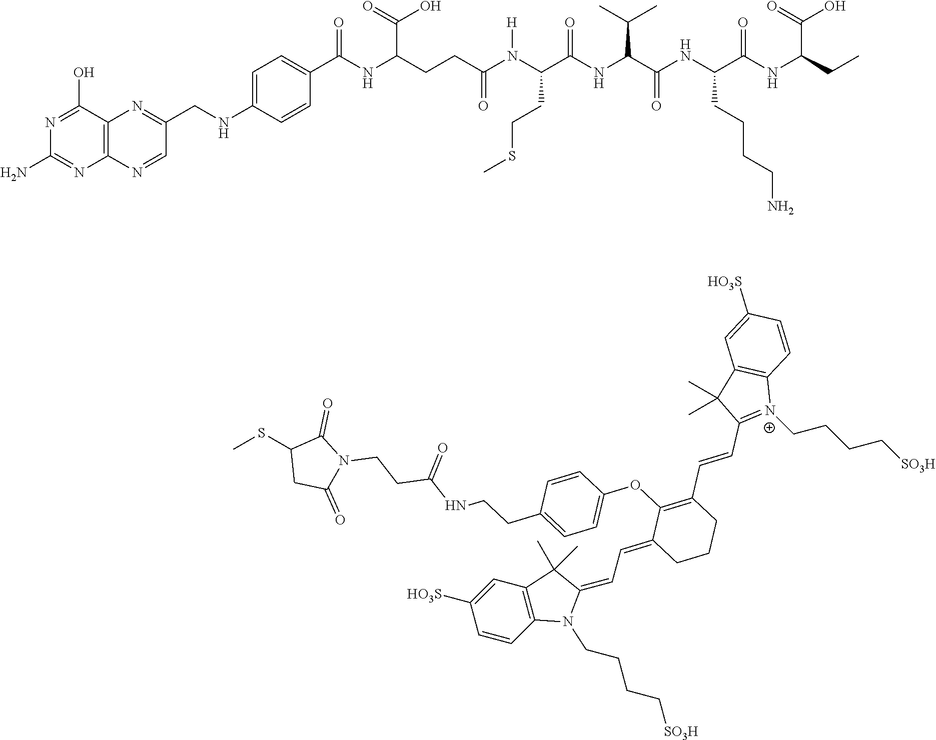

### (BBM1 (MVKC).

In some embodiments, the conjugate has the structure:

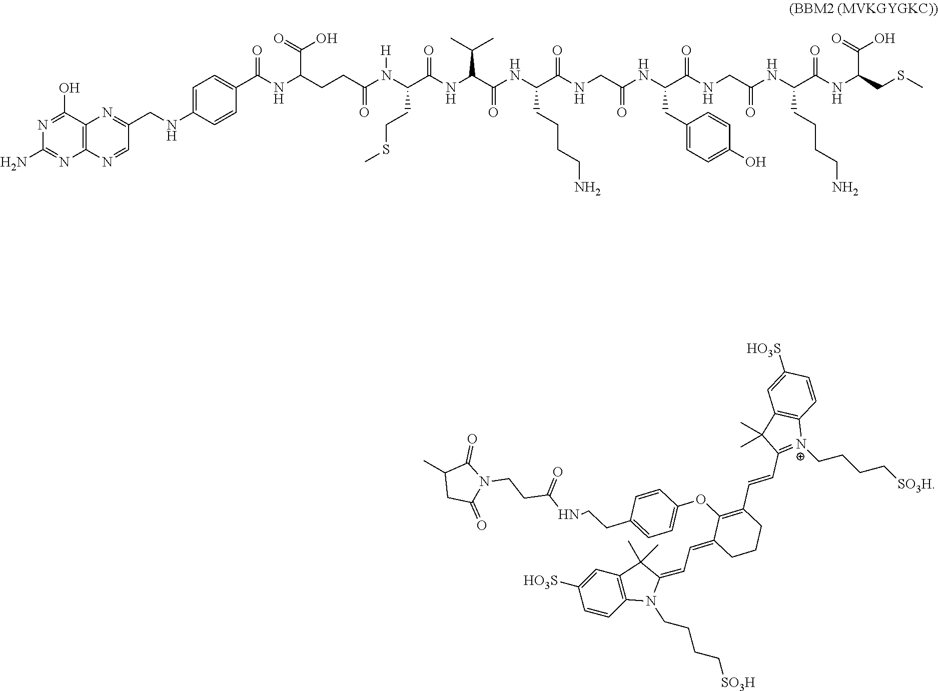

In some embodiments, the conjugate can have the structure:

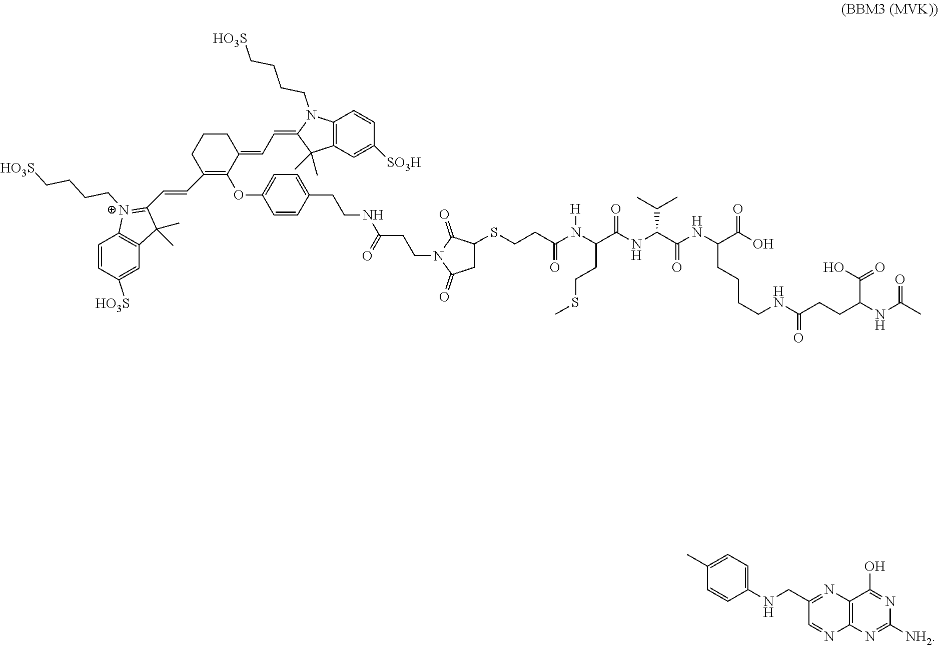

The conjugate can have the structure:

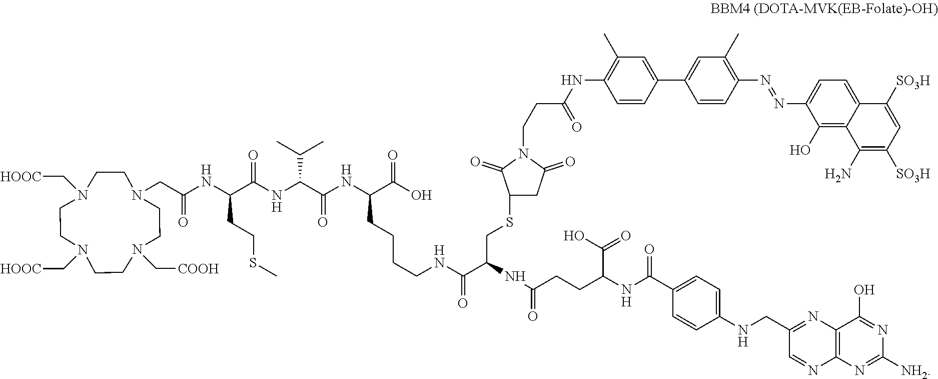 [SMILES: CSCC[C@@H](NC(=O)CN1CCN(CC(=O)O)CCN(CC(=O)O)CCN(CC(=O)O)CC1)C(=O)N[C@@H](C(=O)N[C@H](CCCCNC(=O)[C@@H](CSC1CC(=O)N(CCC(=O)Nc2ccc(-c3ccc(/N=N/c4ccc5c(S(=O)(=O)O)cc(S(=O)(=O)O)c(N)c5c4O)c(C)c3)cc2C)C1=O)NC(=O)CCC(NC(=O)c1ccc(NCc2cnc3nc(N)nc(O)c3n2)cc1)C(=O)O)C(=O)O)C(C)C]

The conjugate can have the structure:

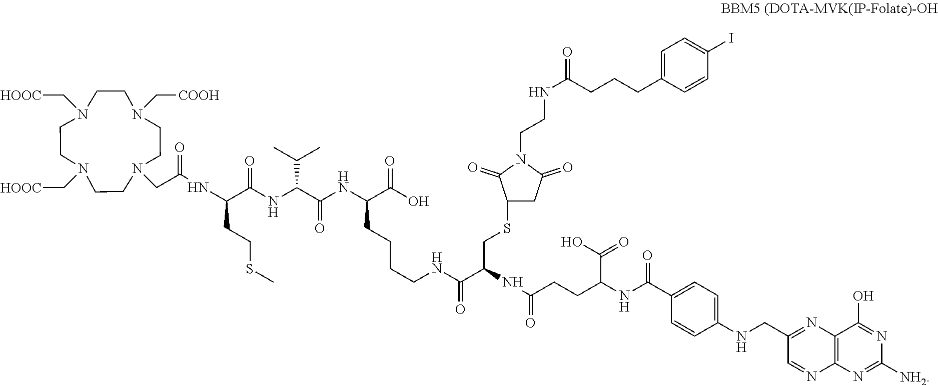 [SMILES: CSCC[C@@H](NC(=O)CN1CCN(CC(=O)O)CCN(CC(=O)O)CCN(CC(=O)O)CC1)C(=O)N[C@@H](C(=O)N[C@H](CCCCNC(=O)[C@@H](CSC1CC(=O)N(CCNC(=O)CCCc2ccc(I)cc2)C1=O)NC(=O)CCC(NC(=O)c1ccc(NCc2cnc3nc(N)nc(O)c3n2)cc1)C(=O)O)C(=O)O)C(C)C]

The conjugate can have the structure:

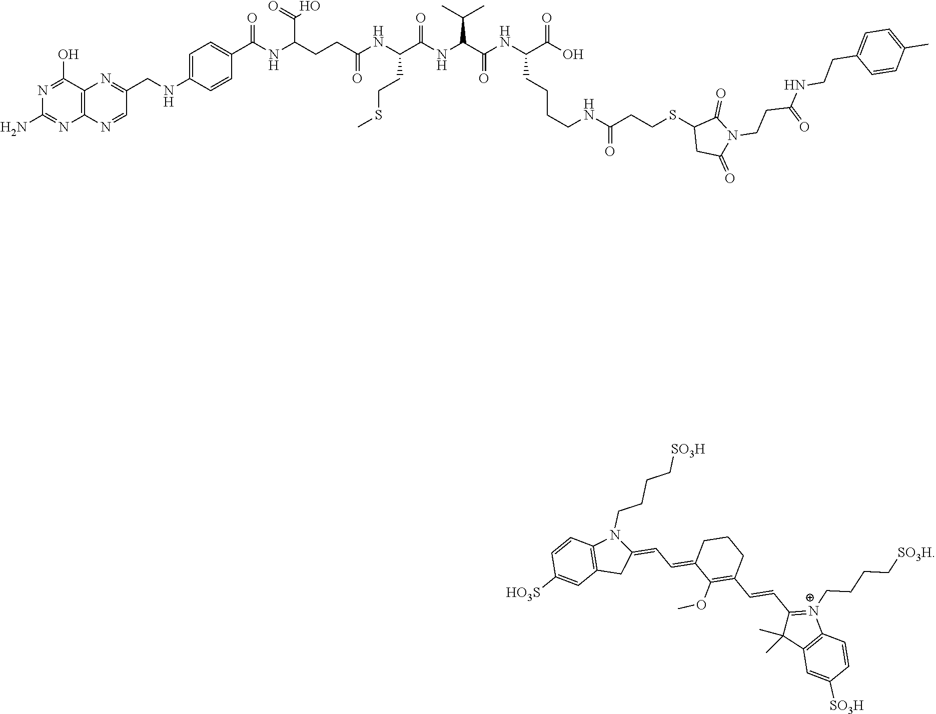

The conjugate can have the structure:

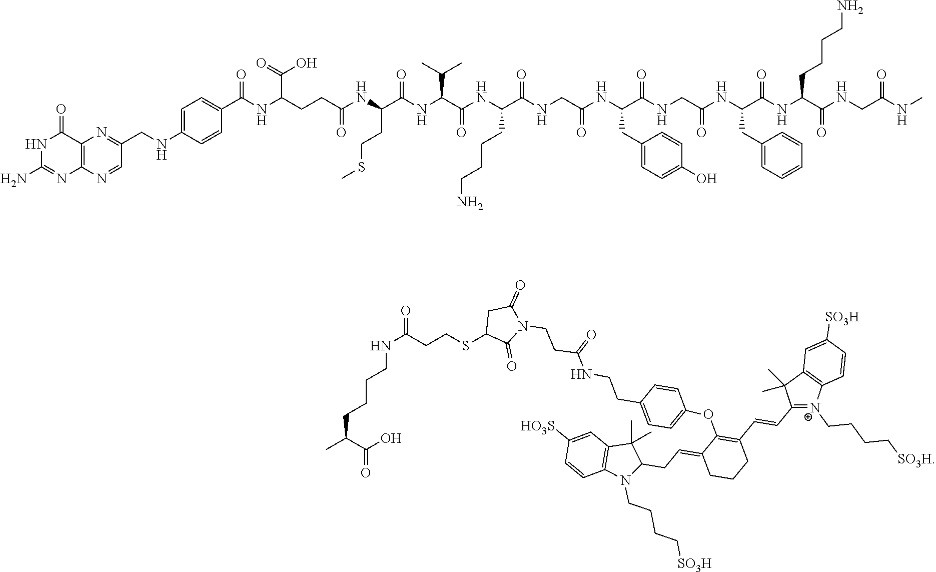

The conjugate can have the structure:

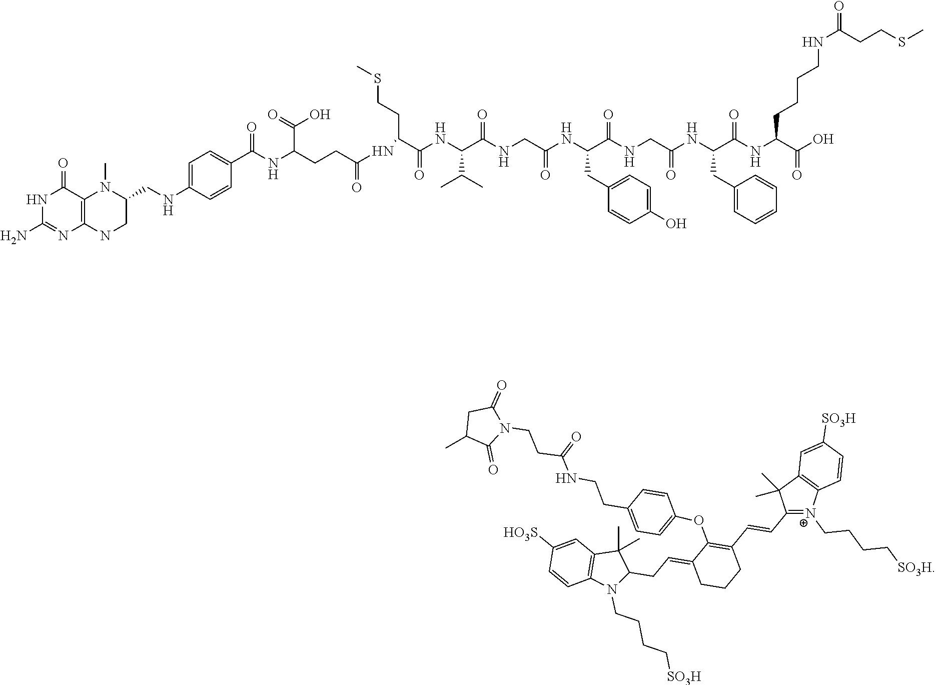

The conjugate can have the structure:

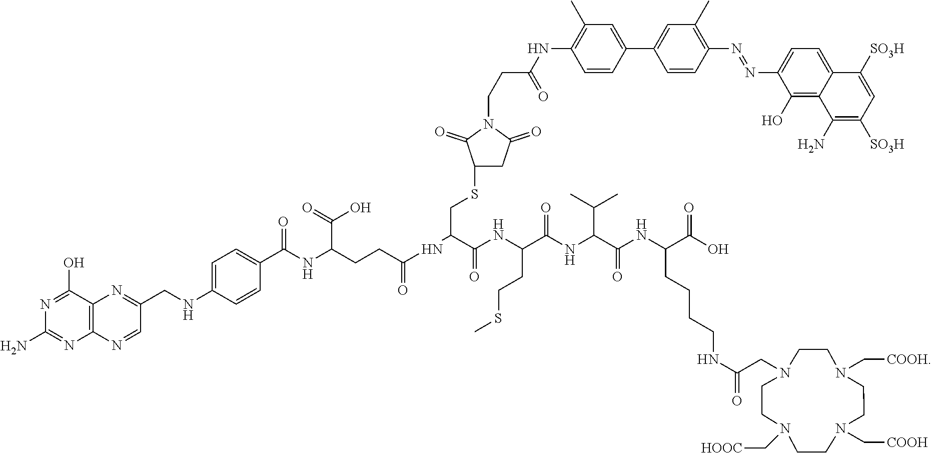 [SMILES: CSCCC(NC(=O)C(CSC1CC(=O)N(CCC(=O)Nc2ccc(-c3ccc(/N=N/c4ccc5c(S(=O)(=O)O)cc(S(=O)(=O)O)c(N)c5c4O)c(C)c3)cc2C)C1=O)NC(=O)CCC(NC(=O)c1ccc(NCc2cnc3nc(N)nc(O)c3n2)cc1)C(=O)O)C(=O)NC(C(=O)NC(CCCCNC(=O)CN1CCN(CC(=O)O)CCN(CC(=O)O)CCN(CC(=O)O)CC1)C(=O)O)C(C)C]

The conjugate can have the structure:

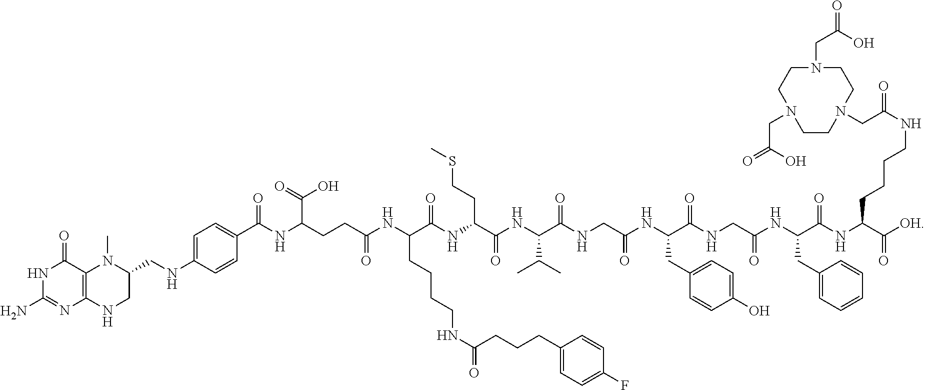 [SMILES: CSCC[C@@H](NC(=O)C(CCCCNC(=O)CCCc1ccc(F)cc1)NC(=O)CCC(NC(=O)c1ccc(NC[C@@H]2CNc3nc(N)[nH]c(=O)c3N2C)cc1)C(=O)O)C(=O)N[C@H](C(=O)NCC(=O)N[C@@H](Cc1ccc(O)cc1)C(=O)NCC(=O)N[C@@H](Cc1ccccc1)C(=O)N[C@@H](CCCCNC(=O)CN1CCN(CC(=O)O)CCN(CC(=O)O)CC1)C(=O)O)C(C)C]

The conjugate can have the structure:

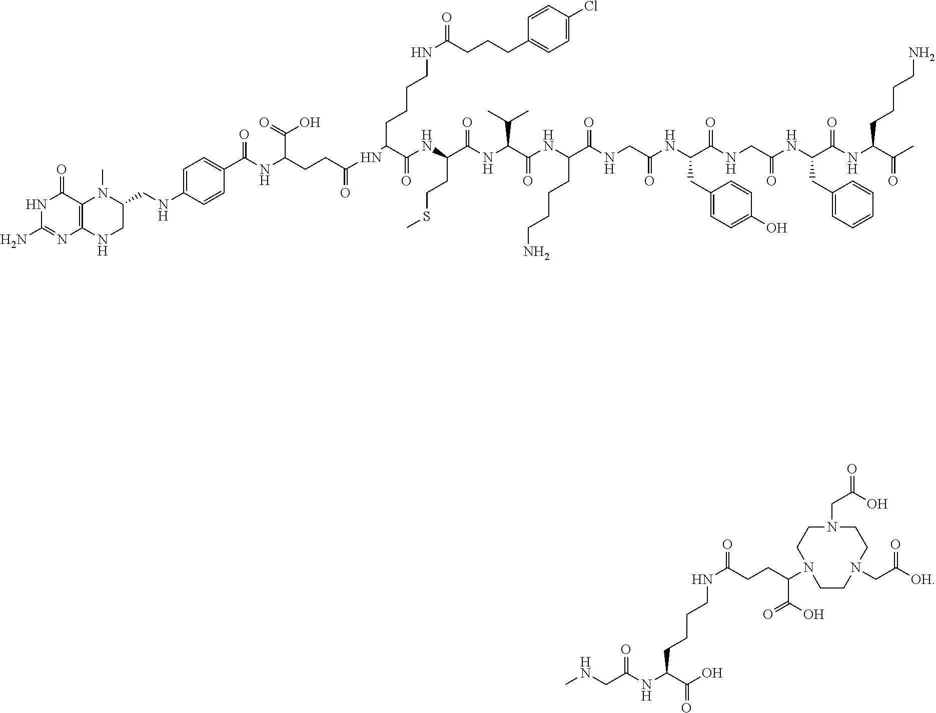 [SMILES: CSCC[C@@H](NC(=O)C(CCCCNC(=O)CCCc1ccc(Cl)cc1)NC(=O)CCC(NC(=O)c1ccc(NC[C@@H]2CNc3nc(N)[nH]c(=O)c3N2C)cc1)C(=O)O)C(=O)N[C@H](C(=O)NC(CCCCN)C(=O)NCC(=O)N[C@@H](Cc1ccc(O)cc1)C(=O)NCC(=O)N[C@@H](Cc1ccccc1)C(=O)N[C@@H](CCCCN)C(C)=O)C(C)C ; CNCC(=O)N[C@@H](CCCCNC(=O)CCC(C(=O)O)N1CCN(CC(=O)O)CCN(CC(=O)O)CC1)C(=O)O]

The conjugates can contain one or more chiral centers or may otherwise exist as multiple stereoisomers, such as enantiomers, diastereomers, and enantiomerically or diastereomerically enriched mixtures. Unless stated otherwise, it is intended that all stereoisomeric forms of the compounds are contemplated. When the conjugates contain alkene double bonds, and unless specified otherwise, it is intended that this includes both E and Z geometric isomers (e.g., cis or trans). Likewise, all possible isomers, as well as their racemic and optically pure forms, and all tautomeric forms are also intended to be included.

The conjugates can exist as geometric isomers. The term “geometric isomer” refers to E or Z geometric isomers (e.g., cis or trans) of an alkene double bond. One of ordinary skill in the art will further appreciate that the compounds can be “deuterated,” meaning one or more hydrogen atoms can be replaced with deuterium.

The conjugates can exist in un-solvated forms as well as solvated forms, including hydrated forms. In general, the solvated forms are equivalent to un-solvated forms. The conjugates can exist in multiple crystalline or amorphous forms. In general, all physical forms are equivalent for the uses contemplated. The formulae include pharmaceutically acceptable salts (e.g., acid addition and base salts), hydrates, and/or solvates.

### Compositions, Routes of Administration, and Dosing

In certain embodiments, the pharmaceutical composition comprises a plurality of conjugates and a pharmaceutically acceptable carrier. The term “pharmaceutically acceptable carrier” means one or more compatible solid or liquid fillers, diluents or encapsulating substances which are suitable for administration to a human or other vertebrate animal. The term “carrier” denotes an organic or inorganic ingredient, natural or synthetic, with which the active ingredient is combined to facilitate the application. The carrier can be an excipient. The choice of carrier can depend on factors such as the particular mode of administration, the effect of the carrier on solubility and stability, and the nature of the dosage form. Pharmaceutical compositions suitable for the delivery of conjugates as described herein and methods for their preparation may be found, for example, in Remington: The Science & Practice of Pharmacy, 21st edition (Lippincott Williams & Wilkins, 2005).

The components of the compositions also can be commingled with the compounds, and with each other, in a manner such that there is no interaction which would substantially impair the desired pharmaceutical efficiency.

Compositions can be prepared by combining one or more conjugates with a pharmaceutically acceptable carrier and, optionally, one or more additional ingredients. The formulations can be administered in pharmaceutically acceptable solutions, which can routinely contain pharmaceutically acceptable concentrations of salt, buffering agents, preservatives, compatible carriers, adjuvants, and optionally other therapeutic ingredients.

The conjugates and optionally one or more other therapeutic agents can be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable acid addition salts are formed from acids which form non-toxic salts. Illustrative examples include, but are not limited to, acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotionate, nitrate, orotate, oxalate, palmitate, pamoate, phosphate/hydrogen phosphate/dihydrogen phosphate, saccharate, stearate, succinate, tartrate, tosylate, and trifluoroacetate salts.

Suitable base salts of the conjugates described herein are formed from bases which form non-toxic salts. Illustrative examples include, but are not limited to, arginine, benzathine, calcium, choline, diethylamine, diolamine, glycine, lysine, magnesium, meglumine, olamine, potassium, sodium tromethamine and zinc salts. Hemisalts of acids and bases, such as hemisulphate and hemicalcium salts, may also be formed.

Compositions and/or dosage forms for administration can be prepared from a conjugate with a purity of at least approximately 90%, approximately 95%, approximately 96%, approximately 97%, approximately 98%, approximately 99%, or approximately 99.5%. Compositions and/or dosage forms for administration can be prepared from a conjugate with a purity of at least 90%, at least 95%, at least 96%, at least 97%, at least 98%, at least 99%, or at least 99.5%.

A pharmaceutically acceptable carrier can include any and all solvents, dispersion media, coatings, antibacterial and antifungal agents, isotonic and absorption delaying agents, and the like, and combinations thereof, that are physiologically compatible. The carrier can be suitable for parenteral administration. Pharmaceutically acceptable carriers include sterile aqueous solutions or dispersions and sterile powders for the extemporaneous preparation of sterile injectable solutions or dispersions. Examples of such carriers (or excipients) include, but are not limited to, calcium carbonate, calcium phosphate, various sugars, starches, cellulose derivatives, gelatin, and polymers such as polyethylene glycols. One or more other active agents also can be incorporated into a composition.

The composition can be formulated as a liquid, e.g., a suspension or a solution. A liquid formulation can comprise water, ethanol, polyethylene glycol, propylene glycol, methylcellulose, or a suitable oil, and one or more emulsifying agents and/or suspending agents. A liquid formulation can be prepared by the reconstitution of a solid.

Pharmaceutical formulations (e.g., for parenteral administration) include aqueous solutions of the active compounds in water-soluble form. Additionally, suspensions of the active conjugates/compounds can be prepared as appropriate oily injection suspensions. An aqueous suspension can contain a conjugate, alone or in further combination with one or more other active agents, in admixture with an appropriate excipient. Excipients include suspending agents, such as sodium carboxymethylcellulose, methylcellulose, hydroxypropylmethylcellulose, sodium alginate, polyvinylpyrrolidone, gum tragacanth and gum acacia; dispersing or wetting agents, such as a naturally-occurring phosphatide, e.g., lecithin; a condensation product of an alkylene oxide with a fatty acid, e.g., polyoxyethylene stearate; a condensation product of ethylene oxide with a long-chain aliphatic alcohol, e.g., heptadecaethyleneoxcycetanol; a condensation product of ethylene oxide with a partial ester derived from fatty acids and a hexitol, such as polyoxyethylene sorbitol monooleate; or a condensation product of ethylene oxide with a partial ester derived from fatty acids and hexitol anhydrides, e.g., polyoxyethylene sorbitan monooleate. The aqueous suspension also can contain one or more preservatives, e.g., ascorbic acid or ethyl, n-propyl, or p-hydroxybenzoate, and one or more coloring agents. In certain embodiments, an aqueous suspension can further comprise suitable lipophilic solvents or vehicles including fatty oils such as sesame oil, or synthetic fatty acid esters, such as ethyl oleate or triglycerides, or liposomes. Optionally, the suspension can also contain suitable stabilizers or agents that increase the solubility of the conjugates to allow for the preparation of highly concentrated solutions.

Alternatively, the compositions can be in powder form for constitution with a suitable vehicle, e.g., sterile pyrogen-free water, before use. Dispersible powders and granules suitable for preparation of an aqueous suspension by the addition of water can provide the active ingredient in admixture with a suspending agent, a dispersing or wetting agent, and one or more preservatives. Additional excipients, for example, coloring agents, also can be present.

Suitable emulsifying agents include naturally occurring gums, e.g., gum acacia or gum tragacanth; naturally occurring phosphatides, e.g., soybean lecithin; and esters, including partial esters derived from fatty acids and hexitol anhydrides, e.g., sorbitan mono-oleate, and condensation products of partial esters with ethylene oxide, e.g., polyoxyethylene sorbitan monooleate. Isotonic agents, e.g., sugars, polyalcohols, such as mannitol, sorbitol, or sodium chloride, can be included in the composition. Prolonged absorption of injectable compositions can be achieved by including in the composition one or more agents to delay absorption, e.g., monostearate salts and gelatin.

For use in therapy or treatment, an effective amount of the conjugate or composition can be administered to a subject by any mode that delivers the compound to the desired surface. Administering a composition can be accomplished by any means known to the skilled artisan. Routes of administration include, but are not limited to, intravenous, intramuscular, intraperitoneal, intravesical (urinary bladder), oral, subcutaneous, direct injection (e.g., into a tumor or abscess), mucosal (e.g., topical to eye), inhalation, and topical.

For oral administration, the conjugates can be formulated readily by combining the conjugates(s) with pharmaceutically acceptable carriers well-known in the art. Such carriers can enable the conjugates to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl pyrrolidone (PVP). If desired, disintegrating agents can be added, such as cross-linked PVP, agar, or alginic acid or a salt thereof such as sodium alginate. Optionally the oral formulations can also be formulated in saline or buffers, e.g., EDTA for neutralizing internal acid conditions, or can be administered without any carriers.

Also contemplated are oral dosage forms of the compounds. The conjugates can be chemically modified so that oral delivery of the derivative is efficacious. Generally, the chemical modification contemplated is the attachment of at least one moiety to the compound itself, where the moiety permits (a) inhibition of acid hydrolysis; and (b) uptake into the blood stream from the stomach or intestine. Additionally or alternatively, the conjugates can be modified to increase their overall stability and circulation time in the body. Examples of the moieties, which can be used to increase stability and/or circulation time, include polyethylene glycol, copolymers of ethylene glycol and propylene glycol, carboxymethyl cellulose, dextran, polyvinyl alcohol, PVP and polyproline. See, e.g., Abuchowski and Davis, “Soluble Polymer-Enzyme Adducts,” In: Enzymes as Drugs, Hocenberg and Roberts, eds., Wiley-Interscience, New York, N.Y., pp. 367-383 (1981); Newmark et al., J Appl Biochem 4:185-189 (1982). Other polymers that can be used are poly-1,3-dioxolane and poly-1,3,6-tioxocane. For pharmaceutical usage, as indicated above, polyethylene glycol moieties are suitable.

Colorants and/or flavoring agents can be included. For example, the compound can be formulated (such as by liposome or microsphere encapsulation) and then further contained within an edible product, such as a refrigerated beverage containing colorants and flavoring agents.

Illustrative formats for oral administration include, but are not limited to, tablets, capsules, elixirs, syrups, and the like.

In certain embodiments, a conjugate can be administered directly into the blood stream, into muscle, or into an internal organ. Suitable routes for such parenteral administration include intravenous, intraarterial, intraperitoneal, intrathecal, epidural, intracerebroventricular, intraurethral, intrasternal, intracranial, intratumoral, intramuscular, intranasal, and subcutaneous. Suitable means for parenteral administration include needle (including microneedle) injectors, needle-free injectors, and infusion techniques. In embodiments where it is desirable to deliver the conjugates and/or compositions systemically, the conjugate(s) and/or composition can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

Parenteral formulations are typically aqueous solutions that can contain carriers or excipients, such as salts, carbohydrates, and buffering agents (preferably at a pH of 3-9), but, for some applications, they may be more suitably formulated as a sterile non-aqueous solution or as a dried form to be used in conjunction with a suitable vehicle, such as sterile, pyrogen-free water.

A liquid formulation can be adapted for parenteral administration of a conjugate. The preparation of parenteral formulations under sterile conditions, for example, by lyophilization under sterile conditions, can readily be accomplished using standard pharmaceutical techniques well-known to those skilled in the art. The solubility of a conjugate can be increased by the use of appropriate formulation techniques, such as the incorporation of solubility-enhancing agents.

Formulations for parenteral administration can be formulated for immediate and/or modified release. A conjugate can be administered in a time-release formulation, for example in a composition which includes a slow-release polymer. The conjugate can be prepared with a carrier that will protect it against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PGLA). Methods for the preparation of such formulations are generally known to those skilled in the art.

Sterile injectable solutions can be prepared by incorporating the conjugate(s), alone or in further combination with one or more other active agents, in the required amount in an appropriate solvent with one or a combination of ingredients described above, as required, followed by filtered sterilization. Typically, dispersions are prepared by incorporating the conjugate into a sterile vehicle, which contains a dispersion medium and any additional ingredients of those described above. In the case of sterile powders for the preparation of sterile injectable solutions, the preferred methods of preparation are vacuum-drying and freeze-drying, which yield a powder of the active ingredients plus any additional desired ingredient from a previously sterile-filtered solution thereof, or the ingredients can be sterile-filtered together.

The composition can be formulated as a solution, microemulsion, liposome, or other ordered structure suitable to high drug concentration. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating, such as lecithin, by the maintenance of the required particle size in the case of dispersion, and by the use of surfactants.

A conjugate, or a composition comprising a conjugate, can be continuously administered, where appropriate.

### Kits

A kit is still further provided. If more than one conjugate is to be administered or if a conjugate is to be administered, simultaneously or sequentially (in either order) with one or more other active agents, the conjugates and active agents (or compositions comprising them) can be combined in a kit. In at least one embodiment, the kit comprises at least one dosage unit of the conjugate or a composition comprising the conjugate. In at least one embodiment, the kit comprises at least one dosage unit of a second active agent or a composition comprising the second active agent. “Dosage unit” means the composition or conjugate administered in one administration by one delivery operation. For example, in an embodiment where the composition is formulated for transmucosal administration by nasal delivery, a dosage unit is the volume of the composition administered or amount of active agent administered by one delivery operation.

The conjugates and active agents can be in the same or separate containers, e.g., vials, divided bottle, divided foil packet, and the like, in solid or liquid form. The kit also can contain instructions for use. The instructions can be printed on paper, supplied in electronic-readable media, or accessed on the Internet, such as via email, text, social media, a website, and the like. In certain embodiments, the kit further comprises a means for administration of the conjugate and/or active agents during a treatment. Such a means for administration can, for example, include a syringe, a tourniquet, a stent, a cannula, and/or a trocar

### Methods of Imaging and or Treatment

Still further provided is a method of imaging, treating, or imaging and treating a tumor (e.g., cancer) in a subject by targeting radioactivity, alone or in further combination with an optical imaging agent, to cells of the tumor, macrophages associated with the tumor (tumor-associated macrophages (TAMs)), or both. In certain embodiments, the method comprises administering to the subject an effective amount of a conjugate or a composition comprising the conjugate and a pharmaceutically acceptable carrier. The method can further comprise imaging the tumor. The imaging can be by optical imaging, PET, or SPECT.

In certain embodiments, the method of imaging, treating, or imaging and treating a tumor (e.g., cancer) in a subject comprises contacting a tumor cell (e.g., of a cancer patient) with a compound (e.g., a conjugate) of any formula provided herein.

Thus, a method for imaging cancer (e.g., a tumor) in a subject with the cancer is provided. In certain embodiments, the method comprises administering, to the subject, an effective amount of a conjugate (e.g., as part of a pharmaceutical composition or otherwise). In certain embodiments, the method further comprises imaging the subject. In certain embodiments, the method further comprises generating an image of the cancer (e.g., tumor) in the subject (e.g., after or concurrently with administration of the conjugate).

Also provided are methods for optical imaging. In certain embodiments, a method for optical imaging a subject comprises administering, to the subject, an effective amount of any conjugate (e.g., as part of a pharmaceutical composition or otherwise). The methods can be for fluorescence-guided surgery for example. The methods can be for radio-imaging. The methods can be for MRI.

The methods can be used in combination with one or more additional therapies and/or active agents. Such additional therapies include, without limitation, an immunotherapy, the administration of a DNA damage response pathway inhibitor, chemotherapy, and/or a surgery.

“Effective amount” refers to an amount of a conjugate, or composition comprising the same, that elicits the desired biological or medicinal response in a subject (i.e., a tissue, organ, or organism, such as a vertebrate, e.g., mammal, such as a human) that is being sought by a researcher, veterinarian, medical doctor, or other clinician, which includes, but is not limited to, imaging and/or alleviation of the signs and/or symptoms of the disease or disorder being treated. In one aspect, the effective amount is an amount of an active agent which may treat or alleviate the signs and/or symptoms of the disease at a reasonable benefit/risk ratio applicable to any medical treatment. An effective amount of a prodrug is an amount of an inactive prodrug which, when converted through normal metabolic processes, produces an amount of active drug that elicits the desired biological or medicinal response in a subject that is being sought. An “effective amount” with respect to use in treatment, refers to an amount of the compound in a preparation which, when administered as part of a desired dosage regimen (e.g., to a mammal, such as a human) alleviates a symptom, ameliorates a condition, or slows the onset of disease according to clinically acceptable standards for the disorder or condition to be treated or the cosmetic purpose, e.g., at a reasonable benefit/risk ratio applicable to any medical treatment.

Combined with the teachings provided herein, by choosing among the various active agents and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject.

Depending upon the type of cancer, the route of administration and/or whether the conjugates are administered locally or systemically, a wide range of permissible dosages are contemplated herein, including doses falling in the range from about 1 μg/kg to about 1 g/kg. The unitary daily dosage can vary significantly depending on the patient condition, the cancer being treated, the route of administration, tissue distribution, and the possibility of co-usage of other therapeutic treatments, such as radiation therapy or additional drugs in combination therapies. The effective amount to be administered to a patient is based on body surface area, mass, and physician assessment of patient condition. Therapeutically effective doses (also referred to herein as “therapeutically effective amounts” or “effective amounts”) can range, for example, from approximately 0.5 to 20.0 mg/m².

For any conjugate, effective amount can be initially determined from animal models. An effective dose can also be determined from human data for compounds which have been tested in humans and for conjugates/compounds that are known to exhibit similar pharmacological activities, such as other related active agents. Higher doses may be required for parenteral administration. The effective amount for any particular application can vary depending on such factors as the disease or condition being treated, the particular conjugate(s) being administered, the size of the subject, or the severity of the disease or condition. One of ordinary skill in the art can empirically determine the effective amount of a particular compound and/or other therapeutic agent without necessitating undue experimentation. A maximum dose can be used, that is, the highest safe dose according to some medical judgment. Multiple doses per day can be used to achieve appropriate systemic levels of compounds. Appropriate systemic levels can be determined by, for example, measurement of the patient's peak or sustained plasma level of the drug. “Dose” and “dosage” are used interchangeably herein.

Adjusting the dose to achieve maximal efficacy based on the methods described and other methods well-known in the art is well within the capabilities of the ordinarily skilled artisan. Dosage can be adjusted appropriately to achieve desired drug levels, local or systemic, depending upon the mode of administration. For example, the dose for intravenous administration can vary from one order to several orders of magnitude lower per day. If the response in a subject is insufficient at such doses, even higher doses (or effective higher doses by a different, more localized delivery route) can be employed to the extent that subject tolerance permits. Multiple doses per day are contemplated to achieve appropriate systemic levels of the compound.

Any effective regimen for administering the conjugates can be used. The dosages can be single or divided and can be administered according to a wide variety of protocols, including q.d., b.i.d., t.i.d., or even every other day, biweekly (b.i.w.), once a week, once a month, once a quarter, and the like. In each of these cases it is understood that the effective amounts described herein correspond to the instance of administration, or alternatively to the total daily, weekly, month, or quarterly dose, as determined by the dosing protocol.

For example, conjugates can be administered as single doses, or the doses can be divided and administered as a multiple-dose daily regimen. Further, a staggered regimen, for example, one to five days per week can be used as an alternative to daily treatment. Such intermittent or staggered daily regimen is considered equivalent to daily treatment. The patient can be treated with multiple injections of a conjugate to treat cancer. The patient can be injected multiple times (e.g., approximately 2-50×) with a conjugate, for example, at 12-72 hours intervals or at 48-72 hours intervals. Additional injections of a conjugate can be administered to the patient at an interval of days or months after the initial injection(s), and the additional injections can prevent the recurrence of the cancer.

Alternatively, individual doses and dosage regimens can be selected, for example, to provide a total dose administered during a month of about 15 mg. A conjugate can be administered in a single daily dose five days per week, in weeks 1, 2, and 3 of each 4-week cycle, with no dose administered in week 4. In yet another alternative example, a conjugate can be administered in a single daily dose administered three days per week, in weeks 1 and 3 of each 4-week cycle, with no dose administered in weeks 2 and 4. In still yet another alternative example, a conjugate can be administered biweekly in weeks 1 and 2 (i.e., on days 1, 4, 8, and 11 of a 3-week cycle). As a further alternative example, a conjugate can be administered once weekly in weeks 1 and 2 (i.e., days 1 and 8 of a 3-week cycle).

A “subject” can be a human patient, a laboratory animal, such as a rodent (e.g., mouse, rat, or hamster), a rabbit, a monkey, a chimpanzee, a domestic animal, such as a dog, a cat, or a rabbit, an agricultural animal, such as a cow, a horse, a pig, a sheep, or a goat, or a wild animal in captivity, such as a bear, a panda, a lion, a tiger, a leopard, an elephant, a zebra, a giraffe, a gorilla, a dolphin, or a whale.

The cancer can be a cancer cell population that is tumorigenic, including benign tumors and malignant tumors, or non-tumorigenic. The cancer can arise spontaneously, by germline or somatic mutation, or the cancer can be chemically, virally, or radiatively induced. Cancers include, but are not limited to, a carcinoma, a sarcoma, a lymphoma, a melanoma, a mesothelioma, a nasopharyngeal carcinoma, a leukemia, an adenocarcinoma, and a myeloma. The cancer can be lung cancer, bone cancer, pancreatic cancer, skin cancer, cancer of the head, cancer of the neck, cutaneous melanoma, intraocular melanoma, uterine cancer, ovarian cancer, endometrial cancer, leiomyosarcoma, rectal cancer, stomach cancer, colon cancer, breast cancer, triple negative breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, non-small cell lung cancer, small cell lung cancer, cancer of the adrenal gland, sarcoma of soft tissue, cancer of the urethra, cancer of the penis, prostate cancer, chronic leukemia, acute leukemia, lymphocytic lymphomas, pleural mesothelioma, cancer of the bladder, Burkitt's lymphoma, cancer of the ureter, cancer of the kidney, renal cell carcinoma, carcinoma of the renal pelvis, neoplasms of the central nervous system (CNS), primary CNS lymphoma, spinal axis tumors, brain stem glioma, pituitary adenoma, cholangiocarcinoma, Hurthle cell thyroid cancer, or adenocarcinoma of the gastroesophageal junction.

### Other Definitions

Unless defined otherwise, all technical and scientific terms used herein have the same meaning as commonly understood by one of skill in the chemical and biological arts. Additionally, as used in this specification and the appended claims, the singular forms “a”, “an” and “the” include plural referents unless the content clearly dictates otherwise. Thus, for example, where a compound/composition is substituted with “an” alkyl or aryl, the compound/composition is optionally substituted with at least one alkyl and/or at least one aryl.

“Oxo” refers to the ═O radical.

“Alkyl” generally refers to a straight or branched hydrocarbon chain radical consisting solely of carbon and hydrogen atoms, such as having from one to fifteen carbon atoms (e.g., C₁-C₁₅ alkyl). Disclosures provided herein of an “alkyl” are intended to include independent recitations of a saturated “alkyl,” unless otherwise stated. An alkyl can comprise one to thirteen carbon atoms (e.g., C₁-C₁₃ alkyl). An alkyl can comprise one to eight carbon atoms (e.g., C₁-C₈ alkyl). An alkyl can comprise one to five carbon atoms (e.g., C₁-C₅ alkyl). An alkyl can comprise one to four carbon atoms (e.g., C₁-C₄ alkyl). An alkyl can comprise one to three carbon atoms (e.g., C₁-C₃ alkyl). An alkyl can comprise one to two carbon atoms (e.g., C₁-C₂ alkyl). An alkyl can comprise one carbon atom (e.g., C₁ alkyl). An alkyl can comprise five to fifteen carbon atoms (e.g., C₅-C₁₅ alkyl). An alkyl can comprise five to eight carbon atoms (e.g., C₅-C₈alkyl). An alkyl can comprise two to five carbon atoms (e.g., C₂-C₅ alkyl). An alkyl can comprise three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond.

“Alkoxy” refers to a radical bonded through an oxygen atom of the formula —O— alkyl, where alkyl is an alkyl chain as defined above.

“Alkylene” or “alkylene chain” generally refers to a straight or branched divalent alkyl group linking the rest of the molecule to a radical group, such as having from one to twelve carbon atoms, for example, methylene, ethylene, propylene, i-propylene, n-butylene, and the like.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene.

“Aralkyl” or “aryl-alkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain.

“About” can allow for a degree of variability in a value or range, for example, within 10%, within 5%, or within 1% of a stated value or of a stated limit of a range.

The term “comprising” (and related terms such as “comprise” or “comprises” or “having” or “including”) is not intended to exclude an embodiment of any compound, composition, method, process, or the like that may “consist of” or “consist essentially of” the described features. The invention illustratively described herein may be suitably practiced in the absence of any element(s) or limitation(s), which is/are not specifically disclosed herein.

The term “heteroalkyl” refers to an alkyl group as defined above in which one or more skeletal carbon atoms of the alkyl are substituted with a heteroatom (with the appropriate number of substituents or valences—for example, —CH₂— can be replaced with —NH— or —O—). For example, each substituted carbon atom is independently substituted with a heteroatom, such as wherein the carbon is substituted with a nitrogen, oxygen, selenium, or other suitable heteroatom. In some instances, each substituted carbon atom is independently substituted for an oxygen, nitrogen (e.g. —NH—, —N(alkyl)-, or —N(aryl)- or having another substituent contemplated herein), or sulfur (e.g. —S—, —S(═O)—, or —S(═O)₂—). A heteroalkyl is attached to the rest of the molecule at a carbon atom of the heteroalkyl. A heteroalkyl is attached to the rest of the molecule at a heteroatom of the heteroalkyl. A heteroalkyl is a C₁-Cis heteroalkyl. A heteroalkyl is a C₁-C₁₂ heteroalkyl. A heteroalkyl is a C₁-C₆ heteroalkyl. A heteroalkyl is a C₁-C₄ heteroalkyl. Heteroalkyl can include alkoxy, alkoxyalkyl, alkylamino, alkylaminoalkyl, aminoalkyl, heterocycloalkyl, heterocycloalkyl, and heterocycloalkylalkyl, as defined herein.

The term “radical” as used herein refers to a fragment of a molecule, wherein that fragment has an open valence for bond formation. A monovalent radical has one open valence such that it can form one bond with another chemical group. Unless otherwise specified, a radical of a molecule is created by removal of one hydrogen atom from that molecule to create a monovalent radical with one open valence at the location where the hydrogen atom was removed. Where appropriate, a radical can be divalent, trivalent, etc., wherein two, three or more hydrogen atoms or other groups have been removed to create a radical which can bond to two, three, or more chemical groups. Where appropriate, a radical open valence may be created by removal of other than a hydrogen atom (e.g., a halogen), or by removal of two or more atoms (e.g., a hydroxyl group), as long as the atoms removed are a small fraction (20% or less of the atom count) of the total atoms in the molecule forming the radical.

“Substantially” can allow for a degree of variability in a value or range, for example, within 90%, within 95%, or within 99% of a stated value or of a stated limit of a range.

While the concepts of the present disclosure are illustrated and described in detail in the figures and descriptions herein, results in the figures and their description are to be considered as exemplary and not restrictive in character; it being understood that only the illustrative embodiments are shown and described and that all changes and modifications that come within the spirit of the disclosure are desired to be protected. Indeed, the numerous specific details provided are set forth to provide a thorough understanding of the present disclosure. Particular examples may be implemented without some or all of these specific details and it is to be understood that this disclosure is not limited to particular biological systems, particular cancers, or particular organs or tissues, which can, of course, vary, but remain applicable in view of the data provided herein.

The entire contents of each and every patent publication, non-patent publication, and reference text cited herein are hereby incorporated by reference, except that in the event of any inconsistent disclosure or definition from the present specification, the disclosure or definition herein shall be deemed to prevail.

Various techniques and mechanisms of the present disclosure will sometimes describe a connection or link between two components. Words such as attached, linked, coupled, connected, and similar terms with their inflectional morphemes are used interchangeably, unless the difference is noted or made otherwise clear from the context. These words and expressions do not necessarily signify direct connections but include connections through mediate components. It should be noted that a connection between two components does not necessarily mean a direct, unimpeded connection, as a variety of other components may reside between the two components of note. Consequently, a connection does not necessarily mean a direct, unimpeded connection unless otherwise noted.

The use of headings and subheadings is solely for ease of reference and is not intended to limit the scope of the disclosure under a given heading or subheading to the subject matter set forth there under. Rather, disclosure under any heading or subheading is applicable to all subject matter herein, unless expressly indicated otherwise or contradicted by context.

### EXAMPLES

The following examples serve to illustrate the present disclosure and are not intended to limit its scope in any way.

### Example 1

### Synthesis of Folate Ligand with Brush Border Membrane (BBM) Linker M-V-K-C

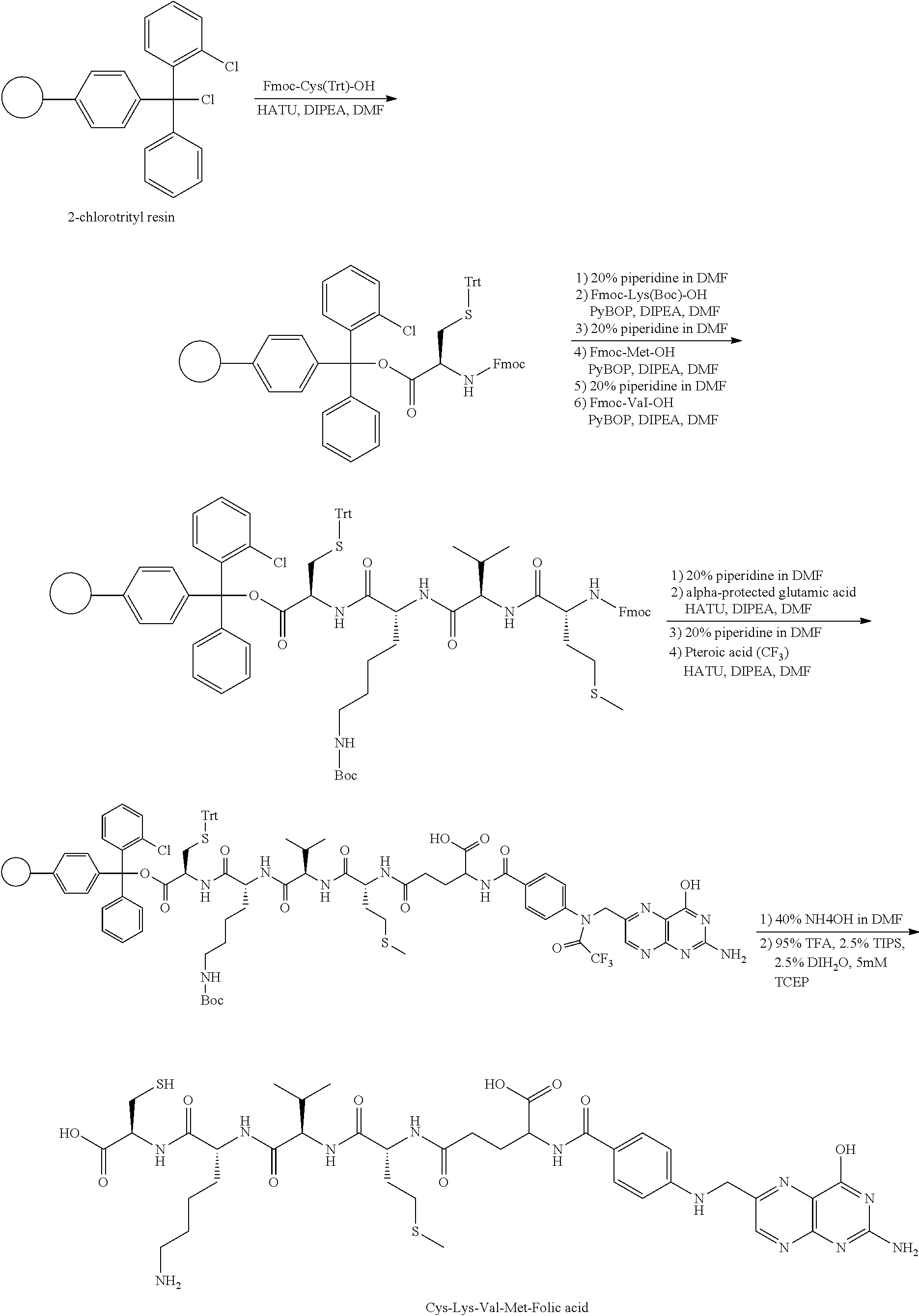

2-chlorotrityl resin was swelled in 10 mL of DCM per gram of resin then filtered. 2.0 equivalents of the first amino acid were dissolved in 10 mL of DCM per gram of resin with 4.0 equivalents of DIPEA, then added to the resin. The mixture was stirred for 2 hours, then capped with the addition of 0.8 mL of MeOH per gram of resin for 30 minutes. The resin was washed twice with DMF, then DCM, then MeOH.

Additional amino acids and peptidomimetic molecules were conjugated to the peptide as follows. First, the Fmoc was deprotected by suspending the resin 3× in 20% piperidine/DMF solution (v/v) for 20 minutes per suspension, and 5 washes of DMF, then DCM, then DMF in between each suspension. Next, 2 equivalents of the appropriate amino were dissolved 20 mL of DMF per gram of resin with 2 equivalents of HATU and 4 equivalents of DIPEA. The solution was incubated for 5 minutes, then added to the resin and bubbled with an inert gas for 2-3 hours.

The coupling step was repeated under the same conditions before proceeding to the next amino acid. 3 equivalents of DOTA-tris(t-Bu ester), HATU, and 6 equivalents of DIPEA were used for coupling. Alloc was deprotected by adding 1 equivalent of Pd(PPh₃)₄ freshly rinsed with MeOH and 20 equivalents of phenylsilane to resin in DCM at a concentration of ˜0.05M under inert gas for 1 hour. The resin was washed three times with DMF, sodium N,N-diethyldithiocarbamate (0.03 M in DMF), and DCM. Pteoric acid protected with COCF₃ was deprotected with 40% NH₄OH/DMF solution (v/v) for 6 hours. Final peptide conjugates were cleaved by incubating a 95% TFA/2.5% TIPS/2.5% H₂O solution with 5 mM TCEP with the resin for 1 hour. Cleavage products were precipitated in ice cold diethyl ether and coupled with maleimide-bearing dyes without further purification.

Final products were purified using RP-HPLC (Agilent Technologies; Santa Clara, CA; C18 10 μm; 19 mm×250 mm).

### Example 2

### Synthesis of 50456-Maleimide and Coupling to BBM Linker of Folate-M-V-K-C-SH (BBM1 (MVKC))

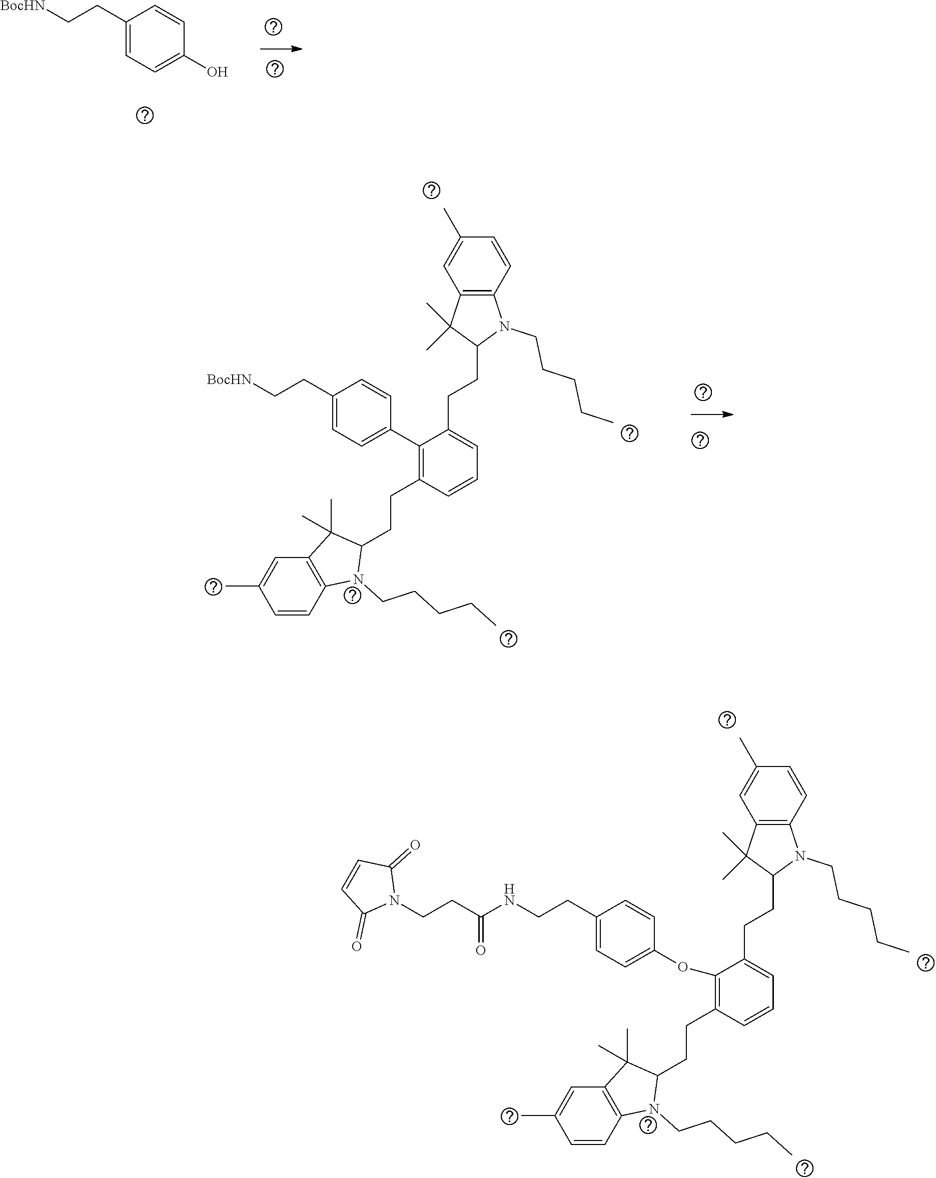 [SMILES: CC(C)(C)OC(=O)NCCc1ccc(O)cc1 ; CCCCCN1c2ccc(C)cc2C(C)(C)C1CCc1cccc(CCC2N(CCCCC)c3ccc(C)cc3C2(C)C)c1-c1ccc(CCNC(=O)OC(C)(C)C)cc1 ; CCCCCN1c2ccc(C)cc2C(C)(C)C1CCc1cccc(CCC2N(CCCCC)c3ccc(C)cc3C2(C)C)c1Oc1ccc(CCNC(=O)CCN2C(=O)C=CC2=O)cc1]

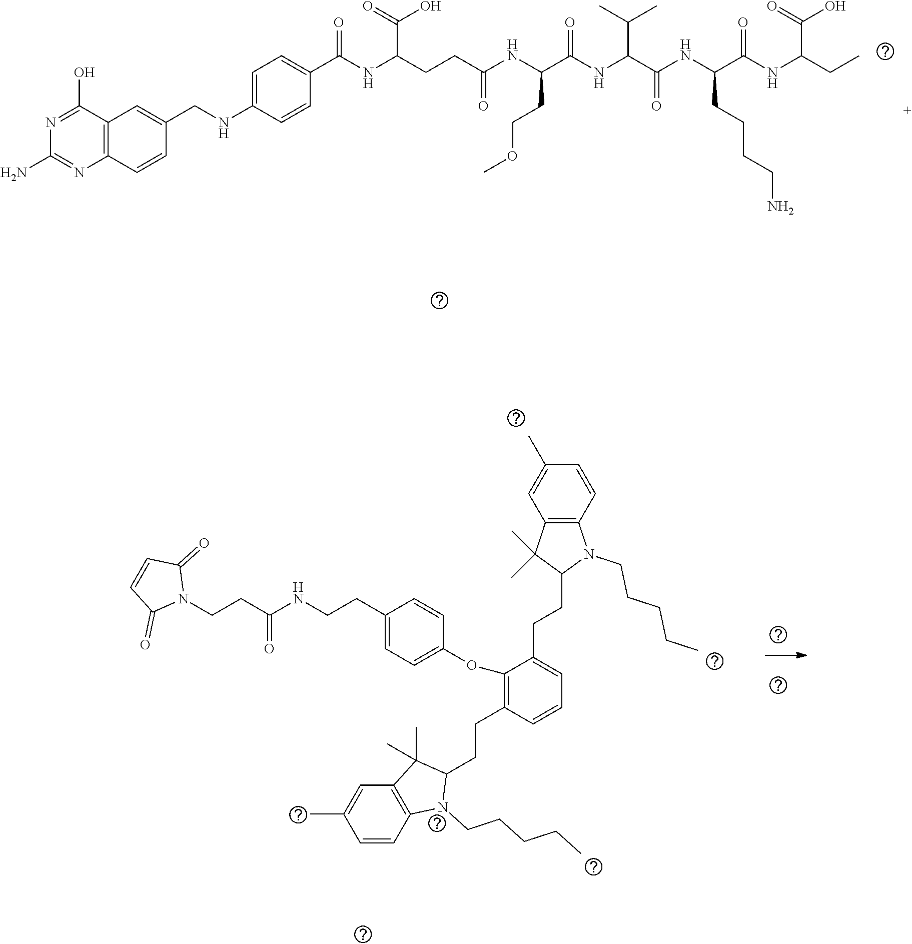 [SMILES: CCC(NC(=O)[C@@H](CCCCN)NC(=O)C(NC(=O)[C@@H](CCOC)NC(=O)CCC(NC(=O)c1ccc(NCc2ccc3nc(N)nc(O)c3c2)cc1)C(=O)O)C(C)C)C(=O)O ; CCCCCN1c2ccc(C)cc2C(C)(C)C1CCc1cccc(CCC2N(CCCCC)c3ccc(C)cc3C2(C)C)c1Oc1ccc(CCNC(=O)CCN2C(=O)C=CC2=O)cc1]

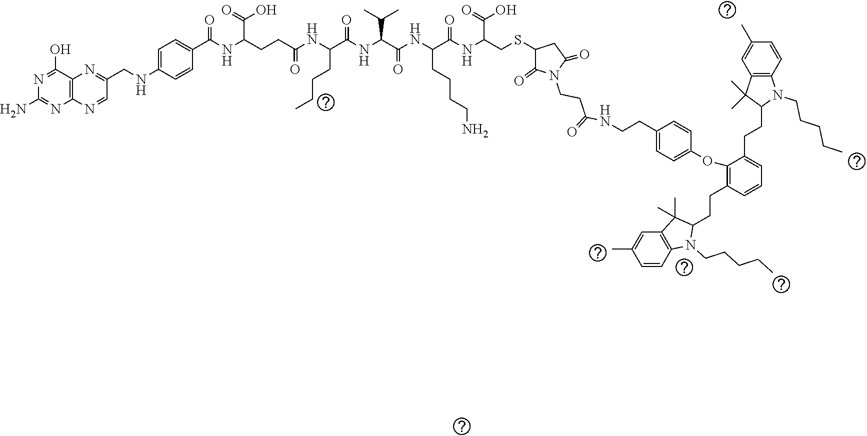 [SMILES: CCCCCN1c2ccc(C)cc2C(C)(C)C1CCc1cccc(CCC2N(CCCCC)c3ccc(C)cc3C2(C)C)c1Oc1ccc(CCNC(=O)CCN2C(=O)CC(SCC(NC(=O)C(CCCCN)NC(=O)[C@@H](NC(=O)C(CCCC)NC(=O)CCC(NC(=O)c3ccc(NCc4cnc5nc(N)nc(O)c5n4)cc3)C(=O)O)C(C)C)C(=O)O)C2=O)cc1]

S0456 maleimide and Evans Blue maleimide (International Publication No. WO/2019/165200) were synthesized following previously published protocols. Final products were purified using RP-HPLC (Agilent Technologies; Santa Clara, CA; C18 10 μm; 19 mm×250 mm).

### Example 3

### Synthesis of BBM3 (MVK)

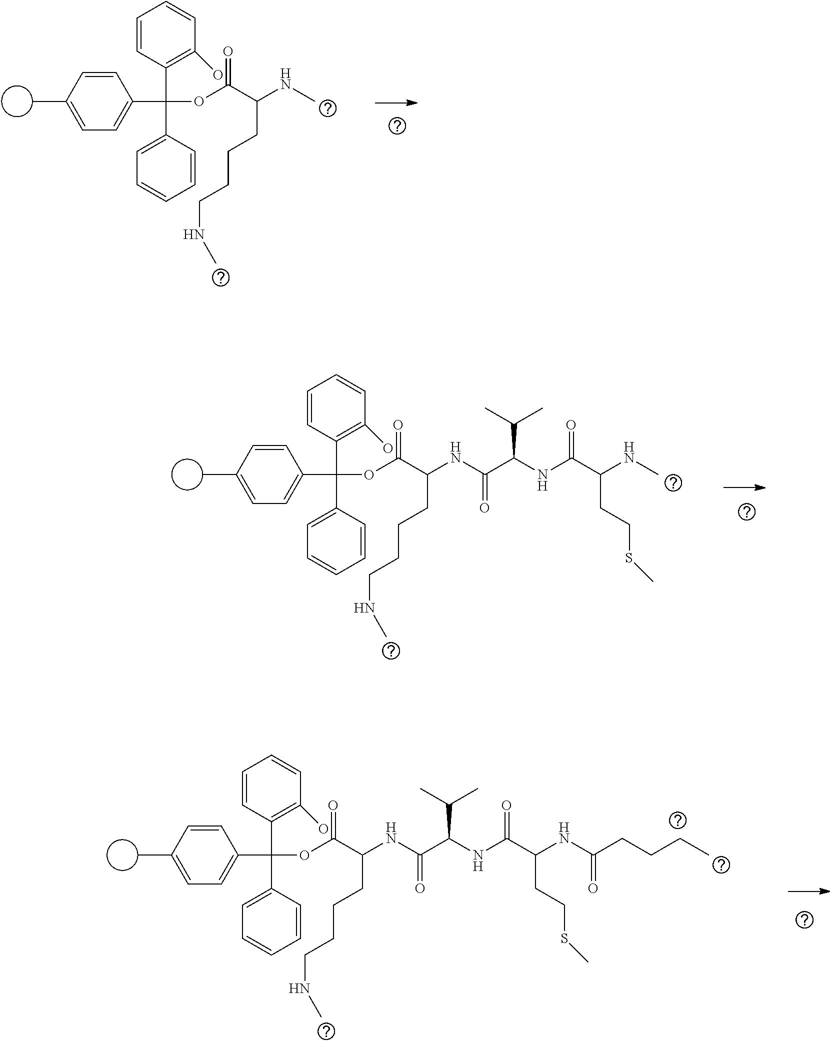

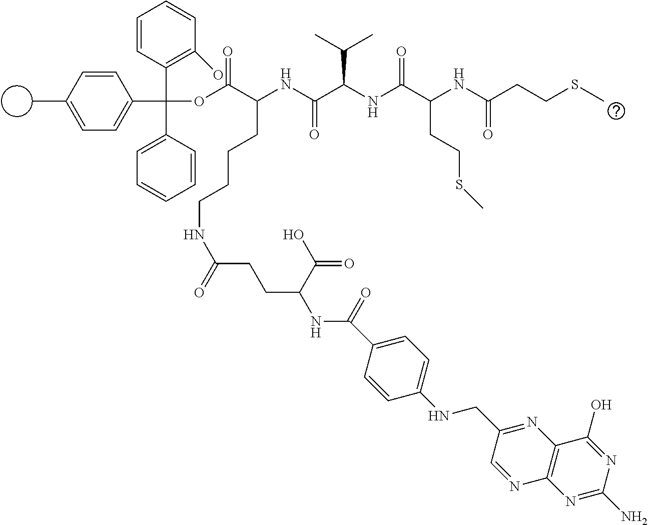 [SMILES: CSCCC(=O)NC(CCSC)C(=O)N[C@@H](C(=O)NC(CCCCNC(=O)CCC(NC(=O)c1ccc(NCc2cnc3nc(N)nc(O)c3n2)cc1)C(=O)O)C(=O)OC(c1ccccc1)(c1ccc(C)cc1)c1ccccc1O)C(C)C]

### Example 4

### Folate Conjugate Binding Curves

KB cells were incubated for 1 hour at 4° C. with increasing concentrations of OTL38, BBM1, BBM2, or BBM3 in staining buffer, washed three times with staining buffer, and then analyzed via flow cytometry.

OTL38 was synthesized as described in Mahalingam et al., Evaluation of Novel Tumor-Targeted Near-Infrared Probe for Fluorescence-Guided Surgery of Cancer, Journal of Medicinal Chemistry 2018 61(21), 9637-9646. BBM2 was synthesized in accordance with the schemes set forth herein and is a peptide with the sequence M-V-K-G-Y-G-K-C.

### Example 5

### Synthesis of Folate BBM Radioligand Conjugate Precursor

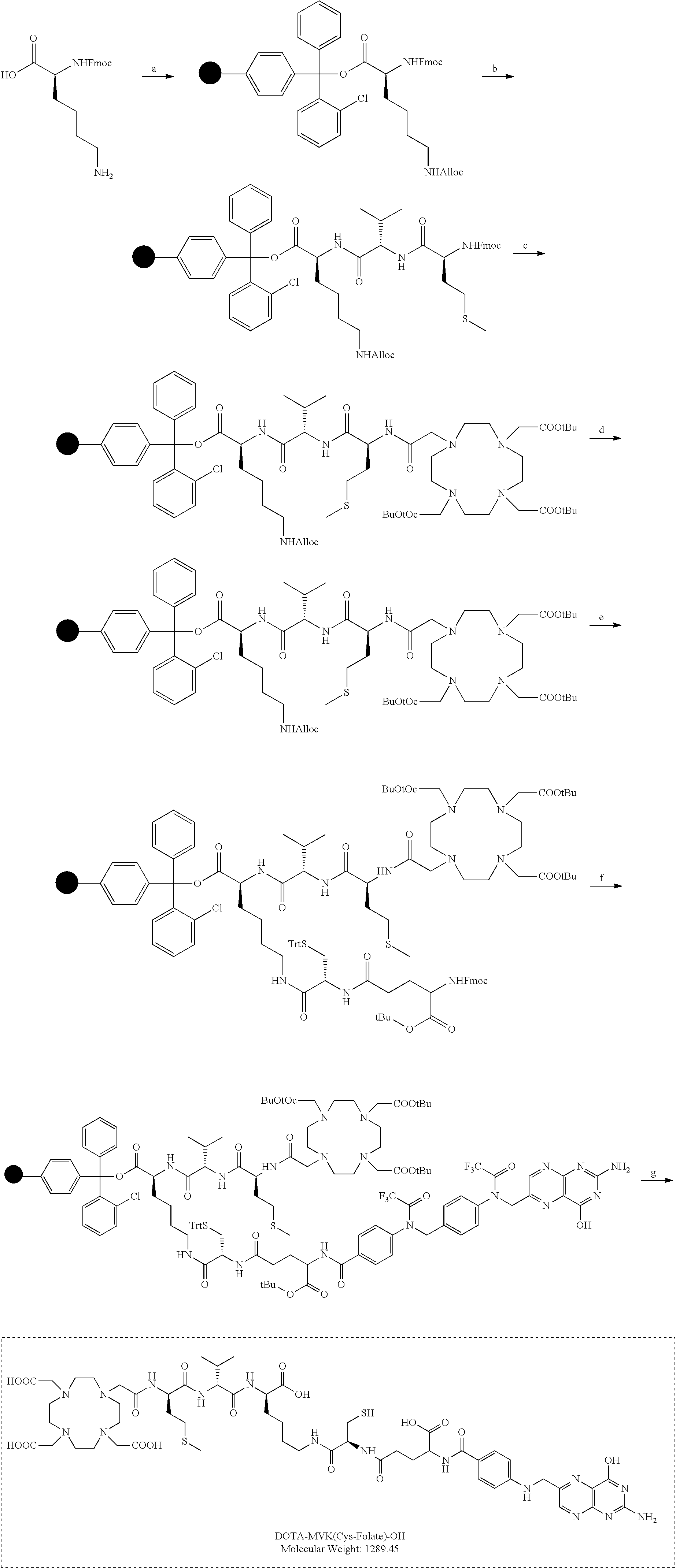

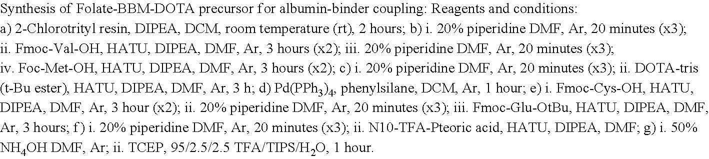

### Example 6

### Synthesis of BBM4

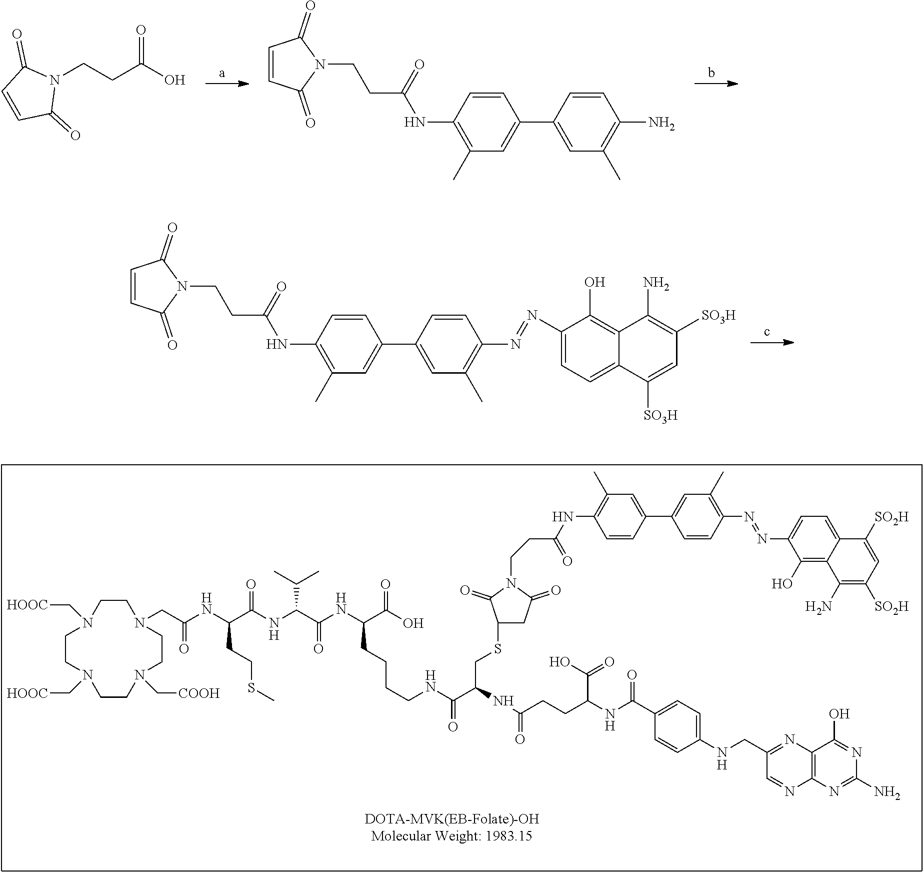

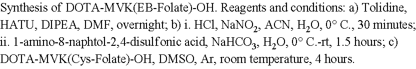

### Example 7

### Synthesis of BBM5

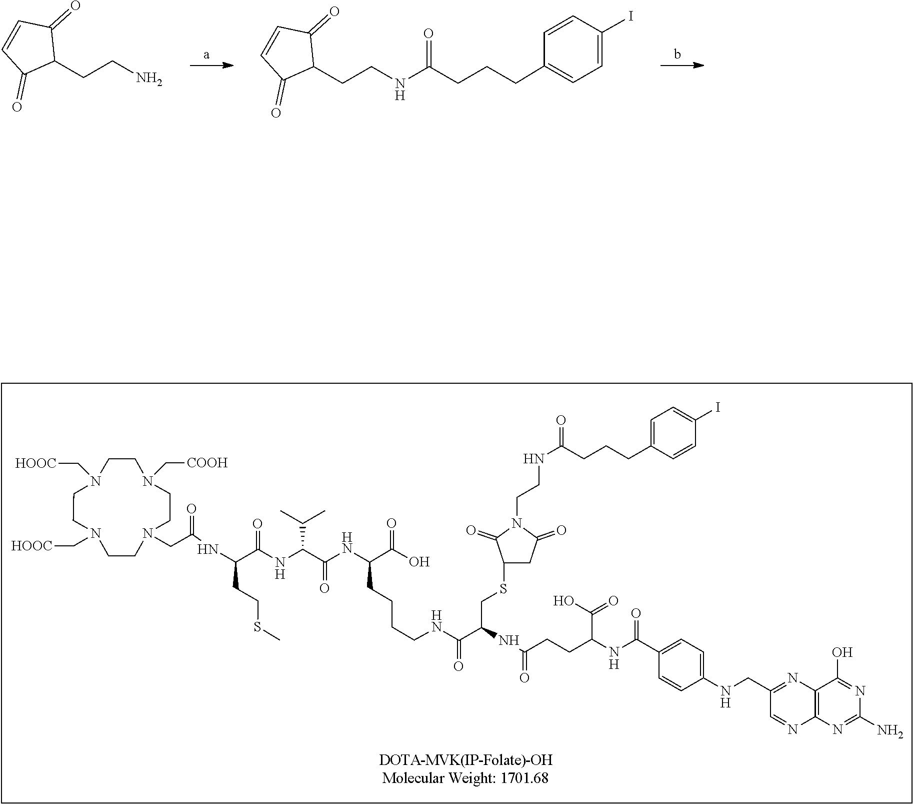 [SMILES: NCCC1C(=O)C=CC1=O ; O=C(CCCc1ccc(I)cc1)NCCC1C(=O)C=CC1=O ; CSCC[C@@H](NC(=O)CN1CCN(CC(=O)O)CCN(CC(=O)O)CCN(CC(=O)O)CC1)C(=O)N[C@@H](C(=O)N[C@H](CCCCNC(=O)[C@@H](CSC1CC(=O)N(CCNC(=O)CCCc2ccc(I)cc2)C1=O)NC(=O)CCC(NC(=O)c1ccc(NCc2cnc3nc(N)nc(O)c3n2)cc1)C(=O)O)C(=O)O)C(C)C]

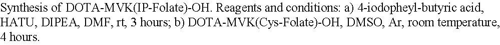

### Example 8

### Validation of Folate-Targeting Via Flow Cytometry

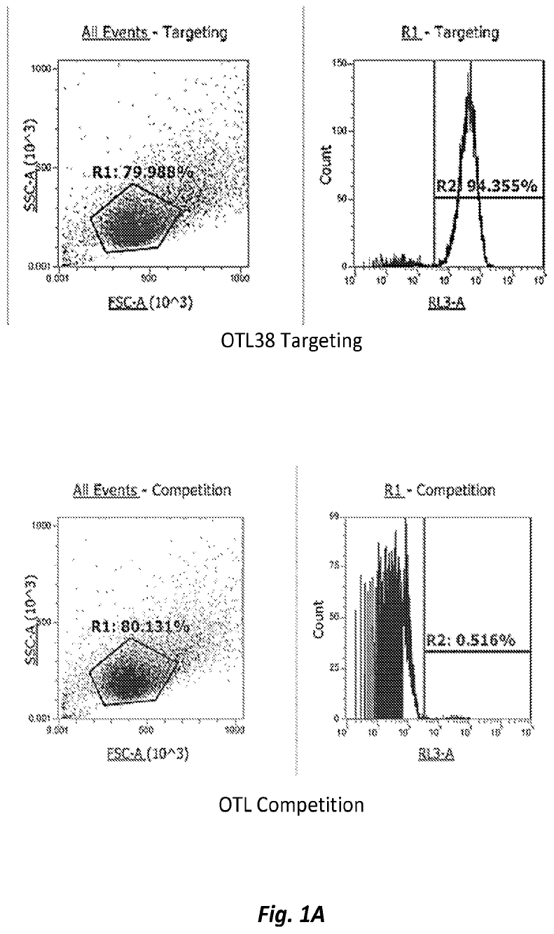

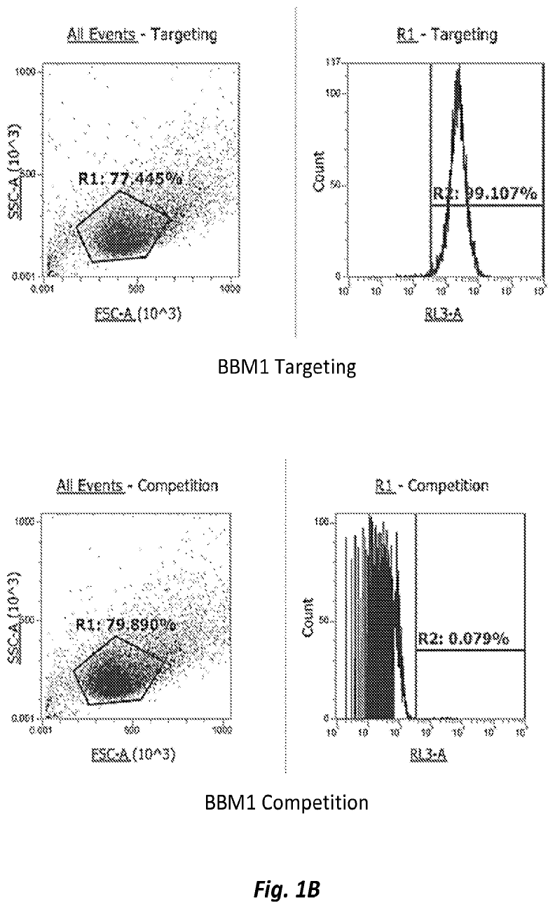

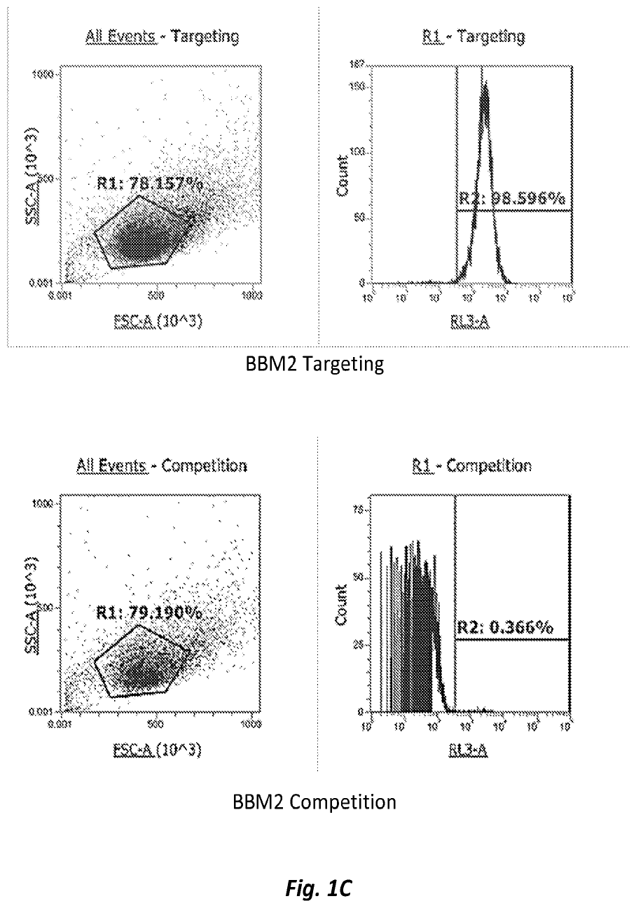

Each of OTL38, BBM1 and BBM2 was incubated with KB cells previously cultured in folate-deficient medium in the absence or presence of 100× excess of folate glucosamine for 1 hour at 4° C. The cells were washed three times and then analyzed via flow cytometry. The results are shown in FIGS. 1A-1C.

### Example 9

### Folate Conjugate Binding Curves

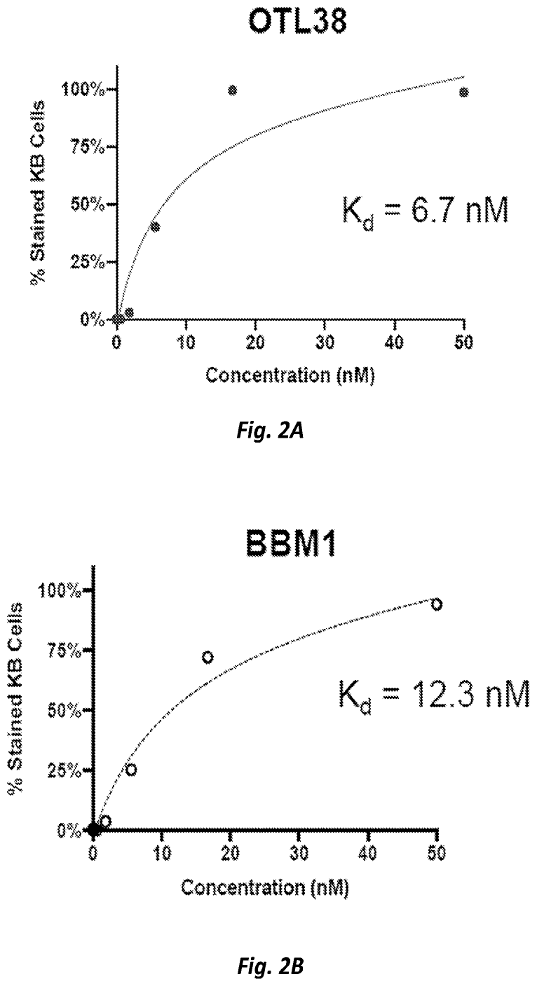

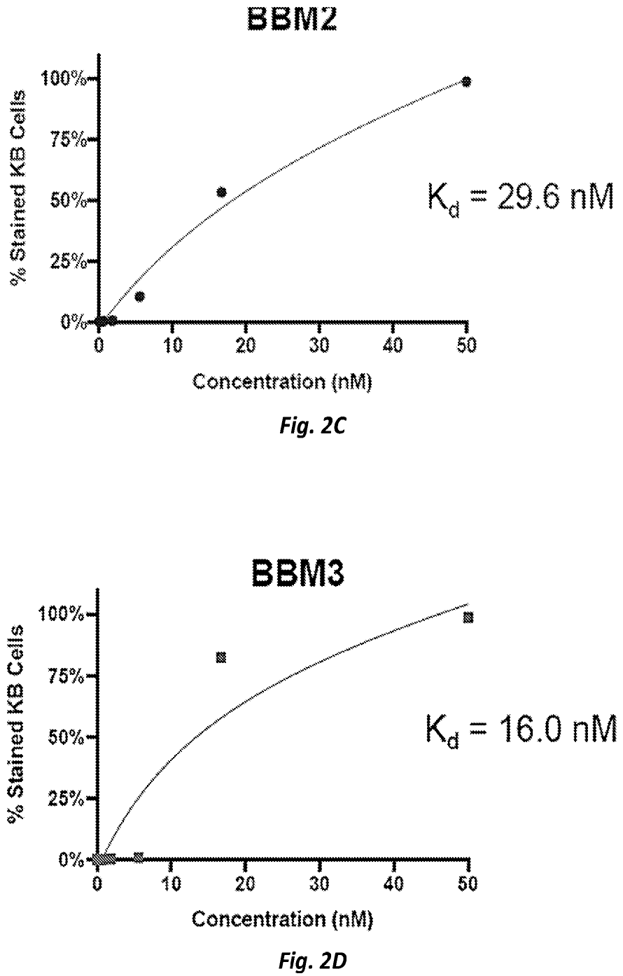

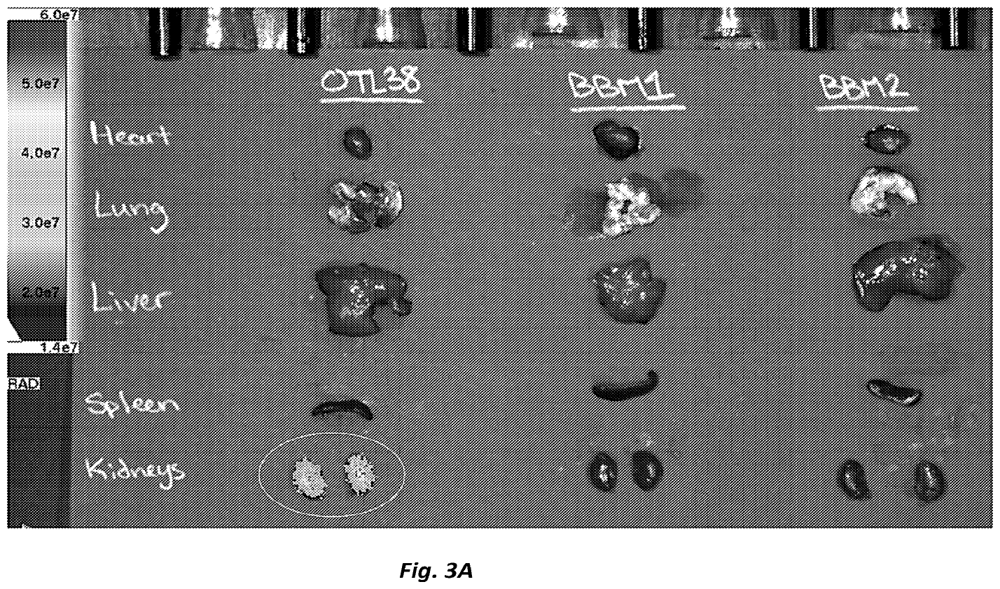

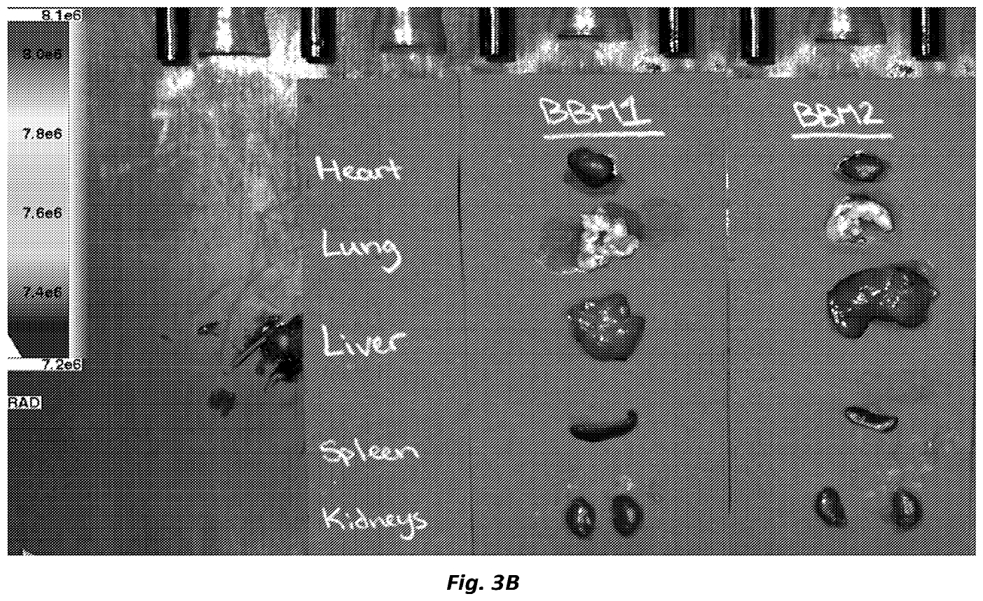

KB cells were incubated for 1 hour at 4° C. with increasing concentrations of OTL38, BBM1, BBM2 or BBM3 in staining buffer, washed three times with staining buffer, suspended in phosphate-buffered saline, and then analyzed via flow cytometry. The results are shown in FIGS. 2A-2D. Concentrations of all conjugates were normalized via fluorescence. All conjugates at 50 nM showed competition with folate-glucosamine.

### Example 10

### In Vivo Studies in Mice

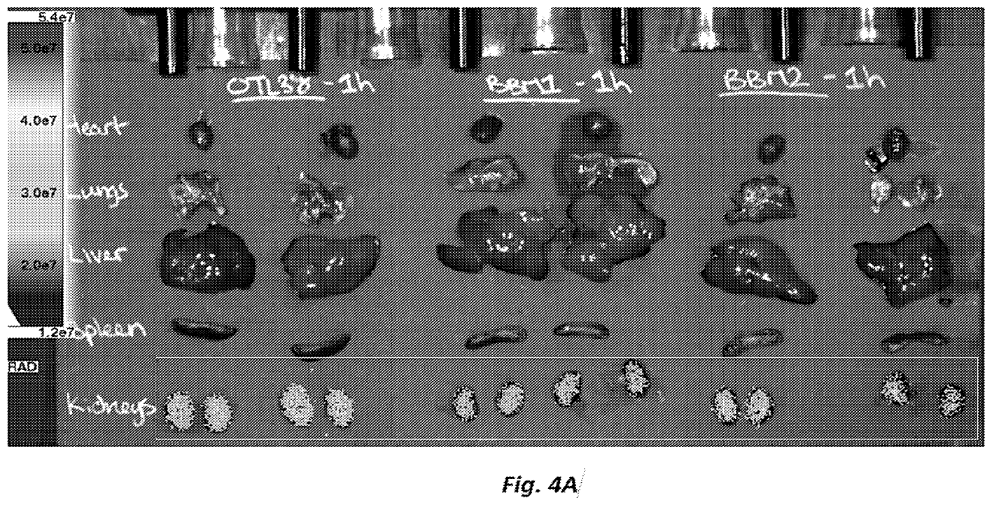

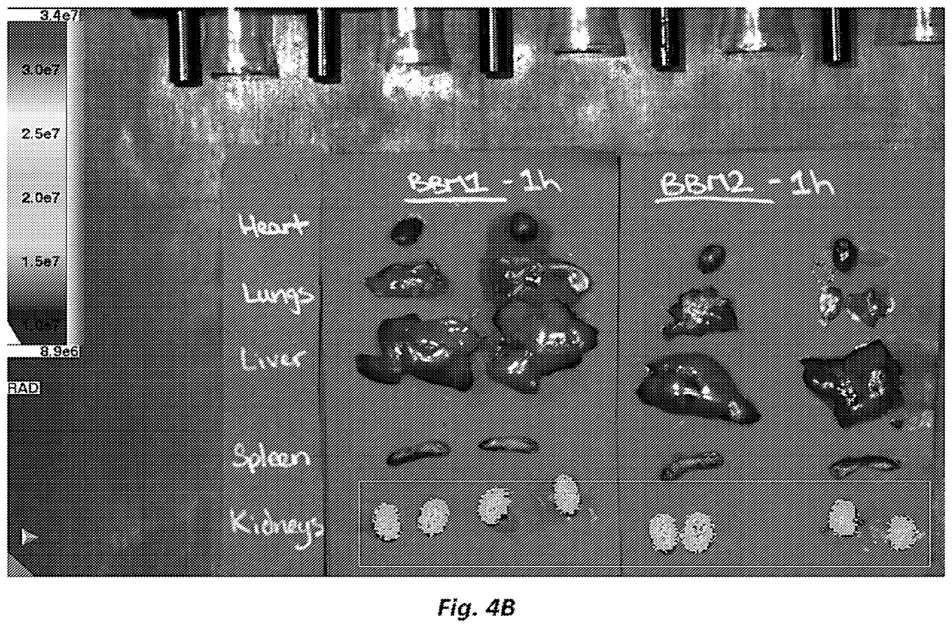

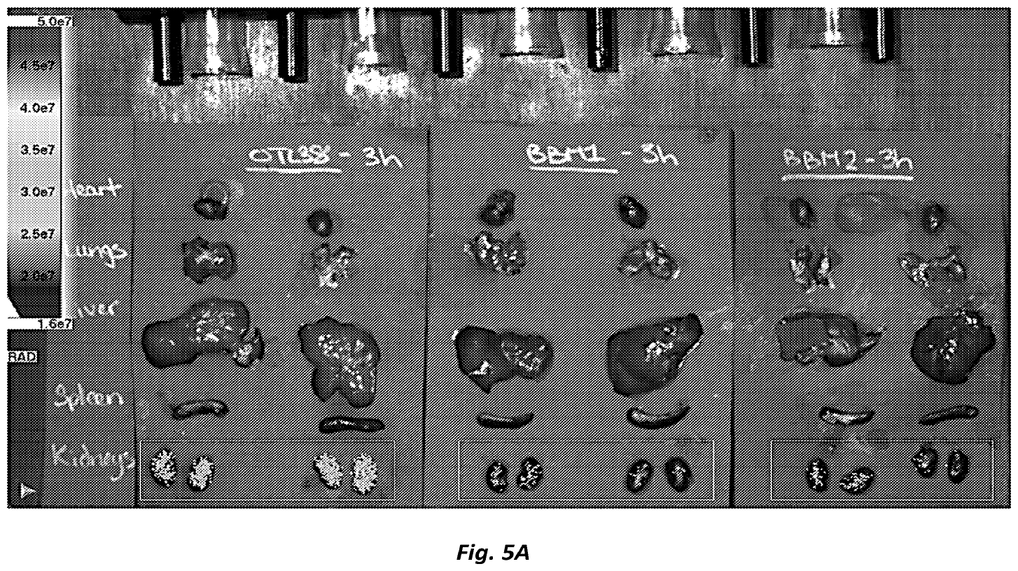

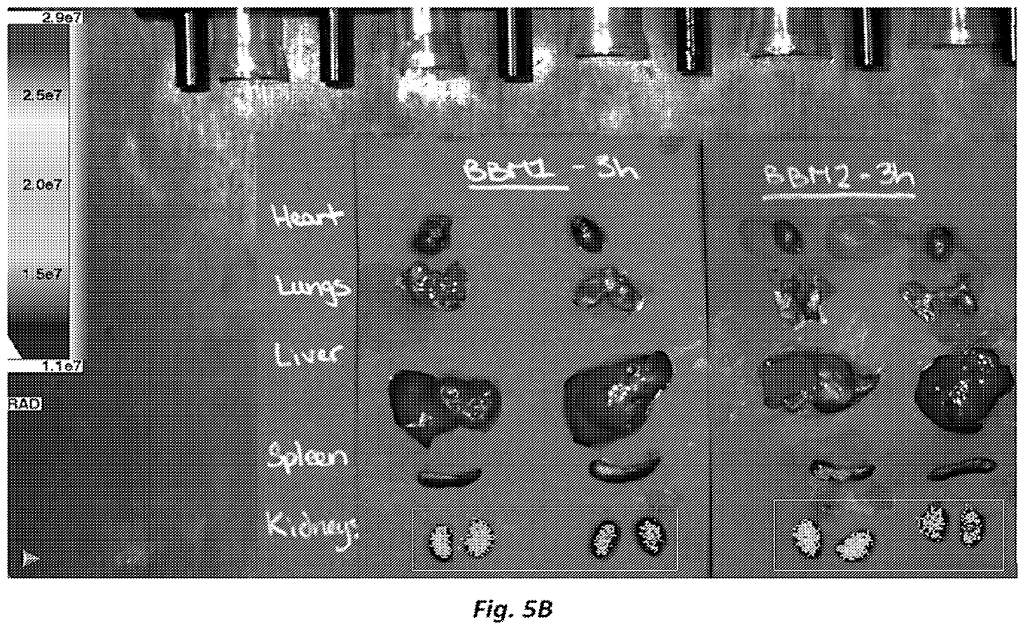

Swiss Webster mice were housed on a folate-deficient diet for 3+ weeks. The mice were then tail vein-injected with 1 nmol of OTL38, BBM1, or BBM2 (1-2 mice/conjugate) and sacrificed at 24 hours, 1 hour, 3 hours, or 6 hours post-injection. The mice were dissected and their hearts, lungs, livers, spleens, and kidneys were scanned for fluorescence. Results are shown in FIGS. 3A and 3B, 4A and 4B, 5A and 5B, and 6A and 6B, respectively.

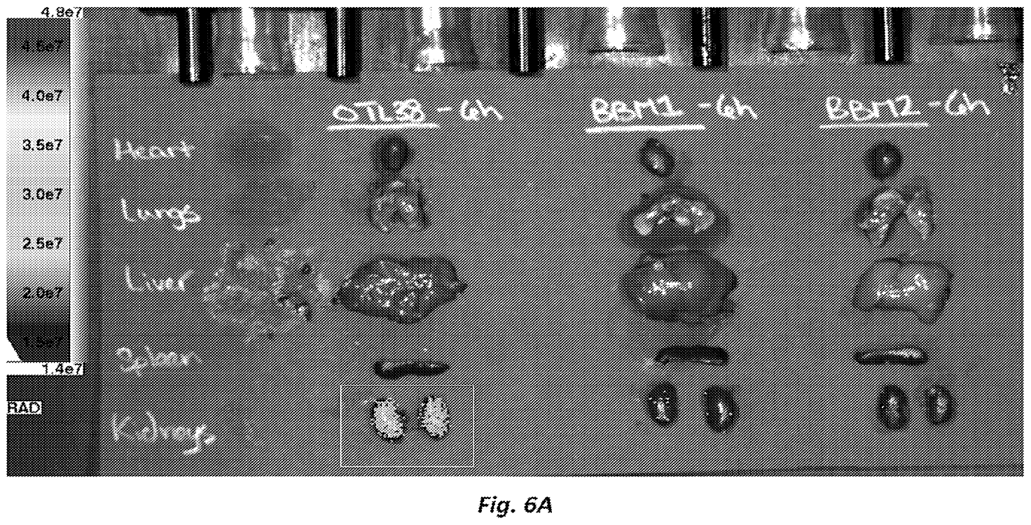

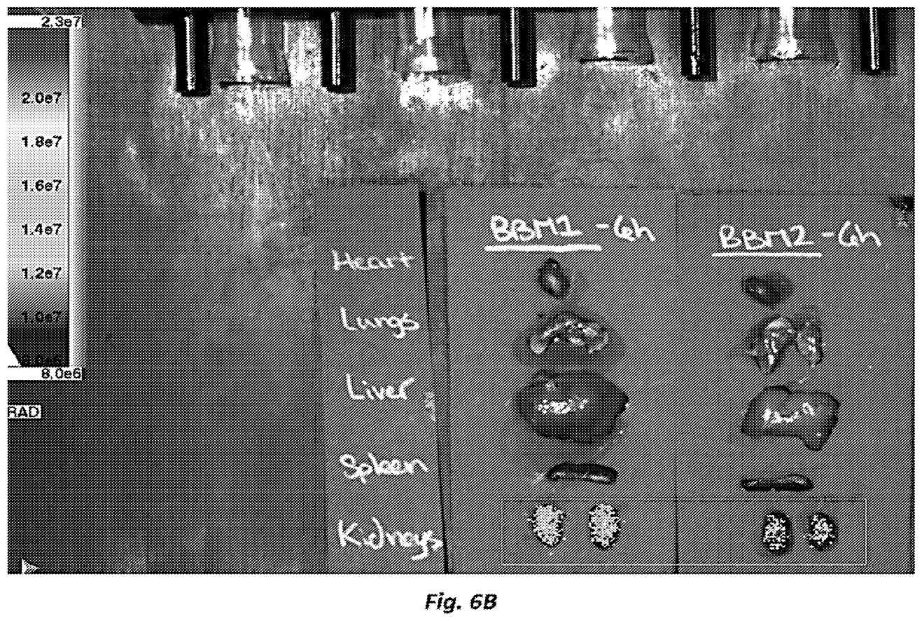

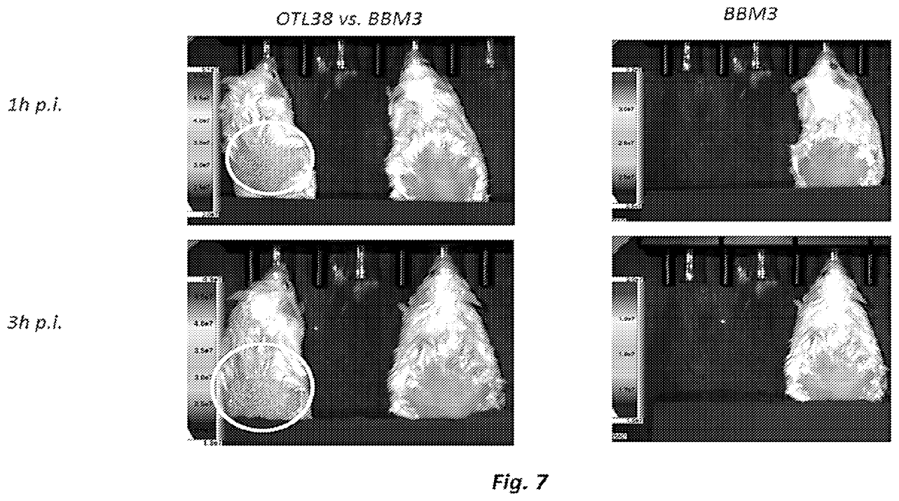

Separately, other Swiss Webster mice were housed on a folate-deficient diet for 3+ weeks and tail vein-injected with 1 nmol of OTL38, BBM1, BBM2 or BBM3 (1 mouse/conjugate). At 1 hour (1 h p.i.) and 3 hours (3 h p.i.) post-injection, the mice were scanned for fluorescence. Results for OTL38 and BBM3 are shown in FIG. 7. The mice were dissected 3 hours post-injection and their hearts, lungs, livers, spleens, and kidneys were scanned for fluorescence. Results for OTL38, BBM1, BBM2 and BBM3 are shown in FIGS. 8A and 8B.

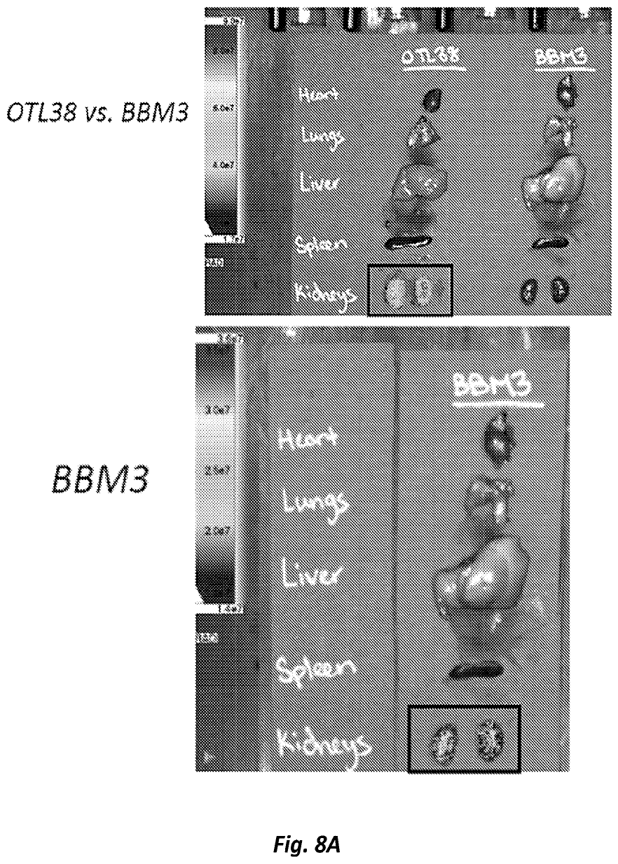

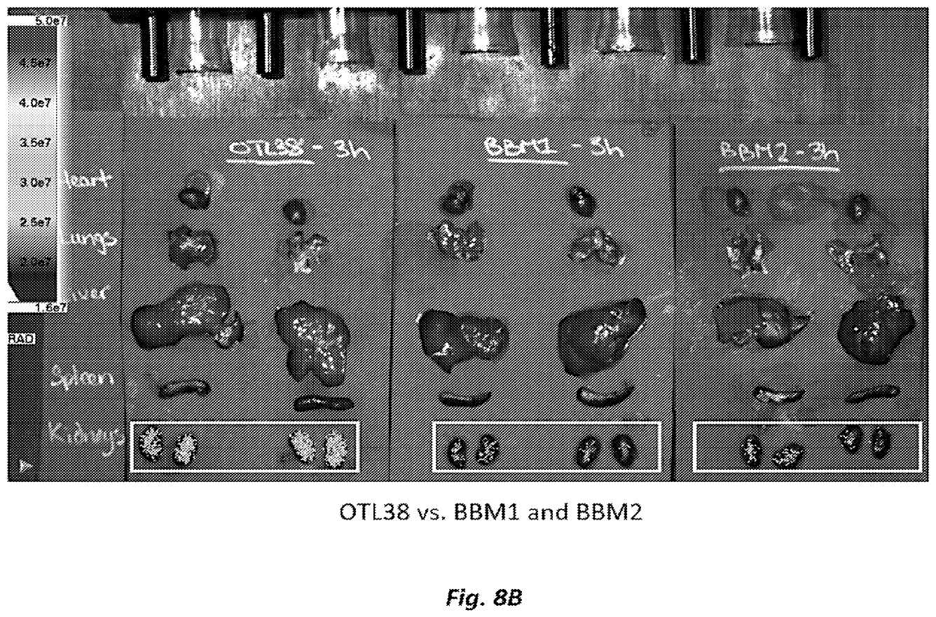

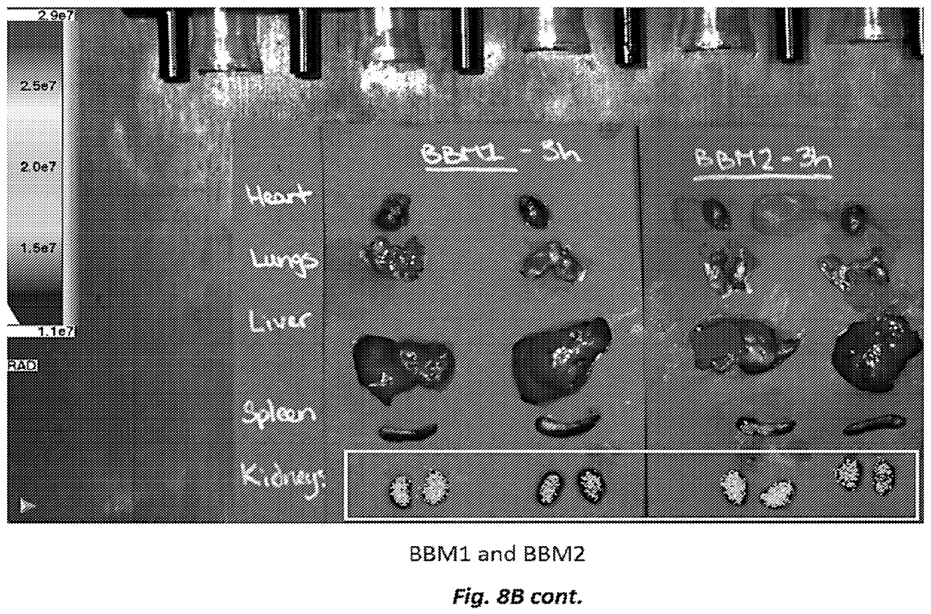

In yet another experiment, Swiss Webster mice were housed on a folate-deficient diet for 3+ weeks and tail vein-injected with 5 nmol of OTL38 or BBM3 (1 mouse/conjugate). At 1 hour (1 h p.i.), 3 hours (h p.i.), 6 hours (6 h p.i.) and 24 hours (24 h p.i.) post-injection, the mice were scanned for fluorescence. The results are shown in FIGS. 9A and 9B. The mice were dissected 24 hours post-injection and their hearts, lungs, livers, spleens and kidneys were scanned for fluorescence. Results are shown in FIG. 10.

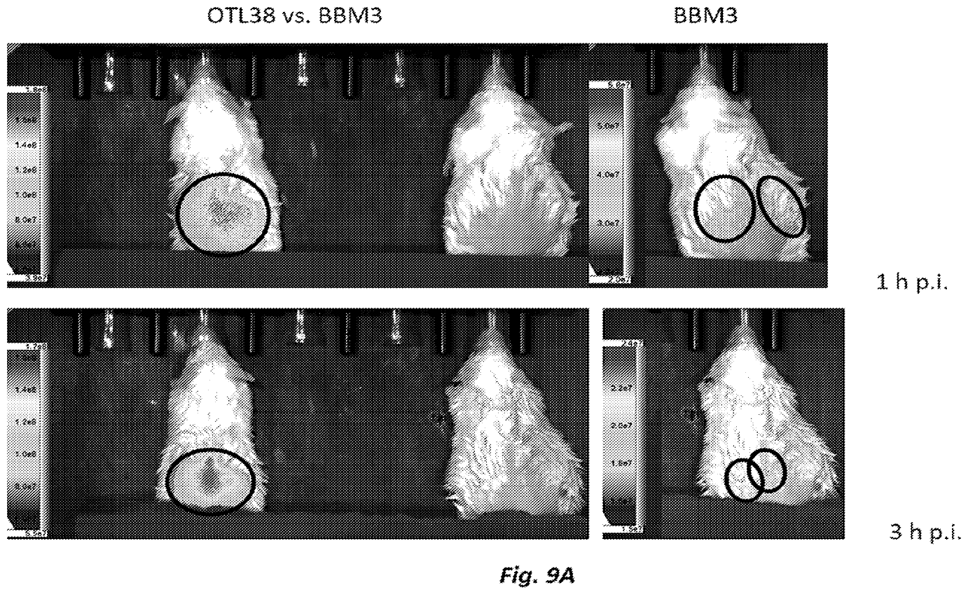

In still yet another experiment, athymic nude mice housed on a folate-deficient diet for 3+ weeks were tail vein-injected with 5 nmol of OTL38, BBM1 or BBM2 (1 mouse/conjugate). At 24 hours post-injection, the mice were dissected and their tumors (KB), hearts, lungs, livers, and kidneys were scanned for fluorescence. Results are shown in FIG. 11.

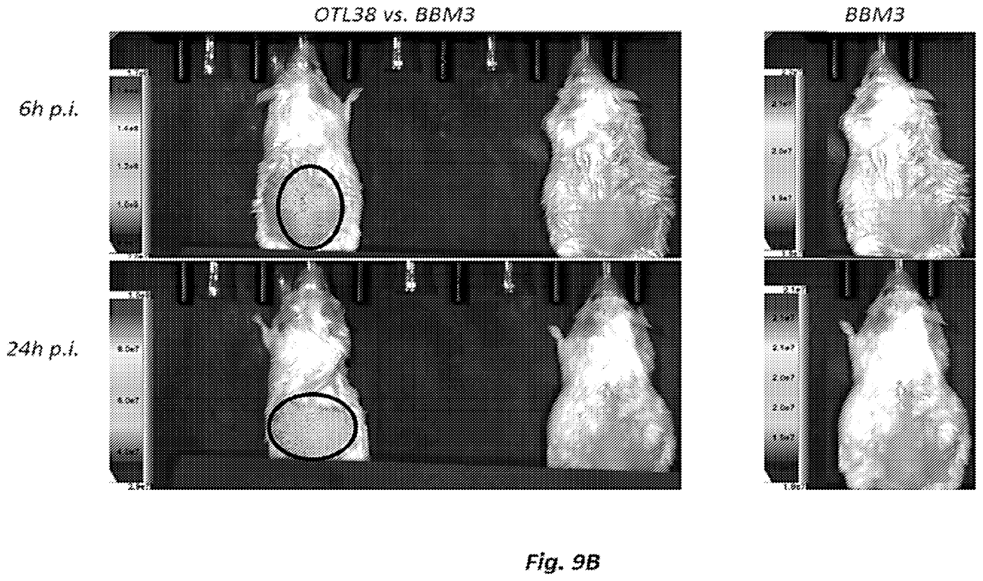

Athymic nude mice housed on a folate-deficient diet for 3+ weeks were tail vein-injected with 5 nmol of OTL38 or BBM3 (1-2 mice/conjugate). At 1 hour (1 hour p.i.), 3 hours (3 hours p.i.), 6 hours (6 hours p.i.), and 24 hours (24 hours p.i.) post-injection, the mice were scanned for fluorescence in the tumor (MDA-MB-231) vs. the kidney. Results are shown in FIG. 12.

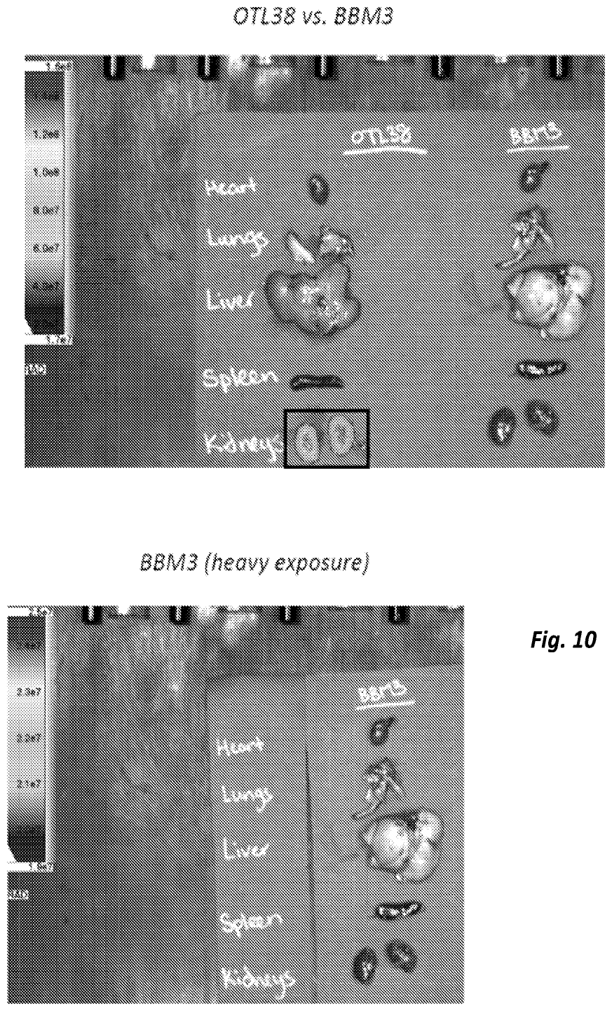

Athymic nude mice were housed on a folate-deficient diet for 3+ weeks and tail vein-injected with 5 nmol of OTL38 or BBM3 (1-2 mice/conjugate). At 1 hour (1 hour p.i.), 3 hours (3 hours p.i.), 6 hours (6 hours p.i.), and 24 hours (24 hours p.i.) post-injection, the mice were scanned for fluorescence in the tumor (MDA-MB-231) vs. the kidney. Results are shown in FIG. 13.

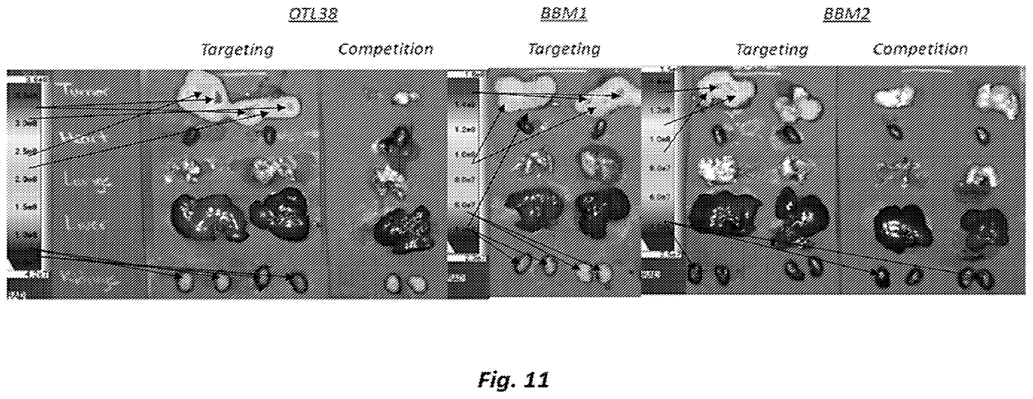

Athymic nude mice were housed on a folate-deficient diet for 3+ weeks and tail vein-injected with 5 nmol of OTL38 or BBM3 (1-2 mice/conjugate). At 24 hours post-injection, the mice were dissected and their tumors (MDA-MB-231), hearts, lungs, livers, spleens, and kidneys were scanned for fluorescence. Results are shown in FIG. 14.

### Example 11

### Radiolabeling of BBM4 and BBM5 with ¹¹¹In

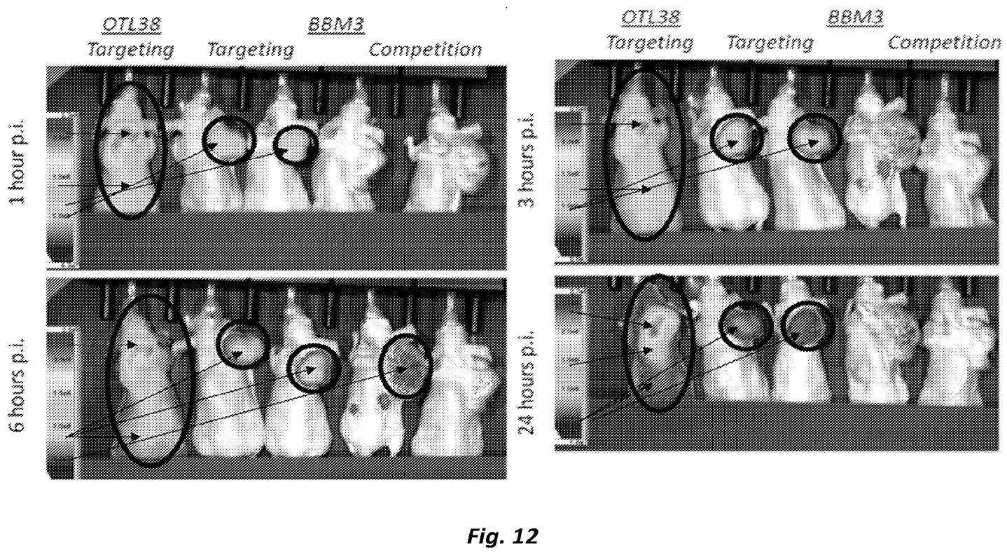

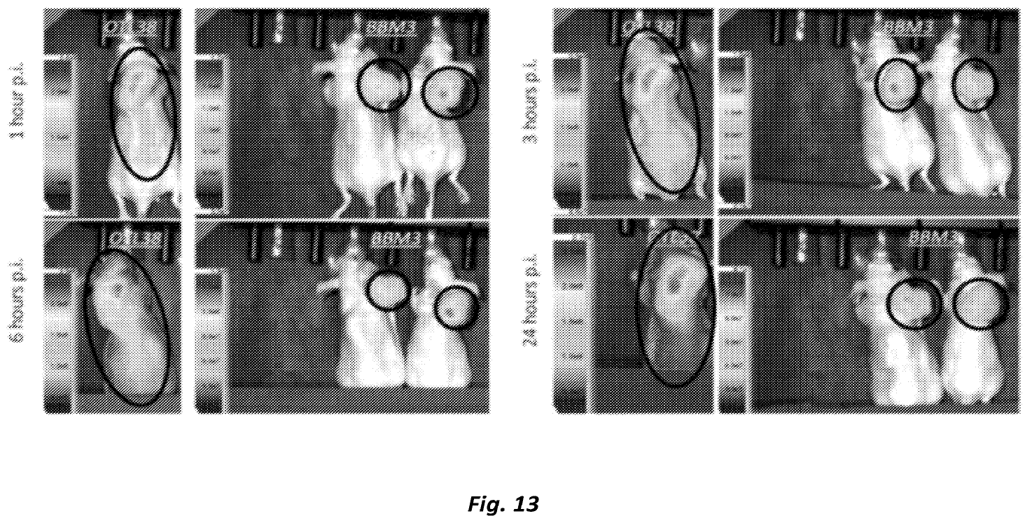

Folate-DOTA conjugates were diluted with ammonium acetate (0.5 M, pH 8.0) to reach a final conjugate concentration of 0.5 mM. Indium-111 (¹¹¹InCl₃) was added to obtain a specific activity of 1 MBq/nmol, then heated to 90° C. for 10 minutes. Sodium-diethylenetriamine pentaacetate solution (5 mM, pH 7.0) was added to complex any unreacted traces of radioactive isotope. Radiochemical purities were analyzed by radio-HPLC with an Agilent 1260 Infinity II with a Flow-RAM detector purchased from LabLogic Systems Ltd. (Brandon, FL) and a reverse-phase XBridge Shield RP18 column (3.0×50 mm, 3.5 μm). The mobile phase consisted of a 20 mM ammonium acetate aqueous buffer (pH 7) (A) and acetonitrile (B) with a linear gradient from 5% B to 95% B over 15 minutes. Radiochemical purities were ≥95% for ¹¹¹In radiolabeled Folate-DOTA conjugates. FIG. 15 is a radio-chromatogram of ¹¹¹In-BBM4. FIG. 16 is a radio-chromatogram of ¹¹¹In-BBM5.

### Example 12

### Binding of ¹¹¹In-BBM4 and ¹¹¹In-BBM5 in KB Cells

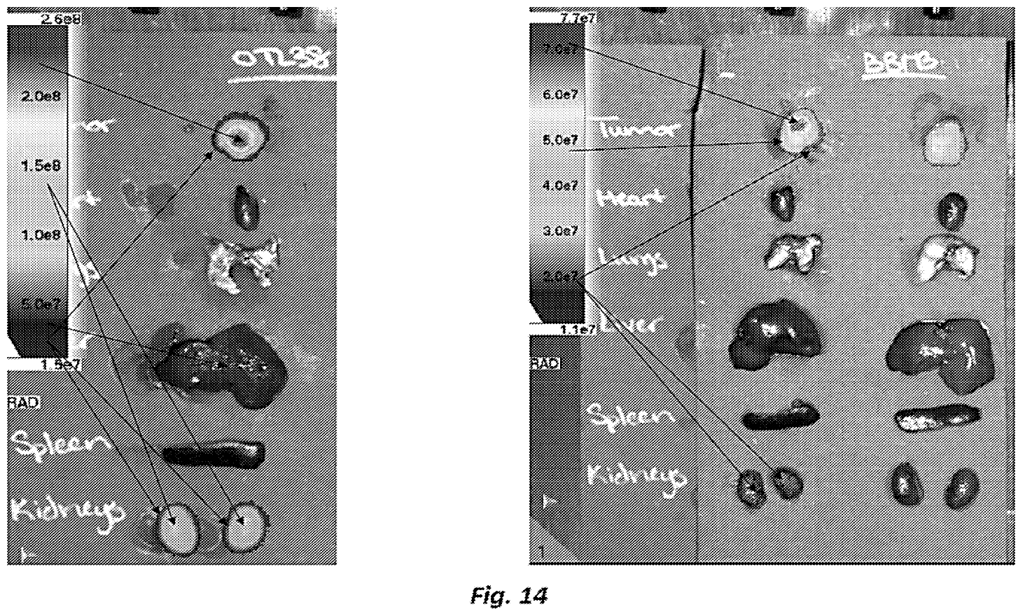

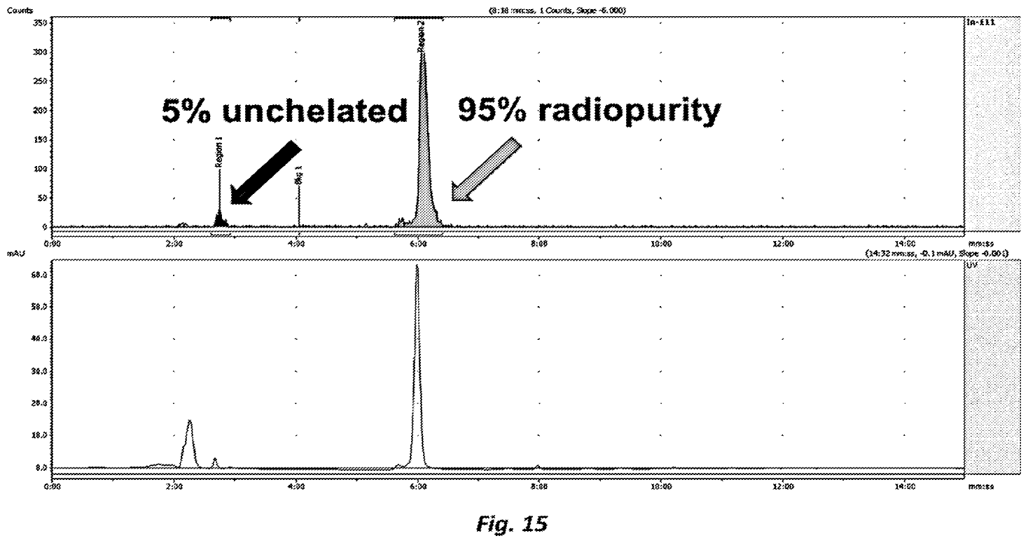

KB cells (200,000) were seeded into 24-well amine coated plates and allowed to adhere overnight. Once the cells reached confluency, they were incubated with increasing concentrations of ¹¹¹In-BBM4 or ¹¹¹In-BBM5 in the absence or presence of 100× excess of folate-glucosamine for one hour at room temperature. After incubation, the cells were washed 3× with PBS to remove unbound radioactivity and dissolved in 1.0 M NaOH. The samples were transferred to tubes and the cell-bound radioactivity was measured using a Packard Cobra Gamma Counter. A specific binding constant was calculated using one-site specific nonlinear fit. FIG. 17A is a graph of concentration (nM) vs. CPM, which shows the binding of Indium-111 radiolabeled BBM4 conjugates in KB cells. FIG. 17B is a graph of concentration (nM) vs. CPM, which shows the binding of Indium-111 radiolabeled BBM5 conjugates in KB cells.

### Example 13

### Binding of ¹¹¹In-BBM4 and ¹¹¹In-BBM5 In Vivo

Radioactive scans were performed with a VECTor/CT system with a clustered multi-pinhole high-energy collimator (MILabs B.V., Utrecht, The Netherlands) of the Bindley Bioscience Center of Purdue University. 12-week-old female ND4 swiss webster mice and 12-week-old female athymic nu/nu mice were purchased from Envigo (Indianapolis, IN). All mice were given access to a folate-deficient diet and water ad libitum. The mice were housed on a standard 12-hour light-dark cycle. MDA-MB-231 tumors were allowed to grow to approximately 1 cm³ by inoculating their shoulder with 5×10⁶ MDA-MB-231 cells before conducting SPECT/CT scans. Each mouse was injected intravenously via tail vein with up to 5 nmol ¹¹¹In-BBM4 or ¹¹¹In-BBM5. Mice (n=1 per group) were anesthetized with 3% isoflurane in oxygen and whole-body scans were performed at multiple time points. The emission scan was conducted for 15-60 minutes. The CT scans were acquired with an X-ray source set at 60 kV and 615 μA. The SPECT images were reconstructed with U-SPECT II software and ¹¹¹In γ-energy windows of 171 and 241 keV. A POS-EM algorithm was used with 16 subsets and 4 iterations on a 0.8 mm voxel grid. The CT images were reconstructed using NRecon software. The datasets were fused and filtered using PMOD software (version 3.2).

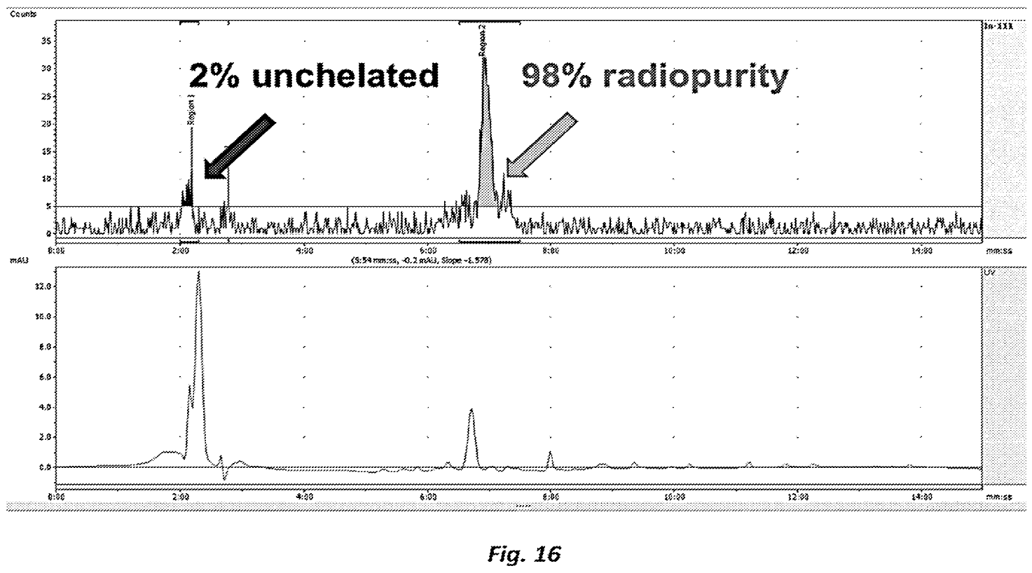

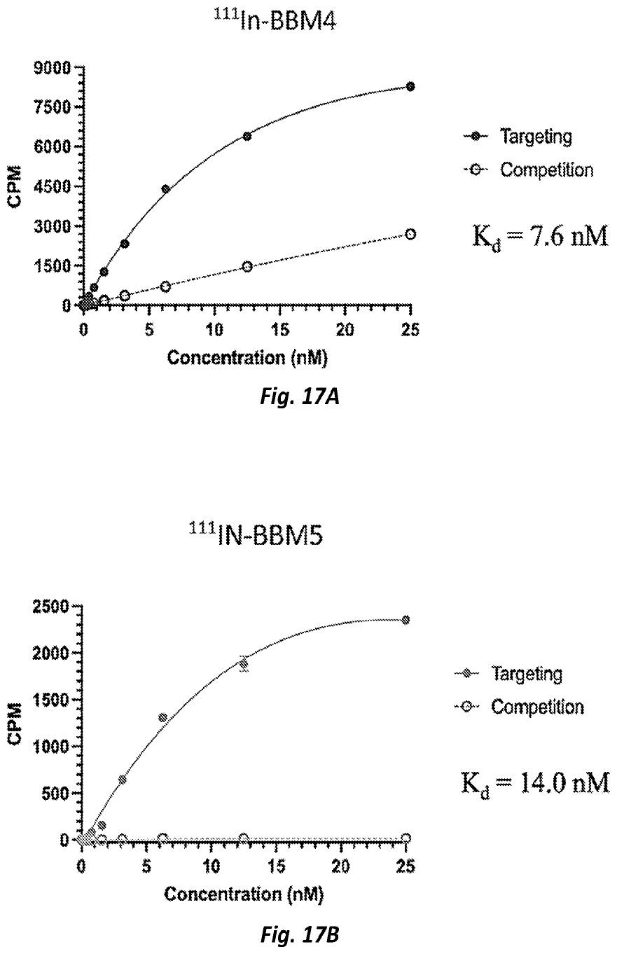

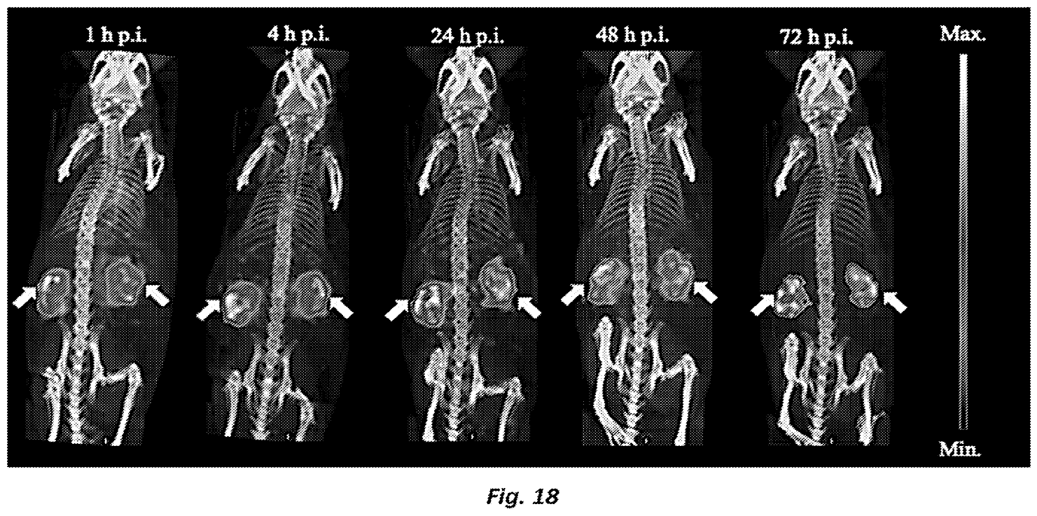

FIG. 18 shows the SPECT/CT images of Indium-111 radiolabeled BBM4 conjugates (¹¹¹In-BBM4) in a healthy mouse. 1 nmol injected radiolabeled with ˜250 uCi of In-111. Yellow arrows indicate kidneys. FIG. 19 shows the SPECT/CT images of Indium-111 radiolabeled BBM5 conjugates (¹¹¹In-BBM5) in a healthy mouse. 1 nmol injected radiolabeled with ˜250 uCi of In-111. Yellow arrows indicate kidneys. FIG. 20 shows the SPECT/CT images of Indium-111 radiolabeled BBM4 conjugates (¹¹¹In-BBM4) in a healthy mouse. 5 nmol injected radiolabeled with ˜250 uCi of In-111. Yellow arrows indicate kidneys.

### Example 14

### Radiolabeling of BBM4 and BBM5 with ¹⁷⁷Lu

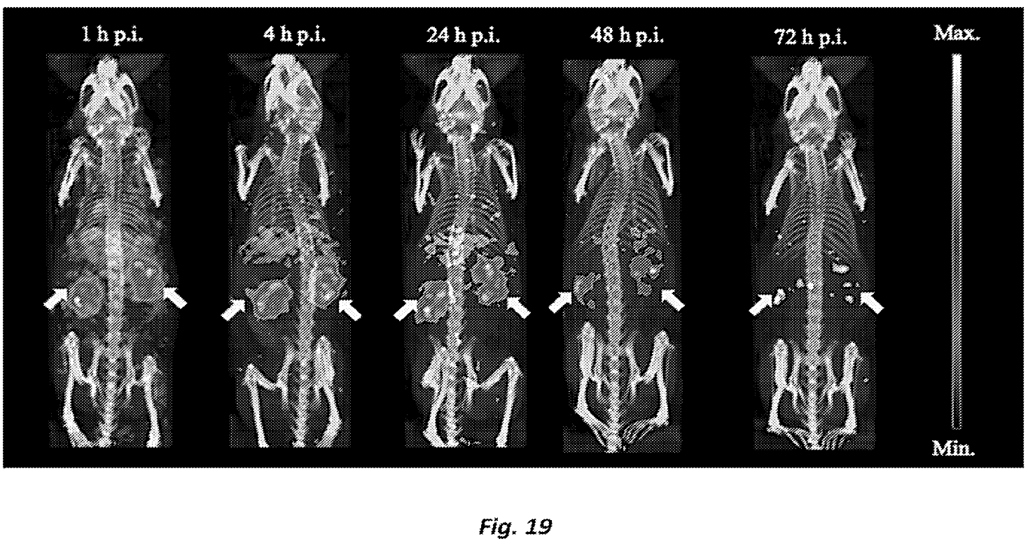

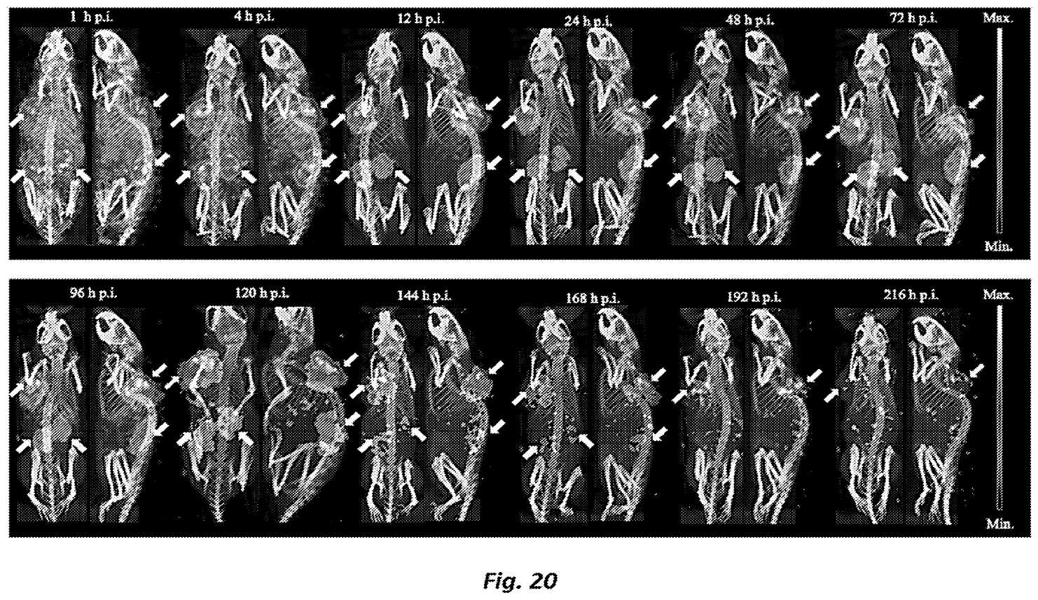

Folate-DOTA conjugates were diluted with ammonium acetate (0.5 M, pH 8.0) to reach a final conjugate concentration of 0.5 mM. Lutetium-177 (¹⁷⁷LuCl₃) was added to obtain a specific activity of 4 MBq/nmol, then heated to 90° C. for 10 minutes. Sodium-diethylenetriamine pentaacetate solution (5 mM, pH 7.0) was added to complex any unreacted traces of radioactive isotope. Radiochemical purities were analyzed by radio-HPLC with an Agilent 1260 Infinity II with a Flow-RAM detector purchased from LabLogic (Brandon, FL) and a reverse-phase XBridge Shield RP18 column (3.0×50 mm, 3.5 μm). The mobile phase consisted of a 20 mM ammonium acetate aqueous buffer (pH 7) (A) and acetonitrile (B) with a linear gradient from 5% B to 95% B over 15 minutes. Radiochemical purities were ≥95% for ¹⁷⁷Lu radiolabeled Folate-DOTA conjugates. FIG. 21 is a radio-chromatogram of ¹⁷⁷Lu-BBM4. FIG. 22 is a radio-chromatogram of ¹⁷⁷Lu-BBM5.

### Example 15

### Binding of ¹⁷⁷Lu-BBM4 and ¹⁷⁷Lu-BBM5 In Vivo

12-week-old female athymic nu/nu mice were purchased from Envigo and given access to a folate-deficient diet and water ad libitum. The mice were housed on a standard 12-hour light-dark cycle. KB tumors were allowed to grow to approximately 300 mm³ by inoculating their shoulder with 5×10⁶ KB cells. Mice were randomly sorted into control or treatment groups prior to the initiation of the therapy studies. Mice received a single intravenous dose of sterile saline or 5 nmol of BBM4 or BBM5 radiolabeled with 18 MBq of lutetium-177 via the tail vein on day 0. Tumors were measured in two perpendicular directions every other day during therapy, and their volumes were calculated as 0.5×L×W², where L is the longest axis (in millimeters), and W is the axis perpendicular to L (in millimeters). Humane endpoint criteria were defined as weight loss of more than 20% of the initial body weight, a tumor volume of more than 1,800 mm³, or open ulceration. Mice were euthanized on reaching one of the predefined endpoint criteria.

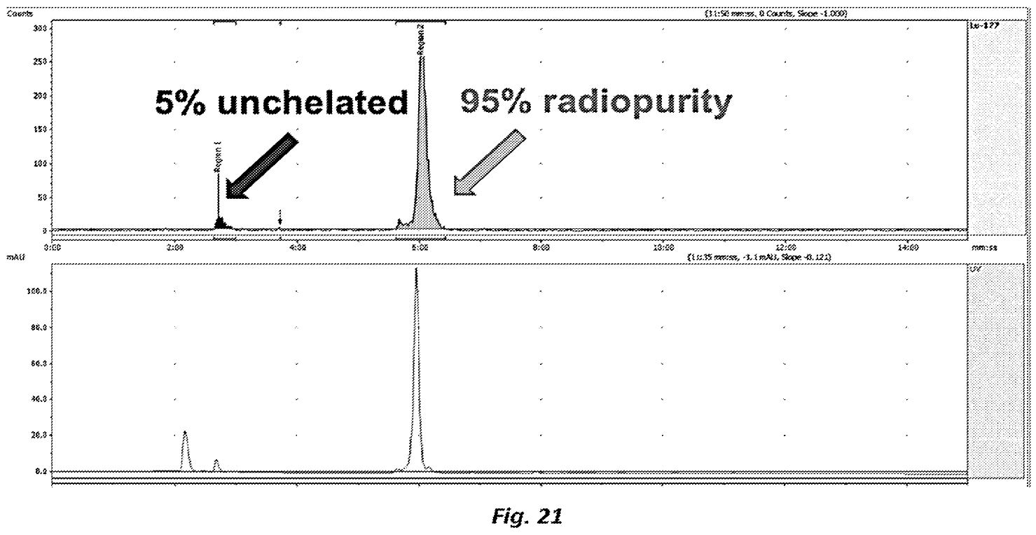

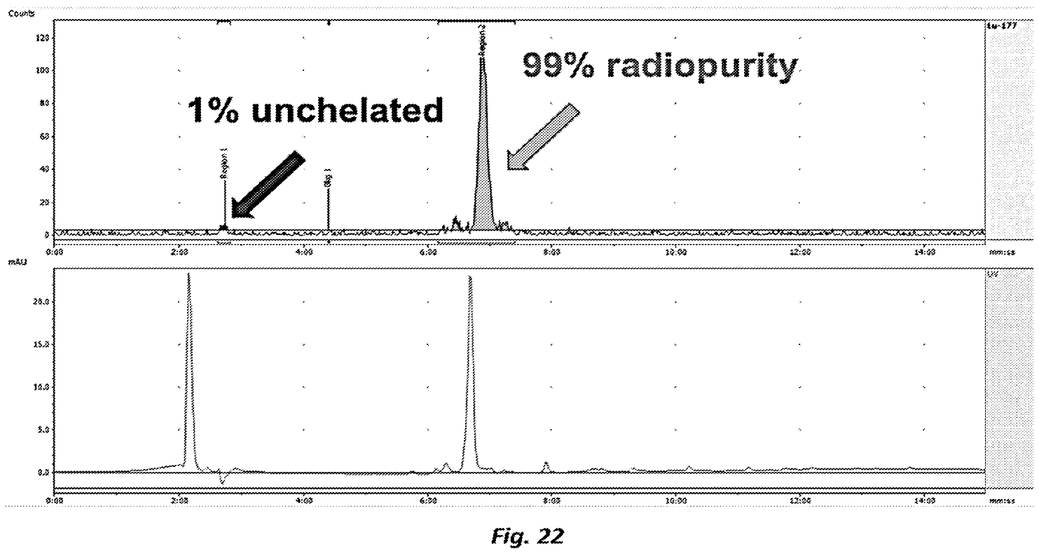

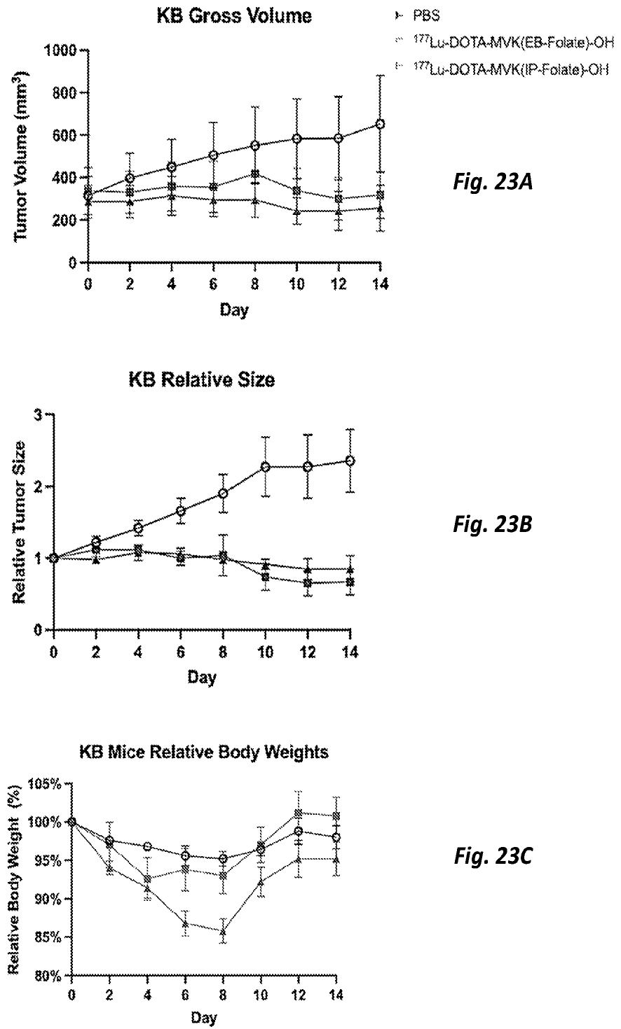

FIG. 23A is a graph of day vs. tumor volume (mm³), which shows the gross volume of KB tumors in mice treated with ¹⁷⁷Lu radiolabeled BBM conjugates. FIG. 23B is a graph of day vs. relative tumor size, which shows the relative size of KB tumors in mice treated with ¹⁷⁷Lu radiolabeled BBM conjugates. FIG. 23C is a graph of day vs. relative body weight (%), which shows the relative body weights of mice with KB tumors and treated with ¹⁷⁷Lu radiolabeled BBM conjugates.

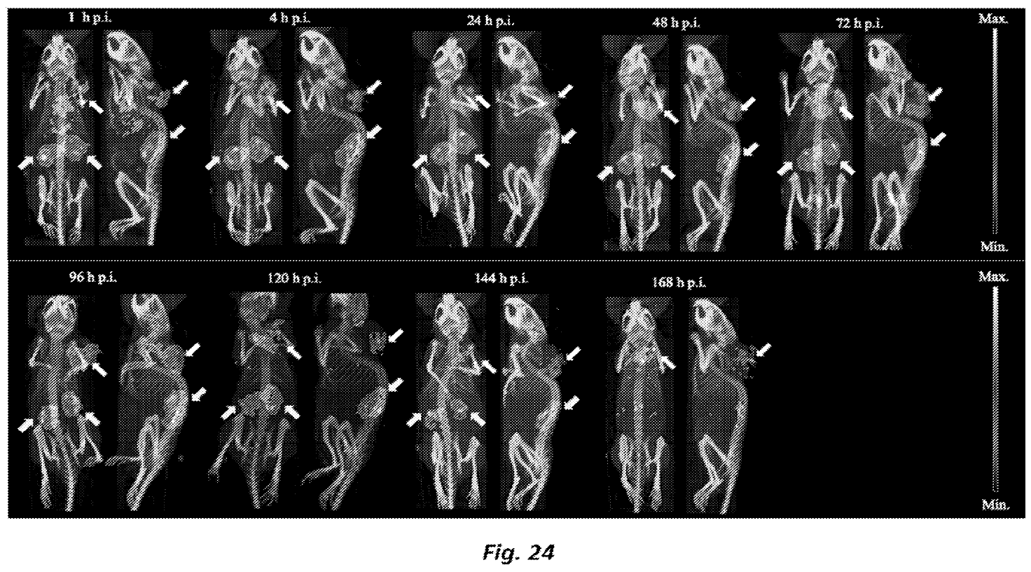

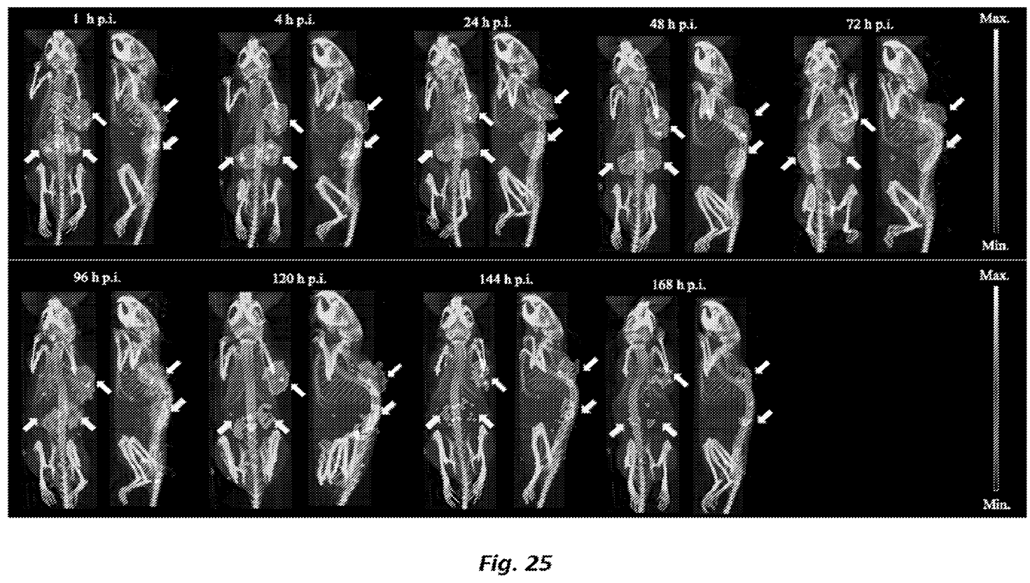

Radioactive scans were performed with a VECTor/CT system with a clustered multi-pinhole high-energy collimator (MILabs B.V., Utrecht, The Netherlands) of the Bindley Bioscience Center of Purdue University. The mice scanned were selected from the radiotherapy-treated groups in FIGS. 23A-23C. Mice (n=1 per group) were anesthetized with 3% isoflurane in oxygen and whole-body scans were performed at multiple time points. The emission scan was conducted for 15-60 minutes. The CT scans were acquired with an X-ray source set at 60 kV and 615 μA. The SPECT images were reconstructed with U-SPECT II software and ¹⁷⁷Lu γ-energy window of 208 keV. A POS-EM algorithm was used with 16 subsets and 4 iterations on a 0.8 mm voxel grid. The CT images were reconstructed using NRecon software. The datasets were fused and filtered using PMOD software (version 3.2). FIG. 24 shows the SPECT/CT images of a mouse treated with ¹⁷⁷Lu radiolabeled BBM4 conjugate. 5 nmol injected radiolabeled with ˜500 uCi of Lu-177. Superior arrow indicates tumor, inferior arrow indicates kidney. FIG. 25 shows the SPECT/CT images of a mouse treated with ¹⁷⁷Lu radiolabeled BBM5 conjugate. 5 nmol injected radiolabeled with ˜500 uCi of Lu-177. Superior arrow indicates tumor, inferior arrow indicates kidney.

### Example 16

### Folate Dye Conjugate Structures with BBM Enzyme-Labile Linkers

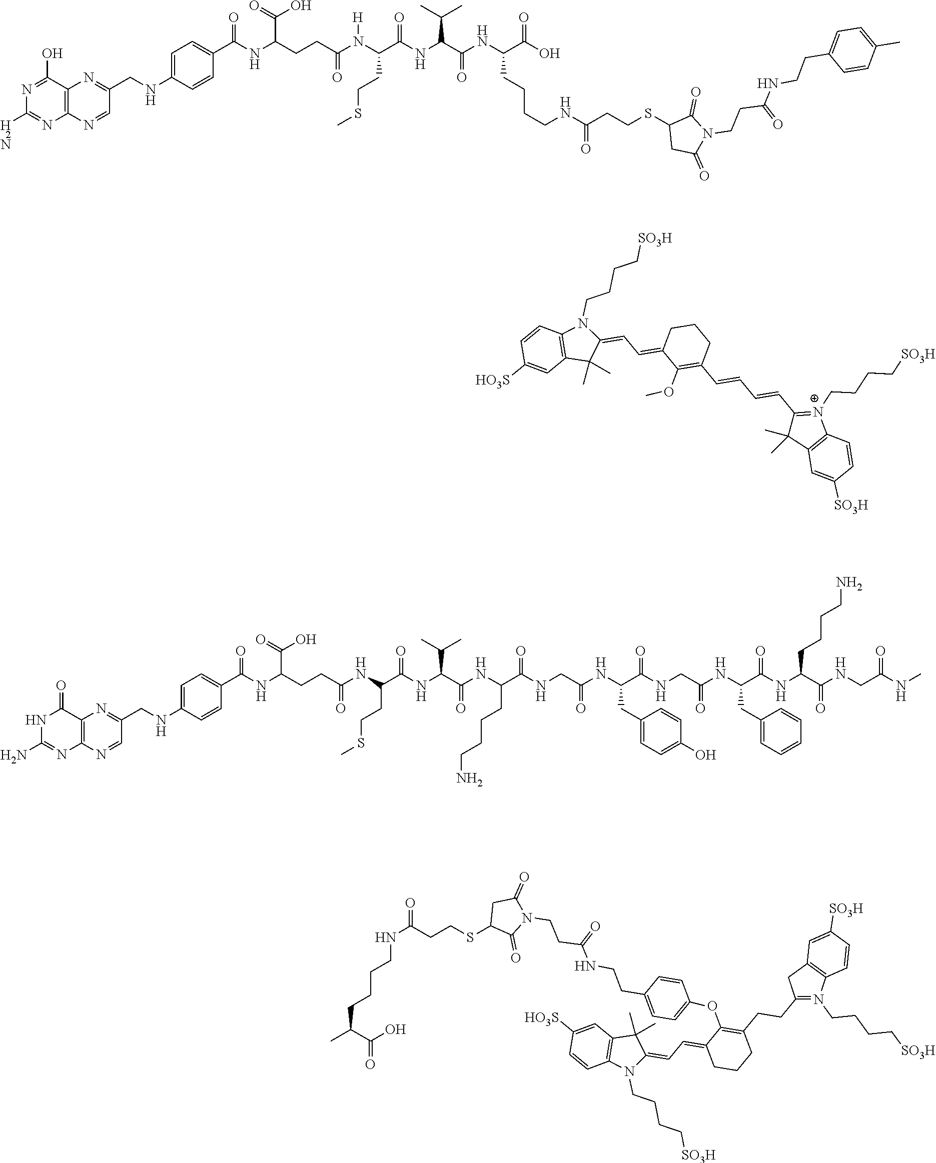

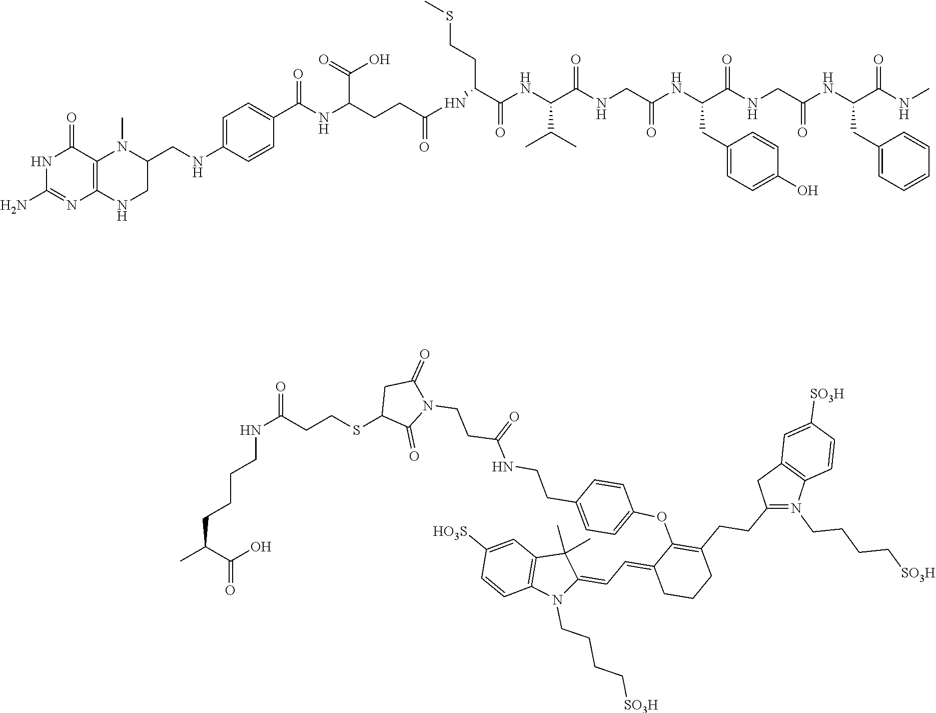

### Example 17

### Folate Radio-Conjugate Structures with BBM Enzyme-Labile Linkers

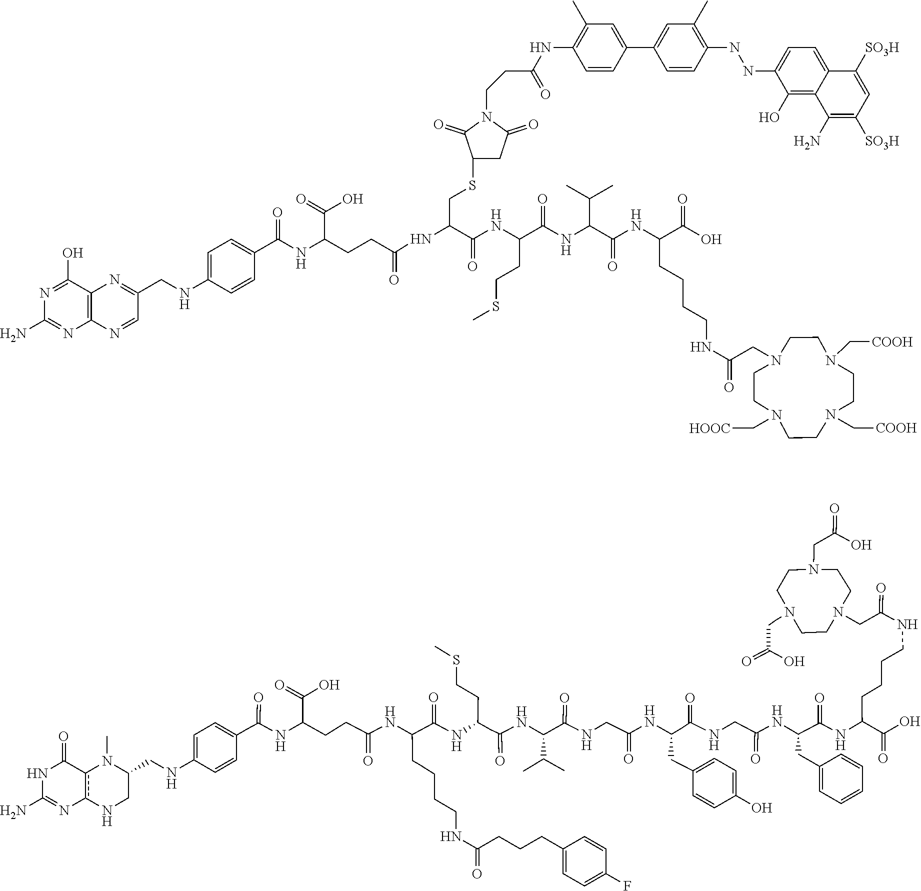

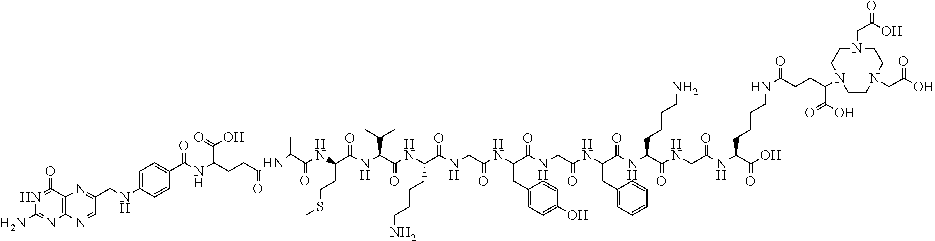

### Other References

1. Weitman, S. D.; Lark, R. H.; Coney, L. R.; Fort, D. W.; Frasca, V.; Zurawski, V. R.; Kamen, B. A., Distribution of the Folate Receptor GP38 in Normal and Malignant Cell Lines and Tissues. Cancer Research 1992, 52 (12), 3396.
2. Coney, L. R.; Tomassetti, A.; Carayannopoulos, L.; Frasca, V.; Kamen, B. A.; Colnaghi, M. I.; Zurawski, V. R., Cloning of a Tumor-associated Antigen: MOv18 and MOv19 Antibodies Recognize a Folate-binding Protein. Cancer Research 1991, 51 (22), 6125.
3. Garin-Chesa, P.; Campbell, I.; Saigo, P. E.; Lewis, J. L., Jr.; Old, L. J.; Rettig, W. J., Trophoblast and ovarian cancer antigen LK26. Sensitivity and specificity in immunopathology and molecular identification as a folate-binding protein. The American journal of patholog 1993, 142 (2), 557-567.
4. Parker, N.; Turk, M. J.; Westrick, E.; Lewis, J. D.; Low, P. S.; Leamon, C. P., Folate receptor expression in carcinomas and normal tissues determined by a quantitative radioligand binding assay. Analytical Biochemistry 2005, 338 (2), 284-293.
5. Low, P. S.; Henne, W. A.; Doorneweerd, D. D., Discovery and Development of Folic-Acid-Based Receptor Targeting for Imaging and Therapy of Cancer and Inflammatory Diseases. Accounts of Chemical Research 2008, 41 (1), 120-129.
6. Low, P. S.; Kularatne, S. A., Folate-targeted therapeutic and imaging agents for cancer. Current Opinion in Chemical Biology 2009, 13 (3), 256-262.
7. Holm, J.; Hansen, S. I.; Hoier-Madsen, M.; Bostad, L., A high-affinity folate binding protein in proximal tubule cells of human kidney. Kidney International 1992, 41 (1), 50-55.
8. Birn, H.; Spiegelstein, O.; Christensen, E. I.; Finnell, R. H., Renal Tubular Reabsorption of Folate Mediated by Folate Binding Protein 1. Journal of the American Society of Nephrology 2005, 16 (3), 608.
9. Sandoval, R. M.; Kennedy, M. D.; Low, P. S.; Molitoris, B. A., Uptake and trafficking of fluorescent conjugates of folic acid in intact kidney determined using intravital two-photon microscopy. American Journal of Physiology—Cell Physiology 2004, 287 (2), C517-C526.
10. Zhou, P.; Sun, X.; Zhang, Z., Kidney-targeted drug delivery systems. Acta Pharmaceutica Sinica B 2014, 4 (1), 37-42.
11. Müller, C.; Schibli, R., Prospects in Folate Receptor-Targeted Radionuclide Therapy. Frontiers in Oncology 2013, 3 (249).
12. Müller, C.; Struthers, H.; Winiger, C.; Zhernosekov, K.; Schibli, R., DOTA Conjugate with an Albumin-Binding Entity Enables the First Folic Acid-Targeted 177Lu-Radionuclide Tumor Therapy in Mice. Journal of Nuclear Medicine 2013, 54 (1), 124-131.
13. Farkas, R.; Siwowska, K.; Ametamey, S. M.; Schibli, R.; van der Meulen, N. P.; Müller, C., 64Cu- and 68Ga-Based PET Imaging of Folate Receptor-Positive Tumors: Development and Evaluation of an Albumin-Binding NODAGA-Folate. Molecular Pharmaceutics 2016, 13 (6), 1979-1987.
14. Radford, L. L.; Fernandez, S.; Beacham, R.; El Sayed, R.; Farkas, R.; Benesovi, M.; Müller, C.; Lapi, S. E., New 55Co-labeled Albumin-Binding Folate Derivatives as Potential PET Agents for Folate Receptor Imaging. Pharmaceuticals 2019, 12 (4).
15. Arano, Y.; Fujioka, Y.; Akizawa, H.; Ono, M.; Uehara, T.; Wakisaka, K.; Nakayama, M.; Sakahara, H.; Konishi, J.; Saji, H., Chemical Design of Radiolabeled Antibody Fragments for Low Renal Radioactivity Levels. Cancer Research 1999, 59 (1), 128-134.
16. Uehara, T.; Koike, M.; Nakata, H.; Hanaoka, H.; Iida, Y.; Hashimoto, K.; Akizawa, H.; Endo, K.; Arano, Y., Design, Synthesis, and Evaluation of [188Re]Organorhenium-Labeled Antibody Fragments with Renal Enzyme-Cleavable Linkage for Low Renal Radioactivity Levels. Bioconjugate Chemistry 2007, 18 (1), 190-198.
17. Akizawa, H.; Uehara, T.; Arano, Y., Renal uptake and metabolism of radiopharmaceuticals derived from peptides and proteins. Advanced Drug Delivery Reviews 2008, 60 (12), 1319-1328.
18. Akizawa, H.; Imajima, M.; Hanaoka, H.; Uehara, T.; Satake, S.; Arano, Y., Renal Brush Border Enzyme-Cleavable Linkages for Low Renal Radioactivity Levels of Radiolabeled Antibody Fragments. Bioconjugate Chemistry 2013, 24 (2), 291-299.
19. Suzuki, C.; Uehara, T.; Kanazawa, N.; Wada, S.; Suzuki, H.; Arano, Y., Preferential Cleavage of a Tripeptide Linkage by Enzymes on Renal Brush Border Membrane To Reduce Renal Radioactivity Levels of Radiolabeled Antibody Fragments. Journal of Medicinal Chemistry 2018, 61 (12), 5257-5268.
20. Uehara, T.; Yokoyama, M.; Suzuki, H.; Hanaoka, H.; Arano, Y., A Gallium-67/68-Labeled Antibody Fragment for Immuno-SPECT/PET Shows Low Renal Radioactivity Without Loss of Tumor Uptake. Clinical Cancer Research 2018, 24 (14), 3309-3316.
21. Uehara, T.; Kanazawa, N.; Suzuki, C.; Mizuno, Y.; Suzuki, H.; Hanaoka, H.; Arano, Y., Renal Handling of 99mTc-Labeled Antibody Fab Fragments with a Linkage Cleavable by Enzymes on Brush Border Membrane. Bioconjugate Chemistry 2020, 31 (11), 2618-2627.
22. Bendre, S.; Zhang, Z.; Kuo, H.-T.; Rousseau, J.; Zhang, C.; Merkens, H.; Roxin, Å.; Bénard, F.; Lin, K.-S., Evaluation of Met-Val-Lys as a Renal Brush Border Enzyme-Cleavable Linker to Reduce Kidney Uptake of 68Ga-Labeled DOTA-Conjugated Peptides and Peptidomimetics. Molecules 2020, 25 (17), 3854.
23. Vaidyanathan, G.; Kang, C. M.; McDougald, D.; Minn, I.; Brummet, M.; Pomper, M. G.; Zalutsky, M. R., Brush border enzyme-cleavable linkers: Evaluation for reducing renal uptake of radiolabeled prostate-specific membrane antigen inhibitors. Nuclear Medicine and Biology 2018, 62-63, 18-30.
24. Radiopharmaceutical Agents (Chinese Patent Application Publication No. CN111065646A published Apr. 24, 2020).
25. Novel radiometal-binding compounds for diagnosis or treatment of prostate specific membrane antigen-expressing cancer (Canadian Patent Application Publication No. CA3079906A1 published Apr. 25, 2019).
26. Trifunctional constructs with tunable pharmacokinetics useful in imaging and anti-tumor therapies (US Patent Application Publication No. 2019/0015531 A1 published Sep. 18, 2018).
27. Folate conjugates of albumin-binding entities (U.S. Pat. No. 9,295,739 B2 filed Aug. 10, 2012).

1. A conjugate of formula I or formula II:
FRTL-BBMecL-AA (formula I)
or
FRTL-Alb-BBMecL-AA (formula II),
wherein:
FRTL is a folate receptor-targeting ligand,
BBMecL is a kidney brush border membrane (BBM) enzyme-cleavable linker,
Alb is an albumin-binder moiety, and
AA is an active agent.

2. The conjugate of claim 1, wherein FRTL has the structure: 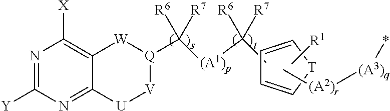
in which:
T is selected from the group consisting of S, O, NR^{4b}, and —HC═CH—,
U, V, and W represent divalent moieties, each independently selected from the group consisting of —(R^{6a})C═, —N═, —(R^{6a})C(R^{7a})—, and —N(R^{4a})—, wherein R^{6a} and R^{7a} are each independently selected from the group consisting of hydrogen, halo, and C₁-C₁₂ alkoxy; or R^{6a} and R^{7a} are taken together to form a carbonyl group,
X and Y are each independently selected from the group consisting of halo, R², OR², SR³, and NR⁴R⁵,
Q is C or CH,
A¹ and A² are each independently selected from the group consisting of oxygen, sulfur, —C(Z)—, —C(Z)O—, —OC(Z)—, —N(R^{4b})—, —C(Z)N(R^{4b})—, —N(R^{4b})C(Z)—, —OC(Z)N(R^{4b})—, —N(R^{4b})C(Z)O—, —N(R^{4b})C(Z)N(R^{5b})—, —S(O)—, —S(O)₂—, —N(R^{4b})S(O)₂—, —C(R^{6b})(R^{7b})—, —N(C≡CH)—, —N(CH₂C≡CH)—, C₁-C₁₂ alkylene, and C₁-C₁₂ alkyeneoxy, where Z is oxygen or sulfur,
R², R³, R⁴, R^{4a}, R^{4b}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl,
A³ is an amino acid,
R¹ is selected from the group consisting of hydrogen, halo, C₁-C₁₂ heteroalkyl, and C₁-C₁₂ alkoxy,
R⁶ and R⁷ are each independently selected from the group consisting of hydrogen, halo, C₁-C₁₂ alkyl, and C₁-C₁₂ alkoxy; or R⁶ and R⁷ are taken together to form a carbonyl group,
q is an integer from 1 to 3,
p, r, s, and t are each independently 0 or 1, and
* indicates the point of attachment to the BBMecL or the Alb.

3. The conjugate of claim 2, wherein Q is CH and/or X is OH and Y is NH₂.

4. (canceled)

5. The conjugate of claim 2, wherein W and U are —N(R^{4a})—; Q is CH; V is CH₂; A¹ is —N(R^{4b})—; s is 1; p is 1; and t is 0.

6. (canceled)

7. The conjugate of claim 2, wherein R^{4a} and R^{4b} are independently alkyl or heteroalkyl.

8. The conjugate of claim 7, wherein R^{4a} and R^{4b} are methyl.

9-14. (canceled)

15. The conjugate of claim 2, wherein FRTL has the structure: 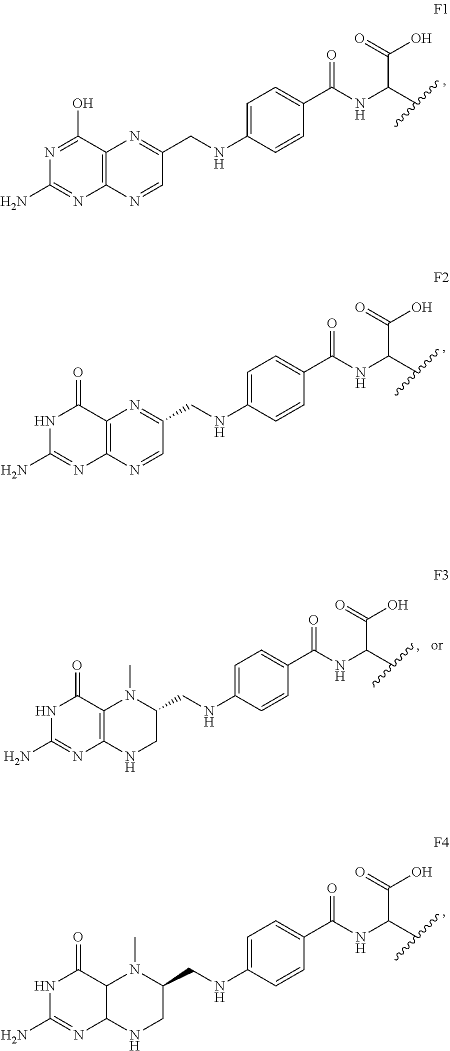
wherein 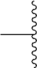
indicates a point of attachment of the FRTL to Alb or BBMecL.

16. The conjugate of claim 1, wherein Alb has the structure: 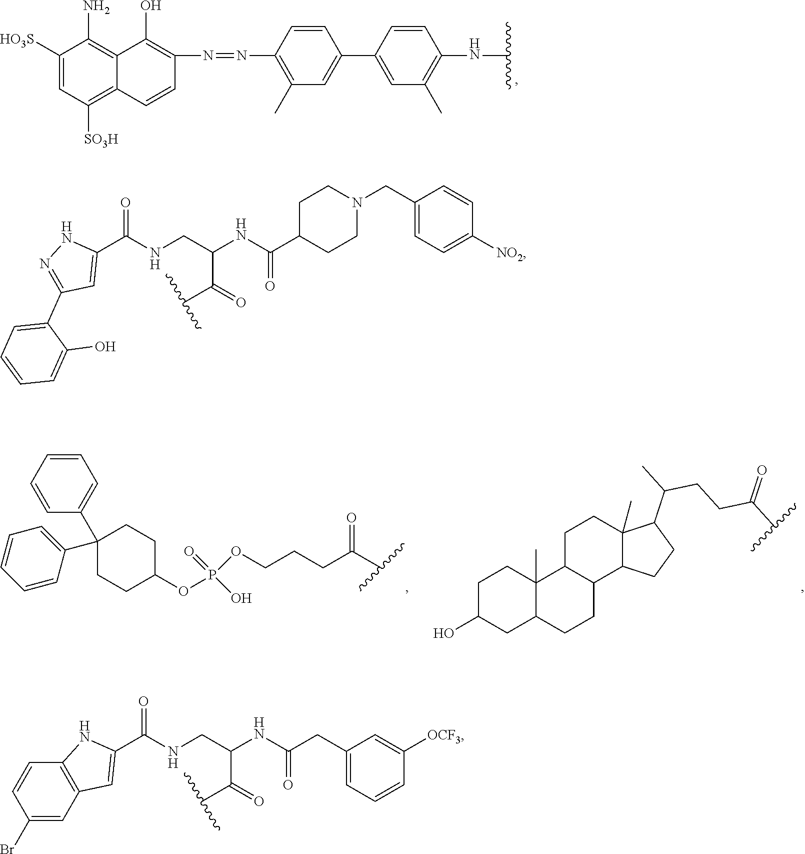 [SMILES: *Nc1ccc(-c2ccc(N=Nc3ccc4c(S(=O)(=O)O)cc(S(=O)(=O)O)c(N)c4c3O)c(C)c2)cc1C ; *C(=O)C(CNC(=O)c1cc(-c2ccccc2O)n[nH]1)NC(=O)C1CCN(Cc2ccc([N+](=O)[O-])cc2)CC1 ; *C(=O)CCCOP(=O)(O)OC1CCC(c2ccccc2)(c2ccccc2)CC1 ; *C(=O)CCC(C)C1CCC2C3CCC4CC(O)CCC4(C)C3CCC12C ; *C(=O)C(CNC(=O)c1cc2cc(Br)ccc2[nH]1)NC(=O)Cc1cccc(OC(F)(F)F)c1] 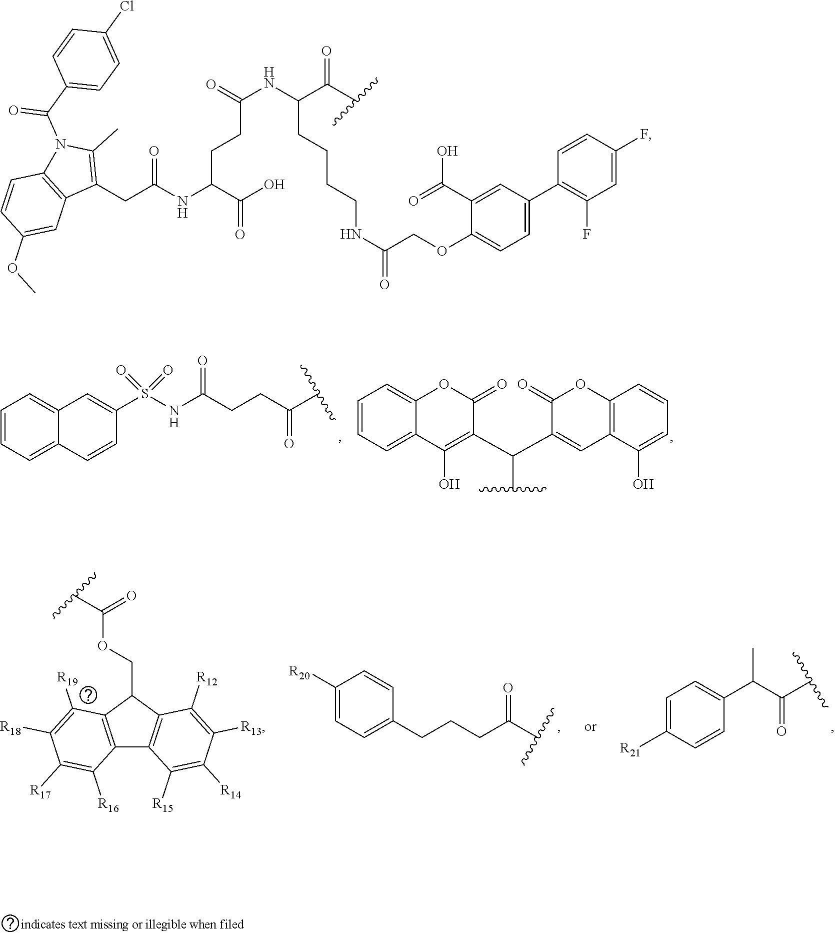
wherein 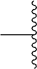
indicates a point of attachment of Alb to FRTL;
R₁₂₋₁₉ are independently —H, —C₁-C₆ alkyl, —F, —Cl, —Br, —I, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, or —SO₂F; and R₂₀ and R₂₁ are independently —H, —C₁-C₆ alkyl, —F, —Cl, —Br, —I, —O—C₁₋₆ alkyl, —CN, —CHO, —B(OH)₂, —C═C—C(O)aryl, —C═C—S(O)₂aryl, —CO₂H, —SO₃H, —SO₂NH₂, —PO₃H₂, —SO₂F, or CF₃, 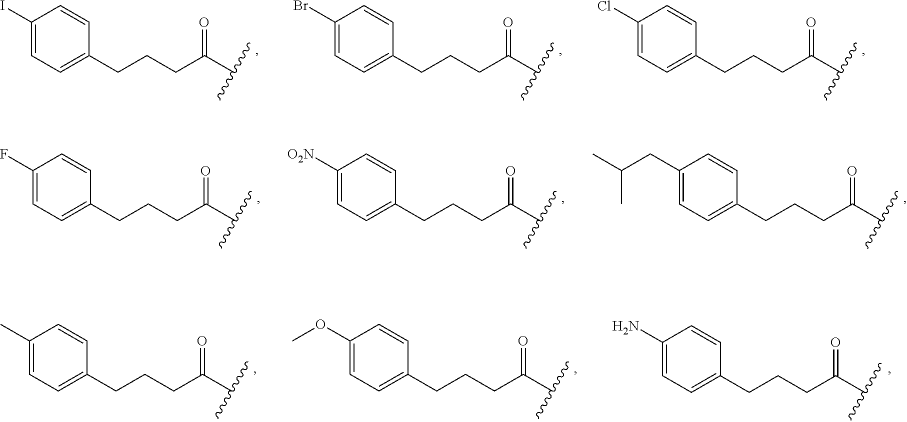 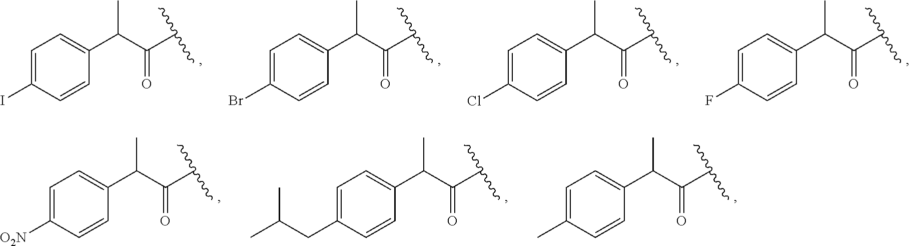 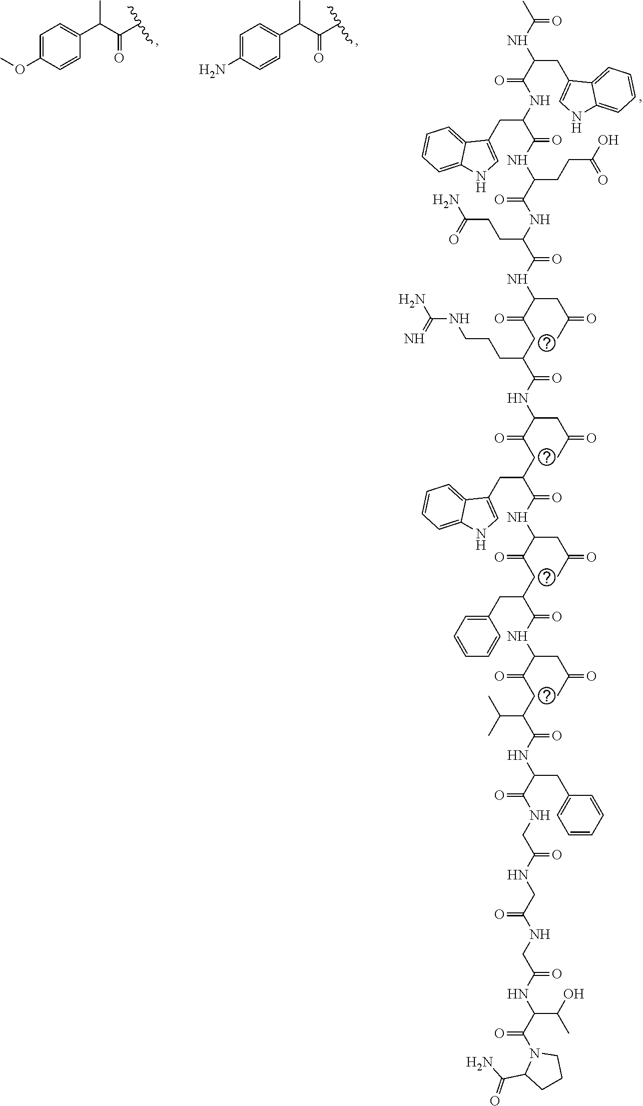 [SMILES: *C(=O)C(C)c1ccc(OC)cc1 ; *C(=O)C(C)c1ccc(N)cc1 ; CC(=O)CC(NC(=O)C(CCCNC(=N)N)CC(=O)C(CC(C)=O)NC(=O)C(CCC(N)=O)NC(=O)C(CCC(=O)O)NC(=O)C(Cc1c[nH]c2ccccc12)NC(=O)C(Cc1c[nH]c2ccccc12)NC(C)=O)C(=O)CC(Cc1c[nH]c2ccccc12)C(=O)NC(CC(C)=O)C(=O)CC(Cc1ccccc1)C(=O)NC(CC(C)=O)C(=O)CC(C(=O)NC(Cc1ccccc1)C(=O)NCC(=O)NCC(=O)NCC(=O)NC(C(=O)N1CCCC1C(N)=O)C(C)O)C(C)C] 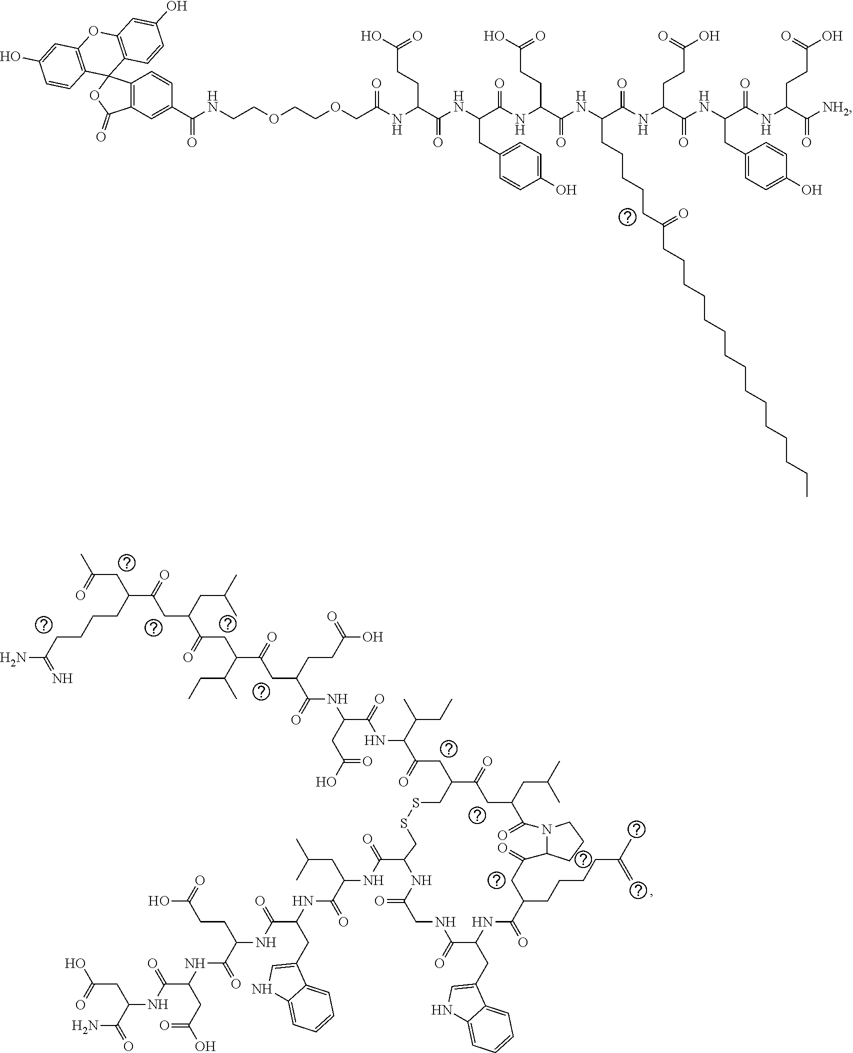 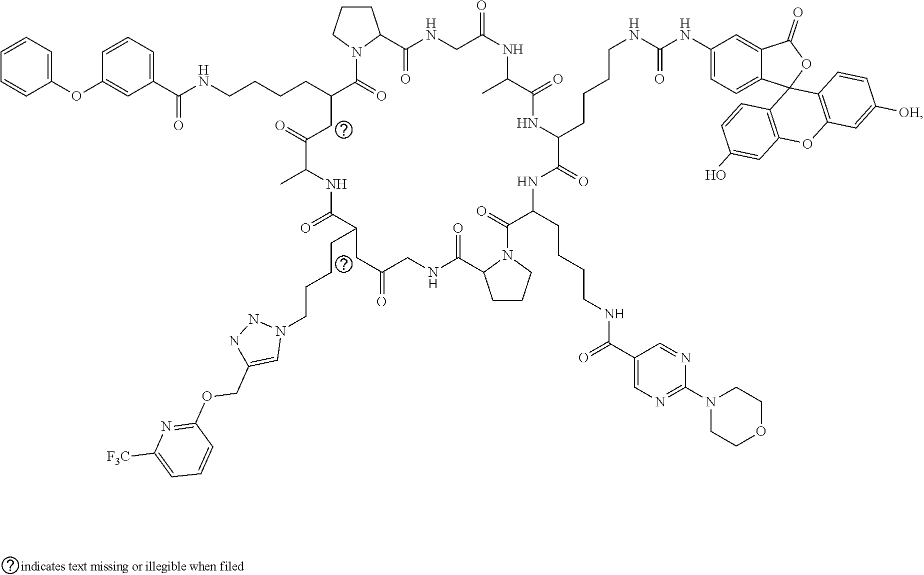 [SMILES: CC1NC(=O)C(CCCCN2C=C(COc3cccc(C(F)(F)F)n3)NN2)CC(=O)CNC(=O)C2CCCN2C(=O)C(CCCCNC(=O)c2cnc(N3CCOCC3)nc2)NC(=O)C(CCCCNC(=O)Nc2ccc3c(c2)C(=O)OC32c3ccc(O)cc3Oc3cc(O)ccc32)NC(=O)C(C)NC(=O)CNC(=O)C2CCCN2C(=O)C(CCCCNC(=O)c2cccc(Oc3ccccc3)c2)CC1=O]
ABD035, ABDCon, designed ankyrin repeat proteins (DARPins), dsFV CA645, a nanobody, or a variable domain of new antigen receptor (VNAR) fused with anti-human serum albumin domain clone E06.

17. The conjugate of claim 2, wherein BBMecL comprises:
(i) Met-Val,
(ii) X-Lys or X-Arg, in which X=Gly or Arg, or a combination thereof,
(iii) Gly-Tyr,
(iv) Gly-Phe-(Lys),
(v) Gly-Pro,
(vi) Ala-X or Leu-X, wherein X is any amino acid, or a combination thereof,
(vii) Asp-X or Glu-X, wherein X is any amino acid, or a combination thereof,
(viii) 7-Glu-X, wherein X is any amino acid, and an acceptor peptide,
(ix) sucrose, maltose, trehalose, lactose, palatinose, or a combination of two or more of the foregoing,
(x) iodoinsulin B chain,
(xi) phlorizin,
(xii) p-nitrophenylphosphate, or
(xiii) a combination of two or more of the foregoing.

18. The conjugate of claim 1, wherein AA is an optical imaging agent, a radioactive imaging agent, or a radioactive therapeutic agent.

19. The conjugate of claim 1, wherein the AA is an optical imaging agent comprising a fluorescent dye selected from the group consisting of S0456, fluorescein isothiocyanate (FITC), rhodamine, LS288, heptamethine cyanine dye (HMCD), SS180, acridine orange (AO), IRDye800CW, IR783, IR825, ZW800-1, and indocyanine green (ICG).

20. (canceled)

21. The conjugate of claim 1, wherein AA is a radioactive imaging agent or radioactive therapeutic agent comprising a radioisotope selected from the group consisting of ¹⁸F, ⁴⁴Sc, ⁴⁷Sc, ⁵²Mn, ⁵⁵Co, ⁶⁴Cu, ⁶⁷Cu, ⁶⁷Ga, ⁶⁸Ga, ⁸⁶Y, ⁸⁹Zr, ⁹⁰Y, ^{99m}Tc, ¹¹¹In, ^{114m}In, ^{117m}Sn, ¹²⁴I, ¹²⁵I, ¹³¹I, ¹⁴⁹Tb, ¹⁵³Sm, ¹⁵²Tb, ¹⁵⁵Tb, ¹⁶¹Tb, ¹⁷⁷Lu, ¹⁸⁶Re, ¹⁸⁸Re, ²¹²Pb, ²¹²Bi, ²¹³Bi, ²²³Ra, ²²⁴Ra, ²²⁵Ab, ²²⁵Ac, and ²²⁷Th.

22. The conjugate of claim 1, wherein AA comprises a radiolabeled prosthetic group comprising a radioisotope selected from the group consisting of ⁶⁸Ga, ¹⁸F, ⁹⁰Y, ^{99m}Tc, ¹¹¹In, ¹⁷⁷Lu, ²²⁵Ac, ¹⁸P, ¹²⁴I, ¹²⁵I, ¹³¹I, and ²¹¹At.

23. The conjugate of claim 22, wherein the radiolabeled prosthetic group comprises a structure selected from: 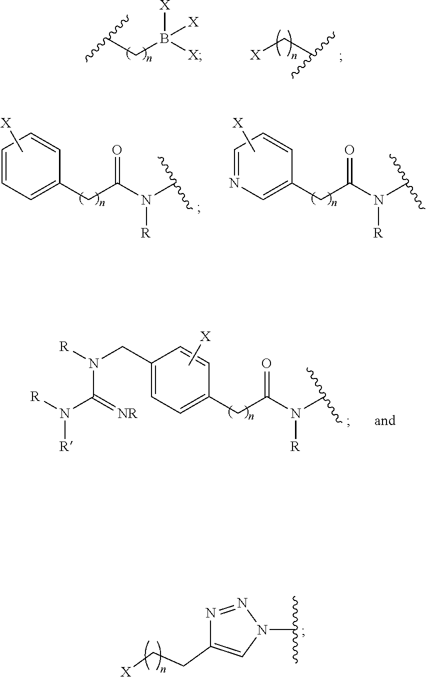
wherein R and R′ are independently hydrogen or methyl, and n is an integer selected from the group consisting of 0, 1, 2, 3, 4, 5, 6, 7, 8, 9, 10, 11, 12, 13, 14, 15, 16, 17, 18, 19 and 20.

24. (canceled)

25. The conjugate of claim 1, wherein AA comprises a chelating agent selected from the group consisting of DOTA (1,4,7,10-tetraazacyclododecane-1,4,7,10-tetraacetic acid), TETA (1,4,8,11-tetraazacyclotetradecane-1,4,8,11-tetraacetic acid), SarAr (1-N-(4-Aminobenzyl)-3,6,10,13,16,19-hexaazabicyclo[6.6.6]-eicosane-1,8-diamine, NOTA (1,4,7-triazacyclononane-1,4,7-triacetic acid), NODAGA (2,2′-(7-(4-((2-aminoethyl)amino)-1-carboxy-4-oxobutyl)-1,4,7-triazonane-1,4-diyl)diacetic acid), HYNIC (6-Hydrazinonicotinic acid), NETA (4-[2-(bis-carboxymethylamino)-ethyl]-7-carboxymethyl-[1,4,7]triazonan-1-yl) acetic acid, TRAP (1,4,7-triazacyclononane-1,4,7-tris[methyl(2-carboxyethyl)phosphinic acid), HBED (N,N-bis(2-hydroxybenzyl)-ethylenediamine-N,N-diacetic acid), 2,3-HOPO (3-hydroxypyridin-2-one), PCTA (3,6,9,15-tetraazabicyclo[9.3.1]-pentadeca-1(15),11,13-triene-3,6,9,-triacetic acid), DFO (desferrioxamine), DTPA (diethylenetriaminepentaacetic acid), OCTAPA (N,N-bis(6-carboxy-2-pyridylmethyl)-ethylenediamine-N,N-diacetic acid), H₂macropa (N,N′-bis[(6-carboxy-2-pyridipmethyl]-4,13-diaza-18-crown-6), H₂dedpa (1,2-[[carboxy)-pyridin-2-yl]-methylamino]ethane, EC20-head comprising β-l-diaminopropionic acid, aspartic acid, and cysteine, and a derivative of any of the foregoing.

26. (canceled)

27. The conjugate of claim 1, which has the structure:

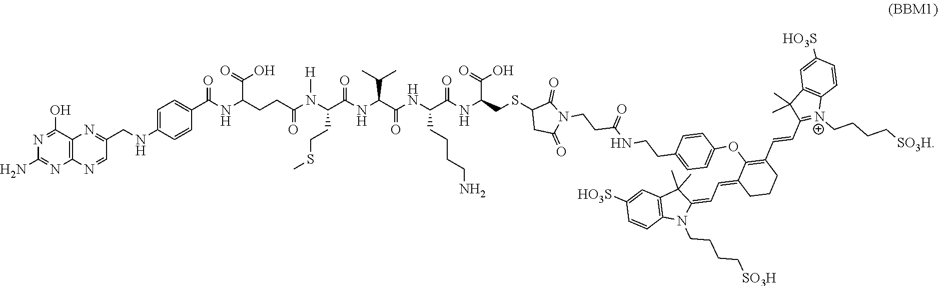 [SMILES: CSCC[C@H](NC(=O)CCC(NC(=O)c1ccc(NCc2cnc3nc(N)nc(O)c3n2)cc1)C(=O)O)C(=O)N[C@H](C(=O)N[C@@H](CCCCN)C(=O)N[C@H](CSC1CC(=O)N(CCC(=O)NCCc2ccc(OC3=C(/C=C/C4=[N+](CCCCS(=O)(=O)O)c5ccc(S(=O)(=O)O)cc5C4(C)C)CCC/C3=C\C=C3\N(CCCCS(=O)(=O)O)c4ccc(S(=O)(=O)O)cc4C3(C)C)cc2)C1=O)C(=O)O)C(C)C]

28. The conjugate of claim 1, which has the structure:

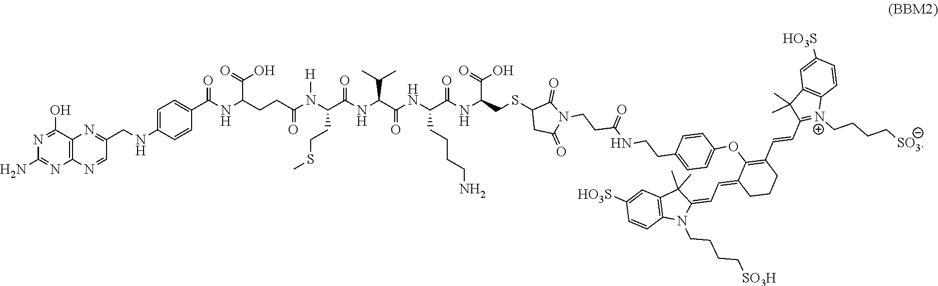

29. The conjugate of claim 1, which has the structure.

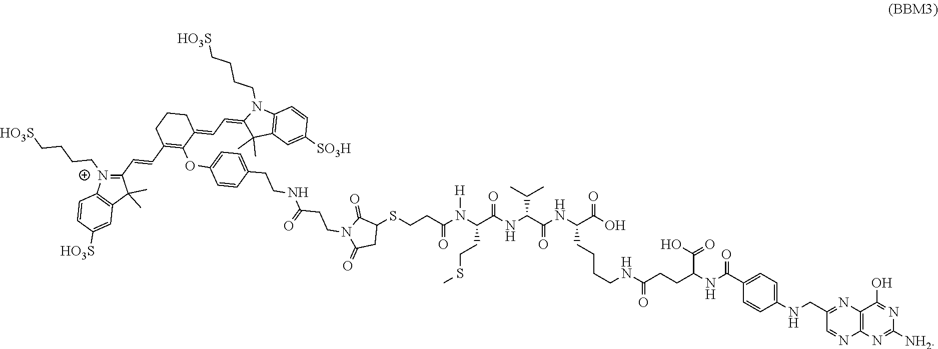 [SMILES: CSCC[C@H](NC(=O)CCSC1CC(=O)N(CCC(=O)NCCc2ccc(OC3=C(/C=C/C4=[N+](CCCCS(=O)(=O)O)c5ccc(S(=O)(=O)O)cc5C4(C)C)CCC/C3=C\C=C3\N(CCCCS(=O)(=O)O)c4ccc(S(=O)(=O)O)cc4C3(C)C)cc2)C1=O)C(=O)N[C@@H](C(=O)N[C@@H](CCCCNC(=O)CCC(NC(=O)c1ccc(NCc2cnc3nc(N)nc(O)c3n2)cc1)C(=O)O)C(=O)O)C(C)C]

30. The conjugate of claim 1, which has the structure:

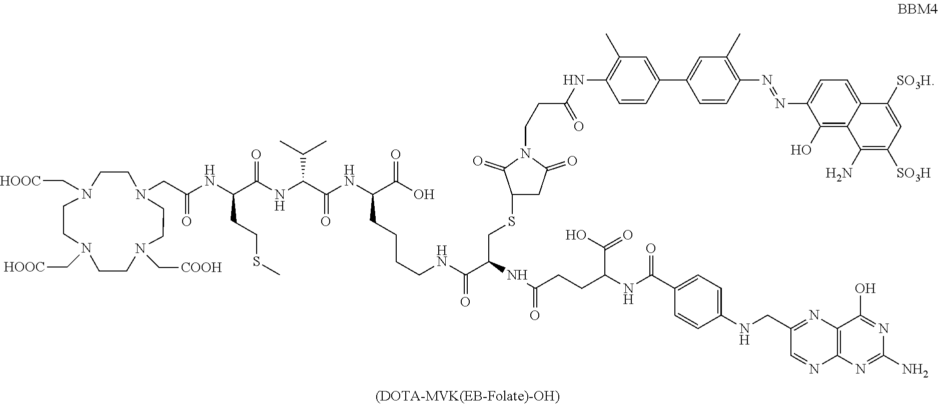 [SMILES: CSCC[C@@H](NC(=O)CN1CCN(CC(=O)O)CCN(CC(=O)O)CCN(CC(=O)O)CC1)C(=O)N[C@@H](C(=O)N[C@H](CCCCNC(=O)[C@@H](CSC1CC(=O)N(CCC(=O)Nc2ccc(-c3ccc(/N=N/c4ccc5c(S(=O)(=O)O)cc(S(=O)(=O)O)c(N)c5c4O)c(C)c3)cc2C)C1=O)NC(=O)CCC(NC(=O)c1ccc(NCc2cnc3nc(N)nc(O)c3n2)cc1)C(=O)O)C(=O)O)C(C)C]

31. The conjugate of claim 1, which has the structure:

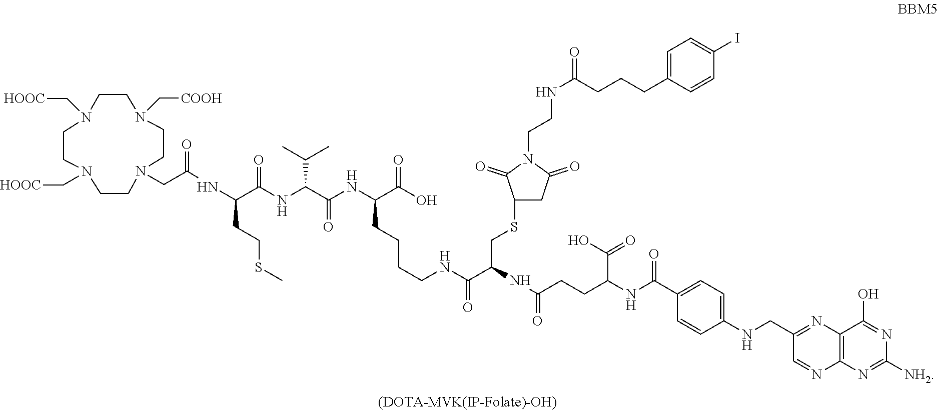 [SMILES: CSCC[C@@H](NC(=O)CN1CCN(CC(=O)O)CCN(CC(=O)O)CCN(CC(=O)O)CC1)C(=O)N[C@@H](C(=O)N[C@H](CCCCNC(=O)[C@@H](CSC1CC(=O)N(CCNC(=O)CCCc2ccc(I)cc2)C1=O)NC(=O)CCC(NC(=O)c1ccc(NCc2cnc3nc(N)nc(O)c3n2)cc1)C(=O)O)C(=O)O)C(C)C]

32. The conjugate of claim 1, which has the structure:

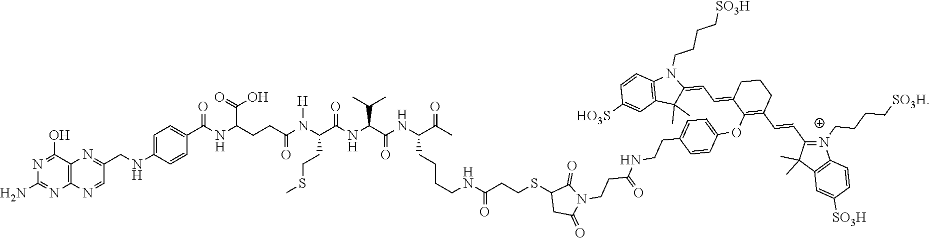 [SMILES: CSCC[C@H](NC(=O)CCC(NC(=O)c1ccc(NCc2cnc3nc(N)nc(O)c3n2)cc1)C(=O)O)C(=O)N[C@H](C(=O)N[C@@H](CCCCNC(=O)CCSC1CC(=O)N(CCC(=O)NCCc2ccc(OC3=C(/C=C/C4=[N+](CCCCS(=O)(=O)O)c5ccc(S(=O)(=O)O)cc5C4(C)C)CCC/C3=C\C=C3\N(CCCCS(=O)(=O)O)c4ccc(S(=O)(=O)O)cc4C3(C)C)cc2)C1=O)C(C)=O)C(C)C]

33. The conjugate of claim 1, which has the structure:

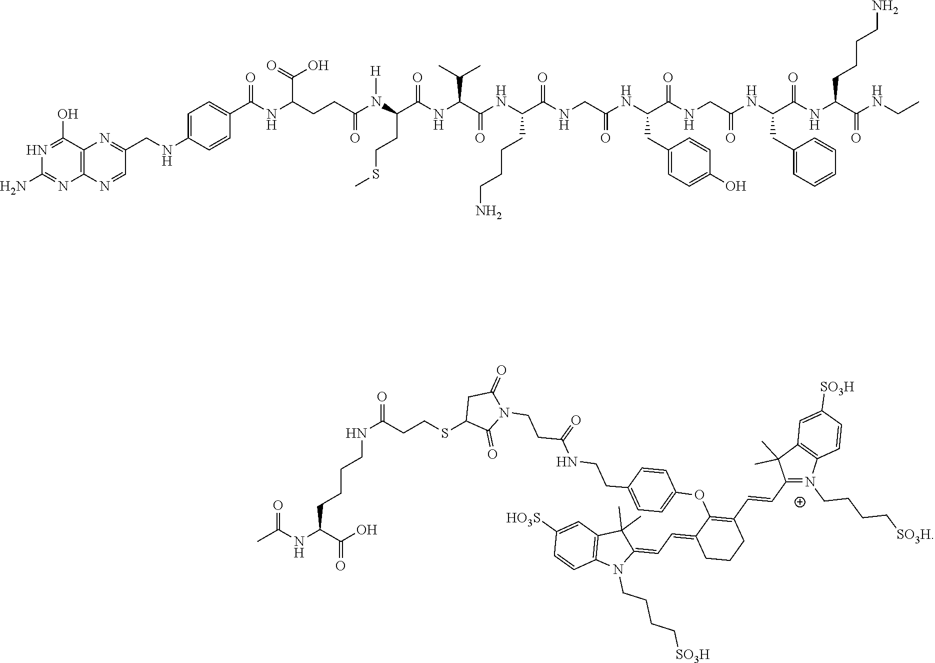

34. The conjugate of claim 1, which has the structure:

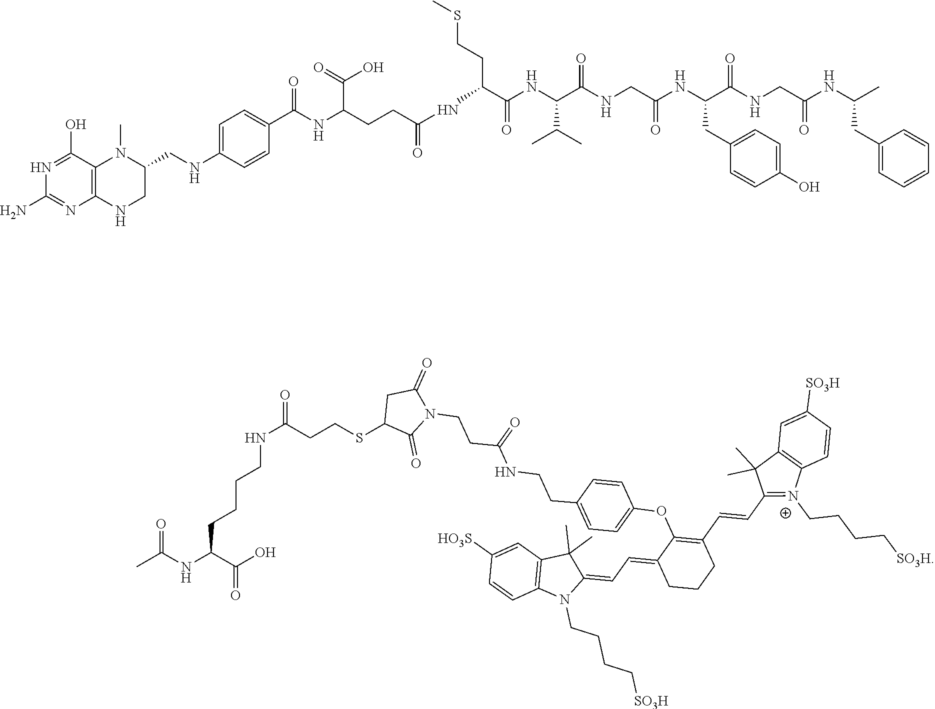 [SMILES: CSCC[C@@H](NC(=O)CCC(NC(=O)c1ccc(NC[C@@H]2CNc3nc(N)nc(O)c3N2C)cc1)C(=O)O)C(=O)N[C@H](C(=O)NCC(=O)N[C@@H](Cc1ccc(O)cc1)C(=O)NCC(=O)N[C@H](C)Cc1ccccc1)C(C)C ; CC(=O)N[C@@H](CCCCNC(=O)CCSC1CC(=O)N(CCC(=O)NCCc2ccc(OC3=C(/C=C/C4=[N+](CCCCS(=O)(=O)O)c5ccc(S(=O)(=O)O)cc5C4(C)C)CCC/C3=C\C=C3\N(CCCCS(=O)(=O)O)c4ccc(S(=O)(=O)O)cc4C3(C)C)cc2)C1=O)C(=O)O]

35. The conjugate of claim 1, which has the structure:

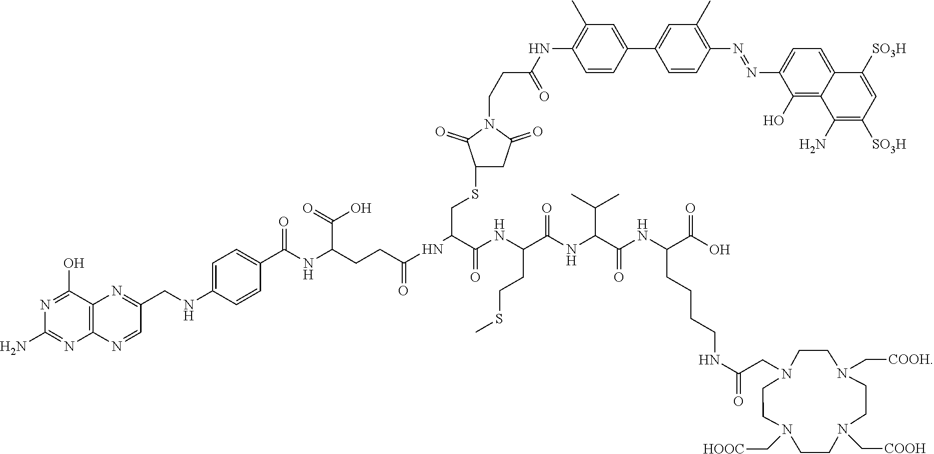 [SMILES: CSCCC(NC(=O)C(CSC1CC(=O)N(CCC(=O)Nc2ccc(-c3ccc(/N=N/c4ccc5c(S(=O)(=O)O)cc(S(=O)(=O)O)c(N)c5c4O)c(C)c3)cc2C)C1=O)NC(=O)CCC(NC(=O)c1ccc(NCc2cnc3nc(N)nc(O)c3n2)cc1)C(=O)O)C(=O)NC(C(=O)NC(CCCCNC(=O)CN1CCN(CC(=O)O)CCN(CC(=O)O)CCN(CC(=O)O)CC1)C(=O)O)C(C)C]

36. The conjugate of claim 1, which has the structure:

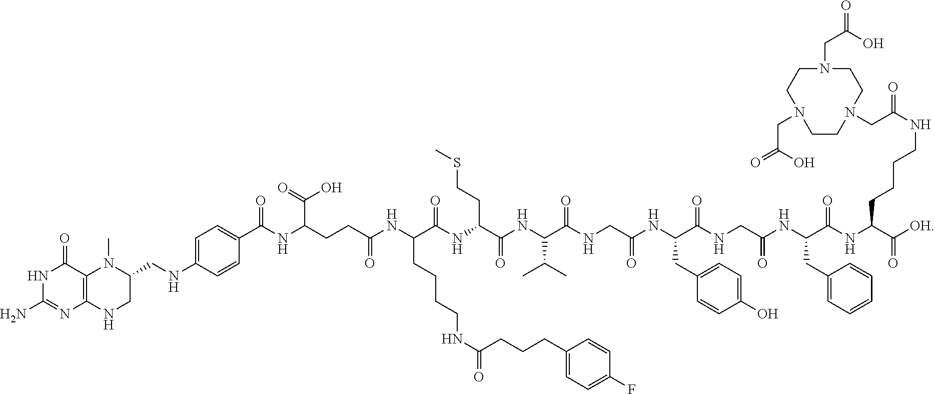

37. The conjugate of claim 1, which has the structure:

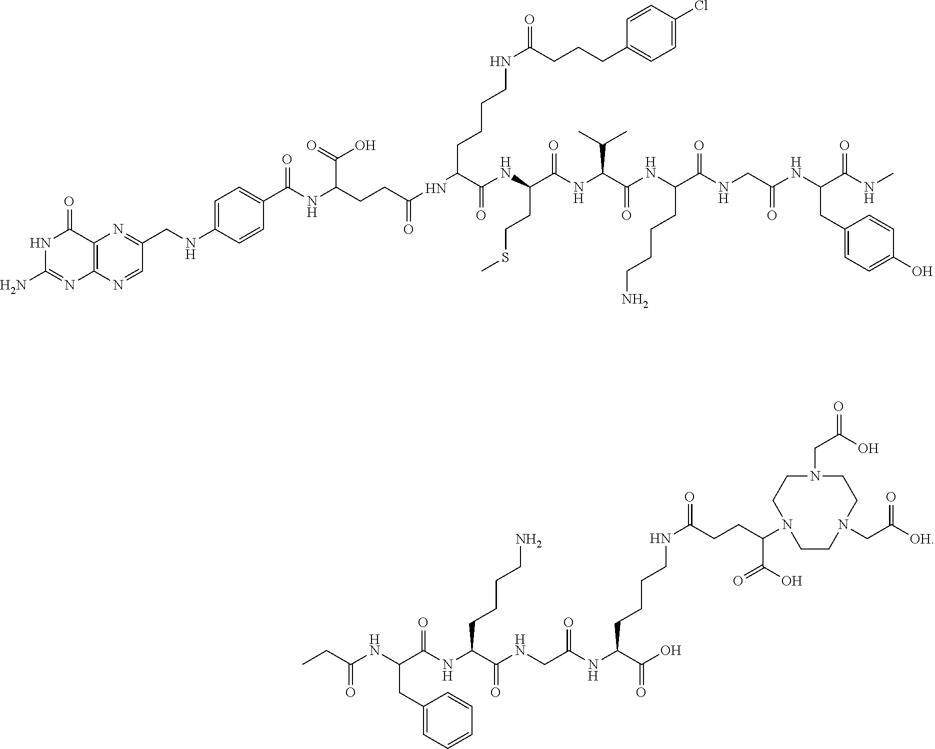 [SMILES: CNC(=O)C(Cc1ccc(O)cc1)NC(=O)CNC(=O)C(CCCCN)NC(=O)[C@@H](NC(=O)[C@@H](CCSC)NC(=O)C(CCCCNC(=O)CCCc1ccc(Cl)cc1)NC(=O)CCC(NC(=O)c1ccc(NCc2cnc3nc(N)[nH]c(=O)c3n2)cc1)C(=O)O)C(C)C ; CCC(=O)NC(Cc1ccccc1)C(=O)N[C@@H](CCCCN)C(=O)NCC(=O)N[C@@H](CCCCNC(=O)CCC(C(=O)O)N1CCN(CC(=O)O)CCN(CC(=O)O)CC1)C(=O)O]

38. A composition comprising a conjugate of claim 1 and a pharmaceutically acceptable carrier.

39. A method of imaging, treating, or imaging and treating a cancer in a subject by targeted radioactivity, alone or in further combination with an optical imaging agent, to cells of a tumor, macrophages associated with the tumor or both, which method comprises administering to the subject an effective amount of a conjugate of claim 1.

40. The method of claim 39, wherein the macrophage associated with the tumor comprise tumor-associated macrophages (TAMs).

41. The method of claim 39, further comprising imaging the tumor.

42-46. (canceled)
